(19) United States
(12) Patent Application Publication
(10) Pub. No.: US 2026/0137789 A1
(43) Pub. Date: May 21, 2026

(54) BRUSH PRODRUGS AND USES THEREOF

(71) Applicant: Massachusetts Institute of Technology, Cambridge, MA (US)

(72) Inventors: Jeremiah A. Johnson, Boston, MA (US); Yivan Jiang, Revere, MA (US); Hung Vanthanh Nguyen, Braintree, MA (US)

(73) Assignee: Massachusetts Institute of Technology, Cambridge, MA (US)

(21) Appl. No.: 19/388,797

(22) Filed: Nov. 13, 2025

Related U.S. Application Data
(63) Continuation of application No. 17/072,015, filed on Oct. 15, 2020, now Pat. No. 12,478,683.
(60) Provisional application No. 62/916,036, filed on Oct. 16, 2019.

(51) Int. Cl. A61K 47/60; A61K 45/06; A61K 47/54; C08F 4/74; C08G 61/08

(52) U.S. Cl. CPC A61K 47/60; A61K 47/545; C08F 4/74; C08G 61/08; A61K 45/06; C08G 2261/1428; C08G 2261/1432; C08G 2261/418; C08G 2261/80; C08G 2261/90

(57) ABSTRACT

The present disclosure provides, in some aspects, macromonomers of Formula (I), and salts thereof; methods of preparing the macromonomers, and salts thereof; Brush prodrugs (polymers); methods of preparing the Brush prodrugs; compounds of Formula (II); conjugates of Formula (III), and salts thereof; pharmaceutical compositions comprising a Brush prodrug, or a conjugate or a salt thereof; kits comprising: a macromonomer or a salt thereof, a Brush prodrug, a compound, a conjugate or a salt thereof, or a pharmaceutical composition; methods of using the Brush prodrugs, or conjugates or salts thereof; and uses of the Brush prodrugs, and conjugates or salts thereof. These chemical entities may be useful in delivering pharmaceutical agents to a subject or cell.

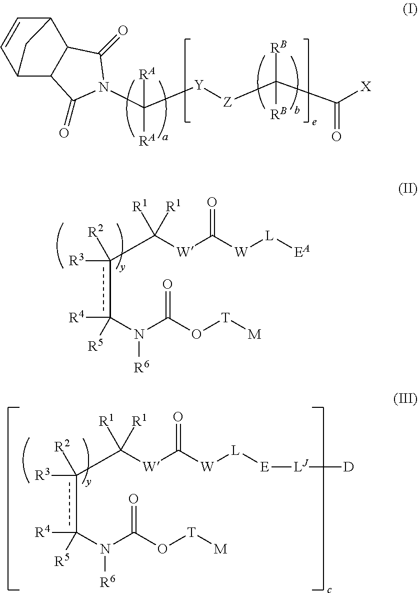

### RELATED APPLICATIONS

The present application claims priority to U.S. Provisional Application No. 62/916,036, filed Oct. 16, 2019, which is incorporated herein by reference.

### BACKGROUND OF THE DISCLOSURE

Bottlebrush polymers have found widespread applications in fields ranging from drug delivery and molecular imaging to novel materials preparation.¹⁻³ Graft-through ring-opening metathesis polymerization (ROMP) offers distinct advantages over other bottlebrush synthesis methods.^{4, 5} The fast-initiating Grubb's 3^{rd} generation catalyst (Ru) has been shown to sustain propagation of polymer chains with exceptionally high tolerance towards a wide range of sterically-hindered multivalent macromonomers (MMs), reaching high degrees of polymerization and low dispersity values, even at low millimolar concentrations.⁶⁻⁷ Furthermore, using Ru, it is possible to control composition, morphology, and size of final macro molecules, preparing remarkable polymeric architectures such as bottlebrushes and stars.⁷⁻¹¹ Due to high packing density of their side-chains, the backbone of bottlebrush polymers is very rigid and adapts extended morphology with minimal side-chain entanglement.⁶ Recently, self-assembly behaviors of bottlebrush block copolymers (BBCPs) have become an active area of research, as these macromolecules readily undergo phase separation and can be used to design materials with novel mechanical properties in bulk.⁶⁻¹² On the other hand, polymeric star nanoarchitectures offer several valuable features such as tunable nanoscale sizes and shapes that mimic globular biomacromolecules, allowing for extended blood circulation and efficient biodistribution and/or tumor accumulation.¹³⁻¹⁵ These properties make star polymers particularly well-suited for biological applications.¹⁰

### SUMMARY OF THE DISCLOSURE

The present disclosure provides, in some aspects, macromonomers, and salts thereof; methods of preparing the macromonomers, and salts thereof; Brush prodrugs (polymers); methods of preparing the Brush prodrugs; pharmaceutical compositions comprising a Brush prodrug; kits comprising: a macromonomer, or a salt thereof, a Brush prodrug, or a pharmaceutical composition; methods of using the Brush prodrugs; and uses of the Brush prodrugs.

The Brush prodrugs may be polymers prepared by polymerizing a macromonomer of Formula (I):

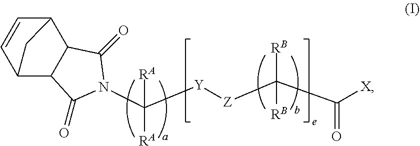

or a salt thereof, in the presence of a metathesis catalyst, wherein each instance of —Y—Z— is independently

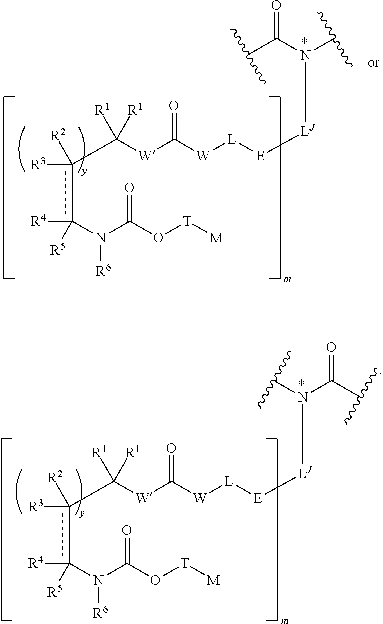

In certain embodiments, the metathesis catalyst is a Grubbs catalyst.

Not bound by any particular theory, the advantages of the Brush prodrugs may be due to the moiety

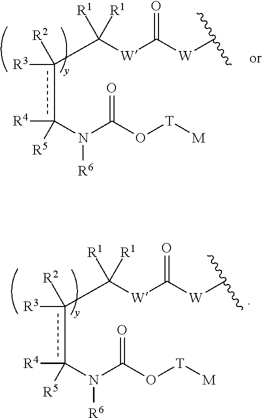

The properties (e.g., release of the free pharmaceutical agents) of the Brush prodrugs may be tuned, e.g., by changing the moiety

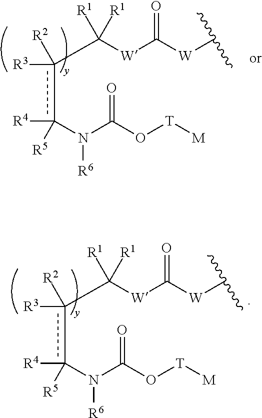

For example, the size, polarity, chemical reactivity, and/or photochemical reactivity of one or more moieties thereof may affect the release (e.g., rate of release) of the pharmaceutical agent. Bulkier moieties may slow the release. Less polar moieties may slow the release. Therefore, the release may be fine tuned by modifying the one or more moieties.

In another aspect, the present disclosure provides compounds of Formula (II):

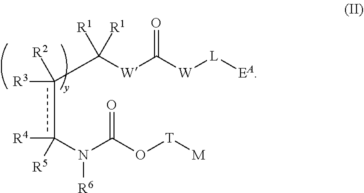

In another aspect, the present disclosure provides conjugates of Formula (III):

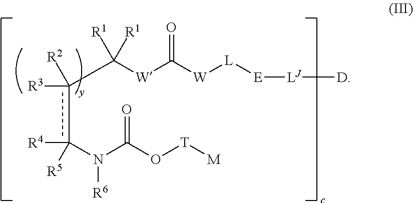

In certain embodiments, each instance of T is substituted or unsubstituted methylene; and each instance of M is independently an ammonium salt or iminium salt of a pharmaceutical agent, wherein the attachment point is the N⁺ of the ammonium salt or iminium salt. The compounds and conjugates may be useful for conjugating with a delivery vehicle a pharmaceutical agent that does not contain a conventional reaction handle.

In another aspect, the present disclosure provides methods of preparing the Brush prodrugs.

In another aspect, the present disclosure provides macromonomers of Formula (I), and salts thereof.

In another aspect, the present disclosure provides methods of preparing the macromonomers, and salts thereof.

In another aspect, the present disclosure provides pharmaceutical compositions comprising a Brush prodrug and optionally a pharmaceutically acceptable excipient.

In another aspect, the present disclosure provides pharmaceutical compositions comprising a conjugate and optionally a pharmaceutically acceptable excipient.

In another aspect, the present disclosure provides kits comprising: a macromonomer, or a salt thereof, a Brush prodrug, or a pharmaceutical composition; and instructions for using the macromonomer, or a salt thereof, the polymer, or the pharmaceutical composition.

In another aspect, the present disclosure provides kits comprising a compound; and instructions for using the compound.

In another aspect, the present disclosure provides kits comprising a conjugate, or a salt thereof, or a pharmaceutical composition; and instructions for using the conjugate, or a salt thereof, or the pharmaceutical composition.

In another aspect, the present disclosure provides methods of delivering a pharmaceutical agent to a subject in need thereof comprising administering to the subject in need thereof a polymer or a pharmaceutical composition.

In another aspect, the present disclosure provides methods of delivering a pharmaceutical agent to a cell comprising contacting the cell with a polymer or a pharmaceutical composition.

In another aspect, the present disclosure provides methods of treating a disease in a subject in need thereof comprising administering to or implanting in the subject in need thereof a therapeutically effective amount of: a polymer or a pharmaceutical composition; wherein at least one instance of the pharmaceutical agent is a therapeutic agent.

In another aspect, the present disclosure provides methods of preventing a disease in a subject in need thereof comprising administering to or implanting in the subject in need thereof a prophylactically effective amount of: a polymer or a pharmaceutical composition; wherein at least one instance of the pharmaceutical agent is a prophylactic agent.

In another aspect, the present disclosure provides methods of diagnosing a disease in a subject comprising administering to or implanting in the subject a diagnostically effective amount of: a polymer or a pharmaceutical composition; wherein at least one instance of the pharmaceutical agent is a diagnostic agent.

In another aspect, the present disclosure provides methods of delivering a pharmaceutical agent to a subject in need thereof comprising administering to the subject in need thereof a conjugate or a pharmaceutical composition.

In another aspect, the present disclosure provides methods of delivering a pharmaceutical agent to a cell comprising contacting the cell with a conjugate or a pharmaceutical composition.

In another aspect, the present disclosure provides methods of treating a disease in a subject in need thereof comprising administering to or implanting in the subject in need thereof a therapeutically effective amount of: a conjugate or a pharmaceutical composition; wherein at least one instance of the pharmaceutical agent is a therapeutic agent.

In another aspect, the present disclosure provides methods of preventing a disease in a subject in need thereof comprising administering to or implanting in the subject in need thereof a prophylactically effective amount of: a conjugate or a pharmaceutical composition; wherein at least one instance of the pharmaceutical agent is a prophylactic agent.

In another aspect, the present disclosure provides methods of diagnosing a disease in a subject comprising administering to or implanting in the subject a diagnostically effective amount of: a conjugate or a pharmaceutical composition; wherein at least one instance of the pharmaceutical agent is a diagnostic agent.

In certain embodiments, the disease is cancer.

The present disclosure refers to various issued patent, published patent applications, journal articles, and other publications, all of which are incorporated herein by reference.

### Definitions

For convenience, certain terms employed herein, in the specification, examples and appended claims are collected herein.

Unless otherwise required by context, singular terms shall include pluralities and plural terms shall include the singular.

The following definitions are more general terms used throughout the present application:

The singular terms “a,” “an,” and “the” include plural referents unless context clearly indicates otherwise. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, and more typically, within 5%, 4%, 3%, 2% or 1% of a given value or range of values.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers. Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

When a range of values (“range”) is listed, it is intended to encompass each value and sub-range within the range. A range is inclusive of the values at the two ends of the range unless otherwise provided. For example, “an integer between 1 and 4” refers to 1, 2, 3, and 4. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” refers to a radical of a C₁-C₁₀₀₀ straight-chain or branched saturated hydrocarbon group. In some embodiments, an alkyl group has 1 to 200 carbon atoms (“C₁-C₂₀₀ alkyl”), 1 to 20 carbon atoms (“C₁-C₂₀ alkyl”), 1 to 10 carbon atoms (“C₁-C₁₀ alkyl”), 1 to 9 carbon atoms (“C₁-C₉ alkyl”), 1 to 8 carbon atoms (“C₁-C₈ alkyl”), 1 to 7 carbon atoms (“C₁-C₇ alkyl”), 1 to 6 carbon atoms (“C₁-C₆ alkyl”), 1 to 5 carbon atoms (“C₁-C₅ alkyl”), 1 to 4 carbon atoms (“C₁-C₄ alkyl”), 1 to 3 carbon atoms (“C₁-C₃ alkyl”), 1 to 2 carbon atoms (“C₁-C₂ alkyl”), or 1 carbon atom (“C₁ alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. C₃₀-C₁₀₀₀ alkyl may be obtained from polymerization. Unless otherwise specified, each instance of an alkyl group is independently unsubstituted (an “unsubstituted alkyl”) or substituted (a “substituted alkyl”) with one or more substituents.

The term “alkenyl” refers to a radical of a straight-chain or branched hydrocarbon group having from 2 to 1000 carbon atoms and one or more carbon-carbon double bonds (e.g., 1, 2, 3, or 4 double bonds). In some embodiments, an alkenyl group has 2 to 200 carbon atoms (“C₂₋₂₀₀ alkenyl”). In some embodiments, an alkenyl group has 2 to 20 carbon atoms (“C₂₋₂₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. C₃₀-C₁₀₀₀ alkenyl may be obtained from polymerization. Unless otherwise specified, each instance of an alkenyl group is independently unsubstituted (an “unsubstituted alkenyl”) or substituted (a “substituted alkenyl”) with one or more substituents. In an alkenyl group, a C═C double bond for which the stereochemistry is not specified (e.g., —CH═CHCH₃,

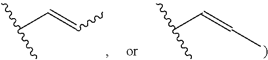

may be in the (E)- or (Z)-configuration.

The term “alkynyl” refers to a radical of a straight-chain or branched hydrocarbon group having from 2 to 1000 carbon atoms and one or more carbon-carbon triple bonds (e.g., 1, 2, 3, or 4 triple bonds) (“C₂₋₁₀ alkynyl”). In some embodiments, an alkynyl group has 2 to 200 carbon atoms (“C₂₋₂₀₀ alkynyl”), 2 to 20 carbon atoms (“C₂₋₂₀ alkynyl”), 2 to 9 carbon atoms (“C₂₋₉ alkynyl”), 2 to 8 carbon atoms (“C₂₋₈ alkynyl”), 2 to 7 carbon atoms (“C₂₋₇ alkynyl”), 2 to 6 carbon atoms (“C₂₋₆ alkynyl”), 2 to 5 carbon atoms (“C₂₋₅ alkynyl”), 2 to 4 carbon atoms (“C₂₋₄ alkynyl”), 2 to 3 carbon atoms (“C₂₋₃ alkynyl”), or 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. C₃₀-C₁₀₀₀ alkynyl may be obtained from polymerization. Unless otherwise specified, each instance of an alkynyl group is independently unsubstituted (an “unsubstituted alkynyl”) or substituted (a “substituted alkynyl”) with one or more substituents.

The term “heteroalkyl” refers to an alkyl group which further includes at least one heteroatom (e.g., 1, 2, 3, 4, or more heteroatoms, as valency permits) selected from oxygen, nitrogen, phosphorus, or sulfur within (i.e., inserted between adjacent carbon atoms of) and/or placed at one or more terminal position(s) of the parent chain. In certain embodiments, a heteroalkyl group refers to a saturated group having from 1 to 1000 carbon atoms and 1 or more heteroatoms within the parent chain (“C₁-C₁₀₀₀ heteroalkyl”), 1 to 20 carbon atoms and 1 or more heteroatoms within the parent chain (“C₁-C₂₀ heteroalkyl”), 1 to 10 carbon atoms and 1 or more heteroatoms within the parent chain (“C₁-C₁₀ heteroalkyl”), 1 to 9 carbon atoms and 1 or more heteroatoms within the parent chain (“C₁-C₉ heteroalkyl”), 1 to 8 carbon atoms and 1 or more heteroatoms within the parent chain (“C₁-C₈ heteroalkyl”), 1 to 7 carbon atoms and 1 or more heteroatoms within the parent chain (“C₁-C₇ heteroalkyl”), 1 to 6 carbon atoms and 1 or more heteroatoms within the parent chain (“C₁-C₆ heteroalkyl”), 1 to 5 carbon atoms and 1 or more heteroatoms within the parent chain (“C₁-C₅ heteroalkyl”), 1 to 4 carbon atoms and for more heteroatoms within the parent chain (“C₁-C₄ heteroalkyl”), 1 to 3 carbon atoms and 1 or more heteroatoms within the parent chain (“C₁-C₃ heteroalkyl”), 1 to 2 carbon atoms and 1 heteroatom within the parent chain (“C₁-C₂ heteroalkyl”), or 1 carbon atom and 1 heteroatom (“C₁ heteroalkyl”). C₃₀-C₁₀₀₀ heteroalkyl may be obtained from polymerization. Unless otherwise specified, each instance of a heteroalkyl group is independently unsubstituted (an “unsubstituted heteroalkyl”) or substituted (a “substituted heteroalkyl”) with one or more substituents.

The term “heteroalkenyl” refers to an alkenyl group, which further includes at least one heteroatom (e.g., 1, 2, 3, 4, or more heteroatoms, as valency permits) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 1000 carbon atoms, at least one double bond, and 1 or more heteroatoms within the parent chain (“heteroC₂₋₁₀₀₀ alkenyl”). In certain embodiments, a heteroalkenyl group refers to a group having from 2 to 20 carbon atoms, at least one double bond, and 1 or more heteroatoms within the parent chain (“heteroC₂₋₂₀ alkenyl”). In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). C₃₀-C₁₀₀₀ heteroalkenyl may be obtained from polymerization. Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “heteroalkynyl” refers to an alkynyl group, which further includes at least one heteroatom (e.g., 1, 2, 3, 4, or more heteroatoms, as valency permits) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 1000 carbon atoms, at least one triple bond, and 1 or more heteroatoms within the parent chain (“heteroC₂₋₁₀₀₀ alkynyl”). In certain embodiments, a heteroalkynyl group refers to a group having from 2 to 20 carbon atoms, at least one triple bond, and 1 or more heteroatoms within the parent chain (“heteroC₂₋₂₀ alkynyl”). In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆alkynyl”). C₃₀-C₁₀₀₀ heteroalkynyl may be obtained from polymerization. Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” or “cycloalkyl” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”), 3 to 7 ring carbon atoms (“C₃₋₇ carbocyclyl”), 3 to 6 ring carbon atoms (“C₃₋₆ carbocyclyl”), 4 to 6 ring carbon atoms (“C₄ carbocyclyl”), 5 to 6 ring carbon atoms (“C₅₋₆ carbocyclyl”), or 5 to 10 ring carbon atoms (“C₅₋₁₀ carbocyclyl”). Exemplary C₃₋₁₀ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, phosphorus, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents.

In some embodiments, a heterocyclyl group is a 5-10 membered non-aromatic ring system having ring carbon atoms and 1-4 ring heteroatoms, wherein each heteroatom is independently selected from nitrogen, oxygen, phosphorus, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, phosphorus, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, phosphorus, and sulfur (“5-6 membered heterocyclyl”). In some embodiments, the 5-6 membered heterocyclyl has 1-3 ring heteroatoms selected from nitrogen, oxygen, phosphorus, and sulfur. In some embodiments, the 5-6 membered heterocyclyl has 1-2 ring heteroatoms selected from nitrogen, oxygen, phosphorus, and sulfur. In some embodiments, the 5-6 membered heterocyclyl has 1 ring heteroatom selected from nitrogen, oxygen, phosphorus, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl, and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 n electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₋₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents.

The term “heteroaryl” refers to a radical of a 5-14 membered monocyclic or polycyclic (e.g., bicyclic, tricyclic) 4n+2 aromatic ring system (e.g., having 6, 10, or 14 n electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group be monovalent or may have more than one point of attachment to another moiety (e.g., it may be divalent, trivalent, etc), although the valency may be specified directly in the name of the group. For example, “triazoldiyl” refers to a divalent triazolyl moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

As understood from the above, alkyl, alkenyl, alkynyl, carbocyclyl, aryl, and heteroaryl groups are, in certain embodiments, optionally substituted. Optionally substituted refers to a group which may be substituted or unsubstituted (e.g., “substituted” or “unsubstituted” alkyl). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Affixing the suffix “ene” to a group indicates the group is a polyvalent (e.g., bivalent, trivalent, tetravalent, or pentavalent) moiety. In certain embodiments, affixing the suffix “ene” to a group indicates the group is a bivalent moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{aa})₂, —OP(═O)(R^{a})₂, —OP(═O)(OR^{bb})₂, —P(═O)(N(R^{bb})₂, —OP(═O)(N(R^{bb})₂, —NR^{bb}P(═O)(R^{aa})₂, —NR^{bb}P(═O)(OR^{cc})₂, —NR^{bb}P(═O)(N(R^{bb})₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{cc})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂. ═NNR^{bb}C(═O)R^{aa}, ═NNR^{bb}C(═O)OR^{aa}, ═NNR^{bb}S(═O)₂R^{aa}, ═NR^{aa}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{ccc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂, C₁₋₁₀ alkyl, C₁₋₁₀ perhaloalkyl, C₂₋₁₀ alkenyl, C₂₋₁₀ alkynyl, heteroC₂₋₁₀alkyl, heteroC₂₋₁₀alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₄₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 RU groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{ff} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl). —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂₀C₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆-10 aryl, 3-10 membered heterocyclyl, 5-10 membered heteroaryl; or two geminal R^{gg} substituents can be joined to form ═O or ═S; wherein X⁻ is a counterion.

In certain embodiments, the carbon atom substituents are independently halogen, substituted or unsubstituted, C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, the carbon atom substituents are independently halogen, substituted or unsubstituted, C₁₋₆ alkyl, —OR^{aa}, —SR^{aa}, —N(R^{bb})₂, —CN, —SCN, or —NO₂.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{aa}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), p-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{cc})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂. —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH.

The term “thiol” or “thio” refers to the group —SH.

The term “amine” or “amino” refers to the group —NH— or —NH₂.

As used herein, the term “polyethylene glycol” or “PEG” refers to an ethylene glycol polymer that contains about 20 to about 2,000,000 linked monomers, typically about 50-1,000 linked monomers, usually about 100-300. Polyethylene glycols include ethylene glycol polymer containing various numbers of linked monomers, e.g., PEG20, PEG30, PEG40, PEG60, PEG80, PEG100, PEG115, PEG200, PEG300, PEG400, PEG500, PEG600, PEG1000, PEG1500, PEG2000, PEG3350, PEG4000, PEG4600, PEG5000, PEG6000, PEG8000, PEG11000, PEG12000, PEG2000000 and any mixtures thereof.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate (triflate), p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes. In certain embodiments, the counterion is triflate.

The term “salt” refers to ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids such as hydrochloric acid, hydrobromic acid, phosphoric acid, sulfuric acid, and perchloric acid, or with organic acids such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “leaving group” is given its ordinary meaning in the art of synthetic organic chemistry and refers to an atom or a group capable of being displaced by a nucleophile. Examples of suitable leaving groups include halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some cases, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some cases, the leaving group is a brosylate, such as p-bromobenzenesulfonyloxy. In some cases, the leaving group is a nosylate, such as 2-nitrobenzenesulfonyloxy. In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate. Other examples of leaving groups are water, ammonia, alcohols, ether moieties, thioether moieties, zinc halides, magnesium moieties, diazonium salts, and copper moieties.

“Click chemistry” reaction includes Huisgen alkyne-azide cycloaddition. Any “click chemistry” reaction known in the art can be used to this end. Click chemistry is a chemical approach introduced by Sharpless in 2001 and describes chemistry tailored to generate substances quickly and reliably by joining small units together. See, e.g., Kolb, Finn and Sharpless Angewandte Chemie International Edition (2001) 40: 2004-2021; Evans, Australian Journal of Chemistry (2007) 60: 384-395). Exemplary coupling reactions (some of which may be classified as “click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition; thiol-yne addition; imine formation; Michael additions (e.g., maleimide addition); and Diels-Alder reactions (e.g., tetrazine [4+2]cycloaddition).

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay and/or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population of subjects.

The terms “condition.” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactically effective amount. In certain embodiments, an effective amount is the amount of a compound or pharmaceutical composition described herein in a single dose. In certain embodiments, an effective amount is the combined amounts of a compound or pharmaceutical composition described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

The term “therapeutic agent” includes an agent that is capable of providing a local or systemic biological, physiological, or therapeutic effect in the biological system to which it is applied. For example, a therapeutic agent can act to control tumor growth, control infection or inflammation, act as an analgesic, promote anti-cell attachment, and enhance bone growth, among other functions. Other suitable therapeutic agents can include anti-viral agents, hormones, antibodies, or therapeutic proteins. Other therapeutic agents include prodrugs, which are agents that are not biologically active when administered but, upon administration to a subject are converted to biologically active agents through metabolism or some other mechanism.

The term “prodrug” refer to a compound that becomes active, e.g., by solvolysis, reduction, oxidation, or under physiological conditions, to provide a pharmaceutically active compound, e.g., in vivo. A prodrug can include a derivative of a pharmaceutically active compound, such as, for example, to form an ester by reaction of the acid, or acid anhydride, or mixed anhydrides moieties of the prodrug moiety with the hydroxyl moiety of the pharmaceutical active compound.

The term “small molecule” refers to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than 2,000 g/mol. In certain embodiments, the molecular weight of a small molecule is not more than 1,500 g/mol. In certain embodiments, the molecular weight of a small molecule is not more than 1,000 g/mol, not more than 900 g/mol, not more than 800 g/mol, not more than 700 g/mol, not more than 600 g/mol, not more than 500 g/mol, not more than 400 g/mol, not more than 300 g/mol, not more than 200 g/mol, or not more than 100 g/mol. In certain embodiments, the molecular weight of a small molecule is at least 100 g/mol, at least 200 g/mol, at least 300 g/mol, at least 400 g/mol, at least 500 g/mol, at least 600 g/mol, at least 700 g/mol, at least 800 g/mol, or at least 900 g/mol, or at least 1,000 g/mol. Combinations of the above ranges (e.g., at least 200 g/mol and not more than 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

A “protein,” “peptide,” or “polypeptide” comprises a polymer of amino acid residues linked together by peptide bonds. The term refers to proteins, polypeptides, and peptides of any size, structure, or function. A protein may refer to an individual protein or a collection of proteins. Proteins preferably contain only natural amino acids, although non-natural amino acids (i.e., compounds that do not occur in nature but that can be incorporated into a polypeptide chain) and/or amino acid analogs as are known in the art may alternatively be employed. In certain embodiments, the amino acid residues of a peptide are alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and/or valine, in D and/or L form. In certain embodiments, the amino acid residues of a peptide are alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and/or valine, in L form. One or more of the amino acids in a protein may be protected. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these. In certain embodiments, a protein comprises between 2 and 10, between 10 and 30, between 30 and 100, between 100 and 300, or between 300 and 1,000, inclusive, amino acids. In certain embodiments, a protein comprises between 1,000 and 3,000, or between 3,000 and 10,000, inclusive, amino acids. In certain embodiments, the amino acids in a protein are natural amino acids. In certain embodiments, the amino acids in a protein are unnatural amino acids. In certain embodiments, the amino acids in a protein are a combination of natural amino acids and unnatural amino acids.

The disclosure is not intended to be limited in any manner by the above exemplary listing of substituents. Additional terms may be defined in other sections of this disclosure.

### BRIEF DESCRIPTION OF THE DRAWINGS

The figures are exemplary and do not limit the scope of the present disclosure.

FIG. 1 shows a ¹H NMR spectrum of JQ-MM in CDCl₃.

FIG. 2 shows a matrix-assisted laser desorption/ionization time-of-flight mass spectrometry (MALDI-TOF MS) spectrum of JQ-MM.

FIG. 3 shows a ¹H NMR spectrum of AZ-MM in CDCl₃.

FIG. 4 shows a MALDI-TOF spectrum of AZ-MM.

FIG. 5 shows a ¹H NMR spectrum of Vin-MM in CDCl₃.

FIG. 6 shows a MALDI-TOF spectrum of Vin-MM.

FIG. 7 shows GPC traces of JQ-BBP, AZ-BBP, and Vin-BBP. Small peak at 15.5 min elution time corresponds to residual MMs. In all cases, reaction conversions were ≥90%.

FIG. 8 shows the hydrodynamic diameter (Dₕ) of JQ-BBP as determined by dynamic light scattering (DLS).

FIG. 9 shows the Dₕ of AZ-BBP as determined by dynamic light scattering DLS.

FIG. 10 shows the Dₕ of Vin-BBP as determined by dynamic light scattering DLS.

FIG. 11 shows a ¹H NMR spectrum of JQ-MM-2 in CDCl₃.

FIG. 12 shows a MALDI-TOF spectrum of JQ-MM-2.

FIG. 13 shows a ¹H NMR spectrum of JQ-MM-3 in CDCl₃.

FIG. 14 shows a MALDI-TOF spectrum of JQ-MM-3.

FIG. 15 shows GPC traces of JQ-BBP, JQ-BBP-2, and JQ-BBP-3. In all cases, reaction conversions were ≥90%.

FIG. 16 shows the Dₕ of JQ-BBP-2 as determined by dynamic light scattering DLS.

FIG. 17 shows the Dₕ of JQ-BBP-3 as determined by dynamic light scattering DLS.

FIG. 18 shows exemplary mechanisms of the release of pharmaceutical agents from exemplary Brush prodrugs described herein.

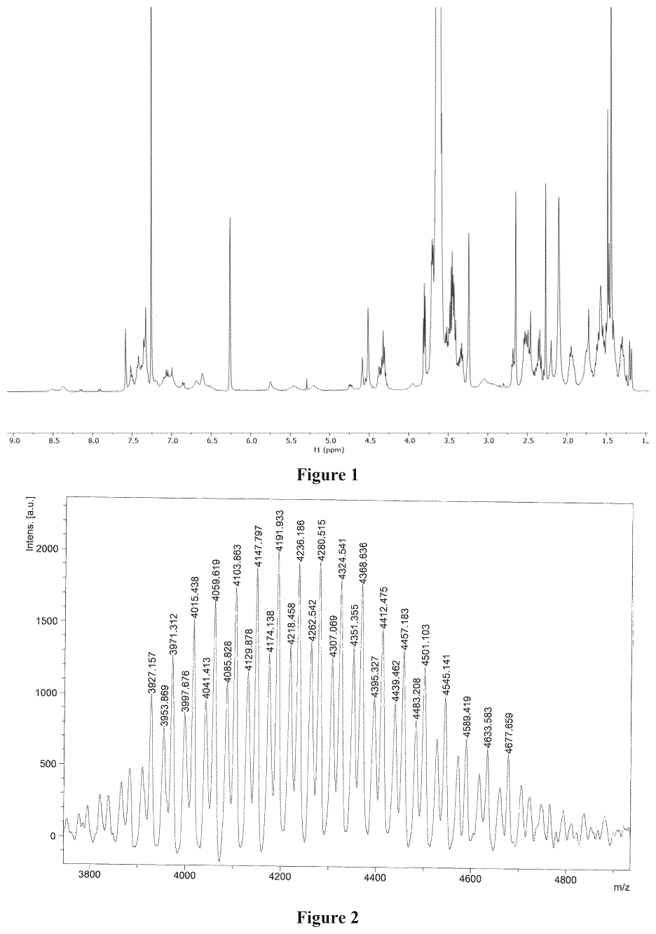

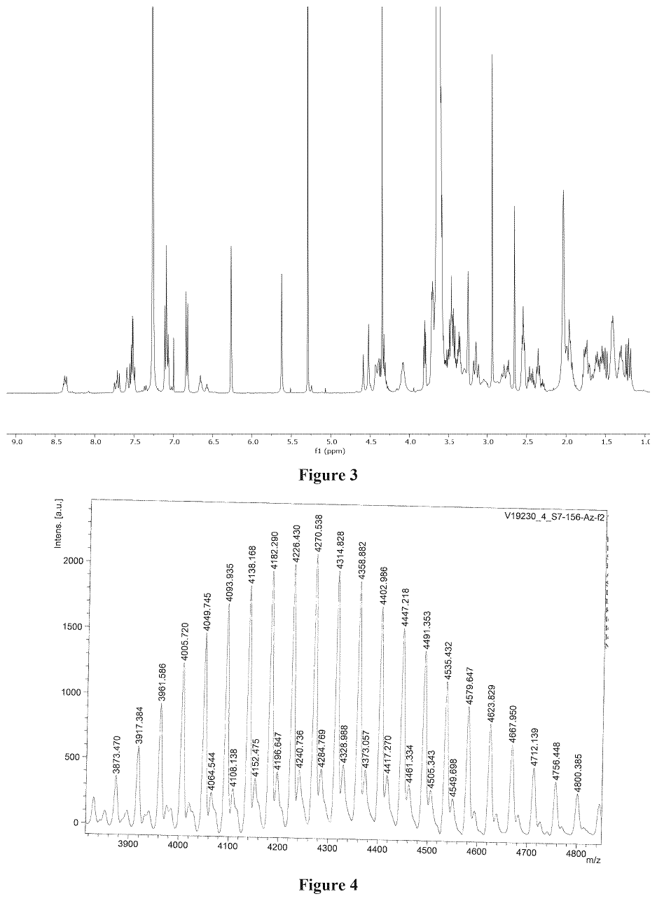

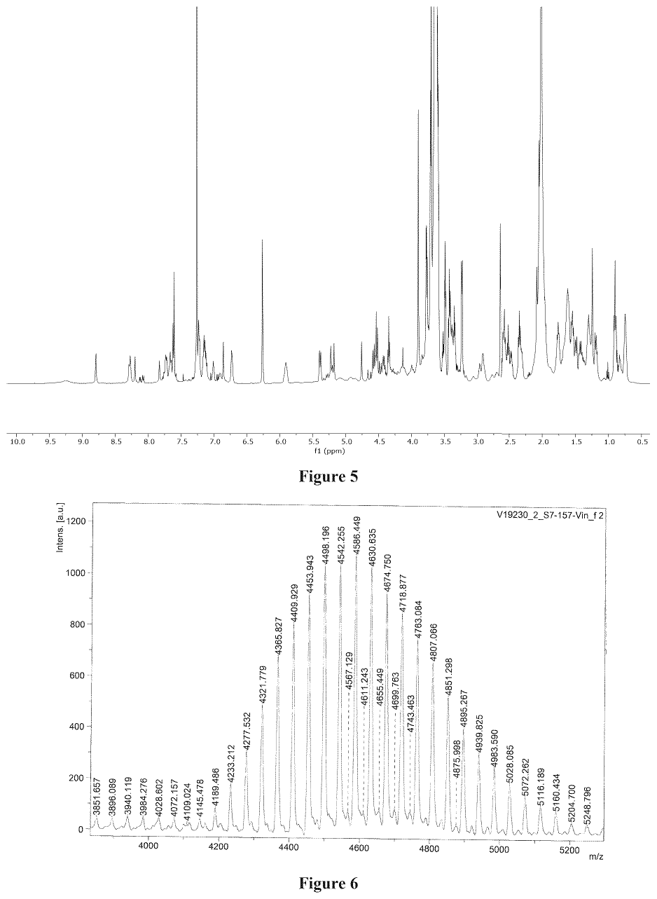

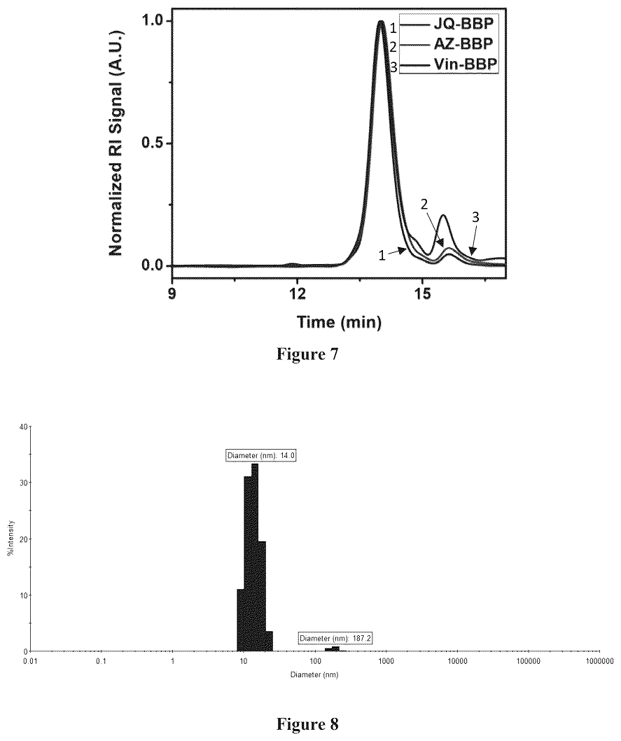

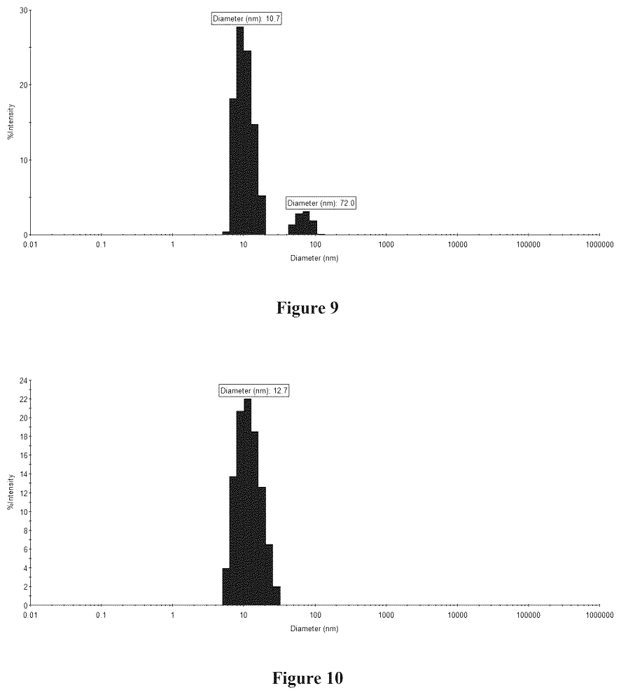

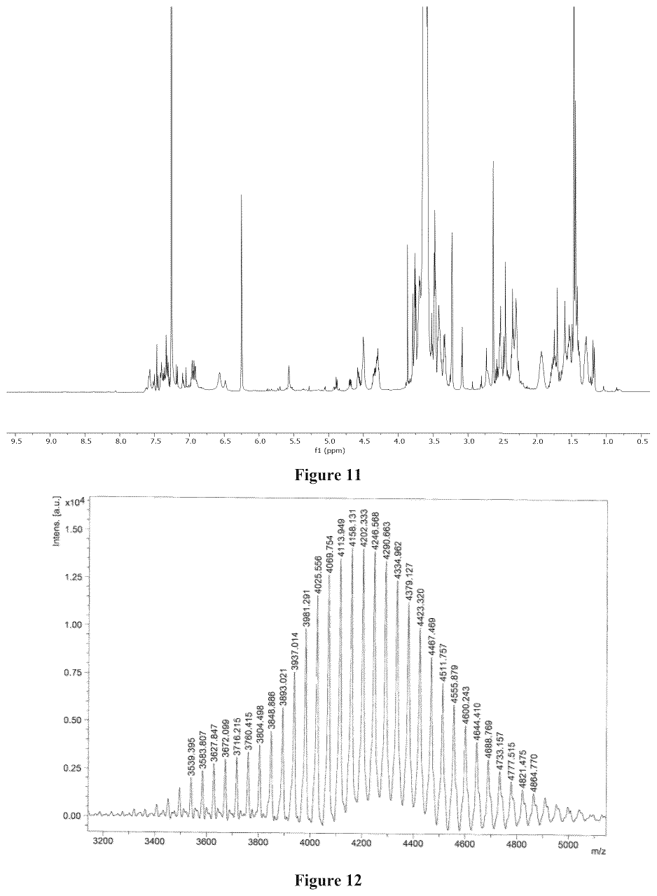

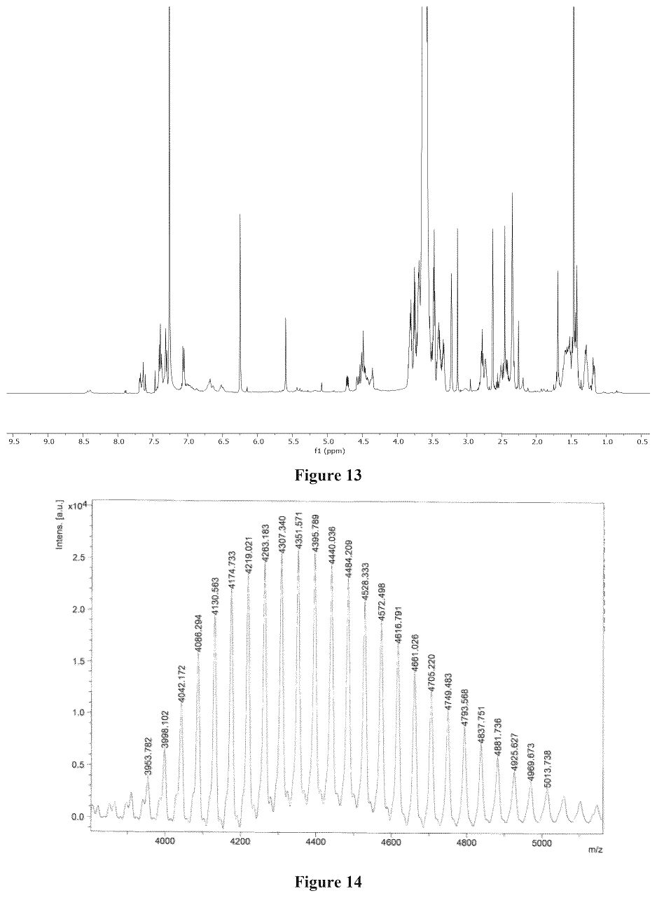

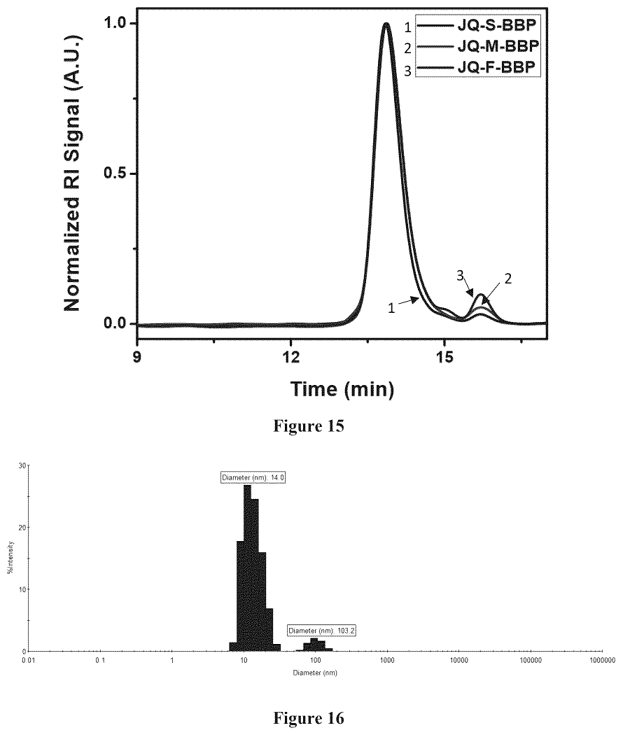

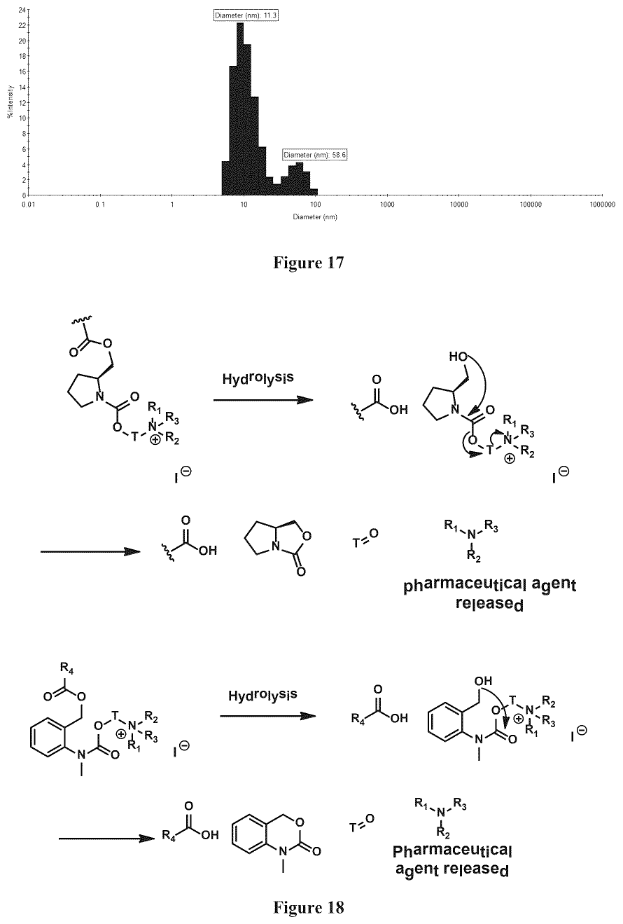

### DETAILED DESCRIPTION OF CERTAIN EMBODIMENTS OF THE DISCLOSURE

The present disclosure provides, in one aspect, Brush prodrugs of pharmaceutical agents (Brush prodrugs). In certain embodiments, the pharmaceutical agents are therapeutic agents, diagnostic agents, and prophylactic agents. The Brush prodrugs may improve the therapeutic index of the pharmaceutical agents, optionally with a favorable biodistribution and release of the pharmaceutical agents in tumor compared to other tissues.

The release (e.g., the rate of release) of the pharmaceutical agents from the Brush prodrugs may be tuned by changing one or more moieties of the Brush prodrugs. In certain embodiments, the release is as shown in the scheme below:

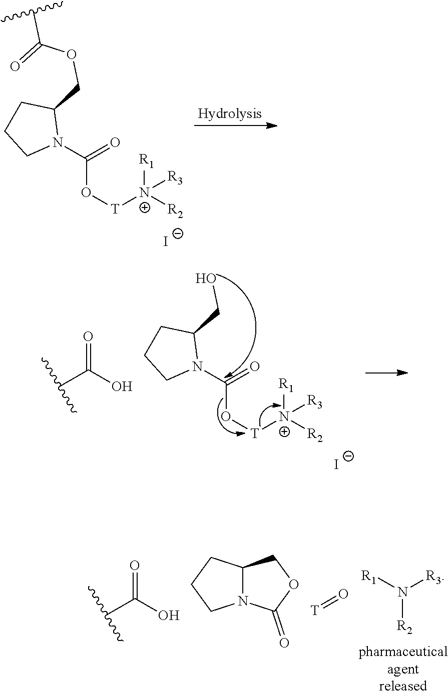

In certain embodiments,

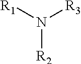

is the pharmaceutical agent, optionally wherein two of R₁, R₂, and R₃ are joined to form a moiety attached to the nitrogen atom through a double bond (e.g., an imine).

In certain embodiments, the release is as shown in the scheme below:

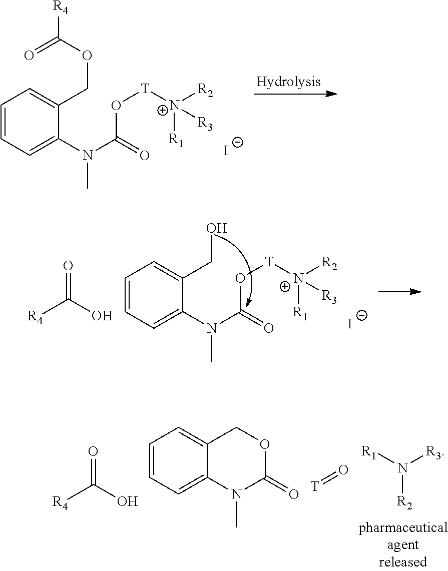

The Brush prodrugs of other pharmaceutical agents are expected to show similar and optionally additional advantages over the free pharmaceutical agents.

### Macromonomers

In one aspect, the present disclosure provides macromonomers of Formula (I):

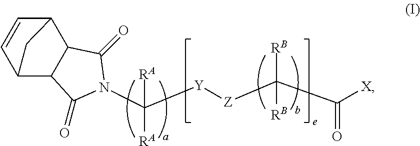

and salts thereof, wherein:
each instance of R^{A} is independently hydrogen, halogen, or substituted or unsubstituted, C₁₋₆ alkyl;
a is an integer from 1 to 20, inclusive;
each instance of —Y—Z— is independently

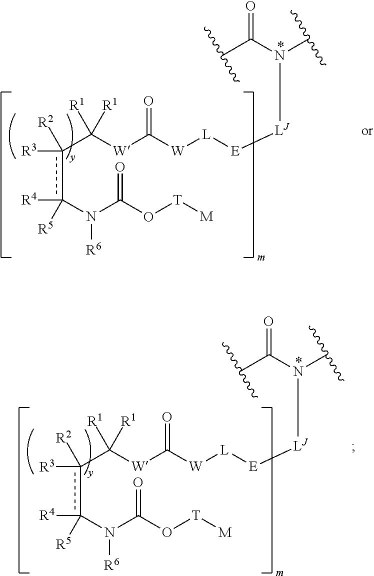

each instance of L is independently substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, or substituted or unsubstituted, C₂₋₂₀₀ heteroalkynylene, wherein:
   optionally one or more carbons in the substituted or unsubstituted, C₁₋₂₀₀ alkylene, substituted or unsubstituted, C₂₋₂₀₀ alkenylene, substituted or unsubstituted, C₂₋₂₀ alkynylene, substituted or unsubstituted, C₂₋₂₀₀ heteroalkylene, substituted or unsubstituted, C₂₋₂₀₀ heteroalkenylene, and substituted or unsubstituted, C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene; and
   optionally one or more heteroatoms in the substituted or unsubstituted, C₂₋₂₀₀ heteroalkylene, substituted or unsubstituted, C₂₋₂₀₀ heteroalkenylene, and substituted or unsubstituted, C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene;
each instance of m is independently an integer from 1 to 10, inclusive;
each instance of E is a moiety formed by reacting E^{A} with E^{B}, each instance of E^{A} is a first reaction handle;
each instance of E^{B} is a second reaction handle, wherein the second reaction handle is able to react with the first reaction handle;
each instance of L is independently substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀ heteroalkenylene, or substituted or unsubstituted, C₂₋₂₀₀ heteroalkynylene, wherein:
   optionally one or more carbons in each instance of the substituted or unsubstituted, C₁₋₂₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, and substituted or unsubstituted, C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene; and
   optionally one or more heteroatoms in each instance of the substituted or unsubstituted, C₂₋₂₀₀ heteroalkylene, substituted or unsubstituted, C₂₋₂₀₀ heteroalkenylene, and substituted or unsubstituted, C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene;
each instance of W is independently a single bond, —O—, —S—, or —NR^{E}—; each instance of R^{E} is independently hydrogen, substituted or unsubstituted, C₁₋₆ alkyl, or a nitrogen protecting group;
each instance of W′ is independently —O— or —S—;
each instance of R¹ is independently hydrogen, halogen, or substituted or unsubstituted, C₁₋₆ alkyl;
each instance of y is independently 0 or 1;
when y is 0, [symbol] is a single bond;
when y is 1, [symbol] is a single or double bond; when [symbol] is a single bond, R² is hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R)₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
each instance of R^{a} is independently hydrogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, an oxygen protecting group when attached to an oxygen atom, a sulfur protecting group when attached to a sulfur atom, or a nitrogen protecting group when attached to a nitrogen atom; or two instances of R^{a} attached to the same nitrogen atom are joined to form substituted or unsubstituted, 3- to 7-membered, monocyclic heterocyclyl, or substituted or unsubstituted, 5- or 6-membered, monocyclic heteroaryl;
when [symbol] is a double bond, R² is absent;
each instance of R³ is independently hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂; each instance of R⁴ is independently hydrogen, halogen, substituted or unsubstituted, C₁₋₆ alkyl, substituted or unsubstituted, C₂₋₆ alkenyl, substituted or unsubstituted, C₂f alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR)N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
or, when y is 1 and [symbol] is a single bond, R³ and R⁴ are joined with their intervening atoms to form substituted or unsubstituted carbocyclyl or substituted or unsubstituted heterocyclyl;
or, when y is 1 and [symbol] is a double bond, R³ and R⁴ are cis to each other and are joined with their intervening atoms to form substituted or unsubstituted carbocyclyl, substituted or unsubstituted heterocyclyl, substituted or unsubstituted aryl, or substituted or unsubstituted heteroaryl;
or R⁴ and R⁶ are joined with their intervening atoms to form substituted or unsubstituted heterocyclyl;
when [symbol] is a single bond, R⁵ is hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR², —NR^{a}C(═O)N(R^{a})₂, —OC(═O)R^{a}, —OC(═O)OR^{a}, or —OC(═O)N(R^{a})₂;
when [symbol] is a double bond, R⁵ is absent;
each instance of R⁶ is independently hydrogen, substituted or unsubstituted, C₁₋₆ alkyl, or a nitrogen protecting group;
each instance of T is independently substituted or unsubstituted methylene; each instance of M is independently an ammonium salt or iminium salt of a pharmaceutical agent, wherein the attachment point is the N⁺ of the ammonium salt or iminium salt;
each instance of R^{B} is independently hydrogen, halogen, or substituted or unsubstituted, C₁₋₆ alkyl;
each instance of b is independently an integer from 1 to 20, inclusive;
e is an integer from 1 to 10, inclusive;
X is OR^{C} or N(R^{D})₂;
R^{C} is hydrogen, substituted or unsubstituted, C₁₋₁₀₀₀ alkyl, substituted or unsubstituted, C₂₋₁₀₀₀ alkenyl, substituted or unsubstituted, C₂₋₁₀₀₀ alkynyl, substituted or unsubstituted, C₁₋₁₀₀₀ heteroalkyl, substituted or unsubstituted, C₂₋₁₀₀ heteroalkenyl, substituted or unsubstituted, C₂₋₁₀₀₀ heteroalkynyl, an oxygen protecting group, or a leaving group; and
each instance of R^{D} is independently hydrogen, substituted or unsubstituted, C₁₋₁₀₀₀ alkyl, substituted or unsubstituted, C₂₋₁₀₀₀ alkenyl, substituted or unsubstituted, C₂₋₁₀₀₀ alkynyl, substituted or unsubstituted, C₁₋₁₀₀₀ heteroalkyl, substituted or unsubstituted, C₂₋₁₀₀₀ heteroalkenyl, substituted or unsubstituted, C₂₋₁₀ heteroalkynyl, or a nitrogen protecting group.

In one aspect, the present disclosure provides macromonomers of Formula (I):

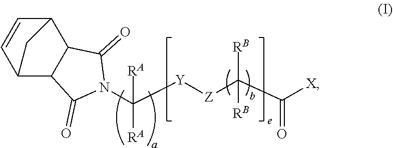

and salts thereof, wherein:
each instance of R^{A} is independently hydrogen, halogen, or substituted or unsubstituted, C₁₋₆ alkyl;
a is an integer from 1 to 20, inclusive;
each instance of —Y—Z— is independently

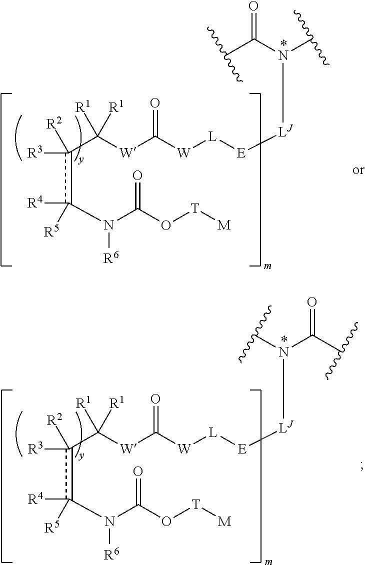

each instance of L is independently substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, or C₂₋₂₀₀ heteroalkynylene, wherein:
   optionally one or more carbons in the substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, and C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene; and
   optionally one or more heteroatoms in the substituted or unsubstituted, C₂₋₂₀₀ heteroalkylene, substituted or unsubstituted, C₂₋₂₀₀ heteroalkenylene, and substituted or unsubstituted, C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene;
each instance of m is independently an integer from 1 to 10, inclusive;
each instance of E is a moiety formed by reacting E^{A} with E^{B}, each instance of E^{A} is a first reaction handle;
each instance of E^{B} is a second reaction handle, wherein the second reaction handle is able to react with the first reaction handle;
each instance of L is independently substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀ heteroalkenylene, or C₂₋₂₀₀ heteroalkynylene, wherein:
   optionally one or more carbons in each instance of the substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, and C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene; and
   optionally one or more heteroatoms in each instance of the substituted or unsubstituted, C₂₋₂₀ heteroalkylene, substituted or unsubstituted, C₂₋₂₀₀ heteroalkenylene, and substituted or unsubstituted, C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene;
each instance of W is independently a single bond, —O—, —S—, or —NR^{E}—;
each instance of R^{E} is independently hydrogen, substituted or unsubstituted, C₁₋₆ alkyl, or a nitrogen protecting group;
each instance of W′ is independently —O— or —S—;
each instance of R¹ is independently hydrogen, halogen, or substituted or unsubstituted, C₁₋₆ alkyl;
each instance of y is independently 0 or 1;
when y is 0, [symbol] is a single bond; when y is 1, [symbol] is a single or double bond;
when [symbol] is a single bond, R² is hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
each instance of R^{a} is independently hydrogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, an oxygen protecting group when attached to an oxygen atom, a sulfur protecting group when attached to a sulfur atom, or a nitrogen protecting group when attached to a nitrogen atom; or two instances of R^{a} attached to the same nitrogen atom are joined to form substituted or unsubstituted, 3- to 7-membered, monocyclic heterocyclyl, or substituted or unsubstituted, 5- or 6-membered, monocyclic heteroaryl;
when [symbol] is double bond, R² is absent;
each instance of R³ is independently hydrogen, halogen, substituted or unsubstituted, C₁ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
each instance of R⁴ is independently hydrogen, halogen, substituted or unsubstituted, C₁₋₆ alkyl, substituted or unsubstituted, C₂₋₆ alkenyl, substituted or unsubstituted, C₂f alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
or, when y is 1 and [symbol] is a single bond, R³ and R⁴ are joined with their intervening atoms to form substituted or unsubstituted carbocyclyl or substituted or unsubstituted heterocyclyl;
or, when y is 1 and [symbol] is a double bond, R³ and R⁴ are cis to each other and are joined with their intervening atoms to form substituted or unsubstituted carbocyclyl, substituted or unsubstituted heterocyclyl, substituted or unsubstituted aryl, or substituted or unsubstituted heteroaryl;
or R⁴ and R⁶ are joined with their intervening atoms to form substituted or unsubstituted heterocyclyl;
when [symbol] is a single bond, R⁵ is hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
when [symbol] is a double bond, R⁵ is absent;
each instance of R⁶ is independently hydrogen, substituted or unsubstituted, C₁₋₆ alkyl, or a nitrogen protecting group;
each instance of T is independently substituted or unsubstituted methylene;
each instance of M is independently an ammonium salt or iminium salt of a pharmaceutical agent, wherein the attachment point is the N⁺ of the ammonium salt or iminium salt;
each instance of R^{B} is independently hydrogen, halogen, or substituted or unsubstituted, C₁₋₆ alkyl;
each instance of b is independently an integer from 1 to 20, inclusive;
e is an integer from 1 to 10, inclusive;
X is OR^{C} or N(R^{D})₂;
R^{C} is hydrogen, substituted or unsubstituted, C₁₋₁₀₀₀ alkyl, substituted or unsubstituted, C₂₋₁₀₀₀ alkenyl, substituted or unsubstituted, C₂₋₁₀₀₀ alkynyl, substituted or unsubstituted, C₁₋₁₀₀₀ heteroalkyl, substituted or unsubstituted, C₂₋₁₀₀₀ heteroalkenyl, substituted or unsubstituted, C₂₋₁₀₀₀ heteroalkynyl, an oxygen protecting group, or a leaving group; and
each instance of R^{D} is independently hydrogen, substituted or unsubstituted, C₁₋₁₀₀₀ alkyl, substituted or unsubstituted, C₂₋₁₀₀₀ alkenyl, substituted or unsubstituted, C₂₋₁₀₀₀ alkynyl, substituted or unsubstituted, C₁₋₁₀₀₀ heteroalkyl, substituted or unsubstituted, C₂₋₁₀₀₀ heteroalkenyl, substituted or unsubstituted, C₂₋₁₀₀₀ heteroalkynyl, or a nitrogen protecting group.

A moiety or variable described in one section of the present disclosure also applies to other sections of the present disclosure, unless otherwise provided.

In certain embodiments, the macromonomer is of the formula:

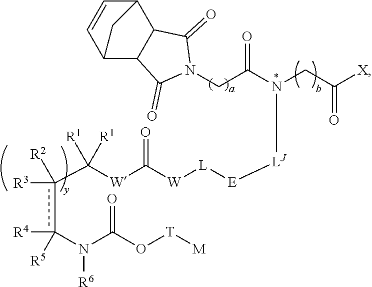

or a salt thereof.

In certain embodiments, the macromonomer is of the formula:

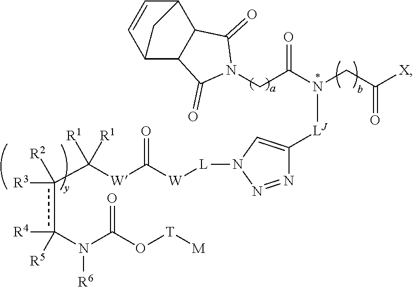

or a salt thereof.

In certain embodiments, the macromonomer is of the formula:

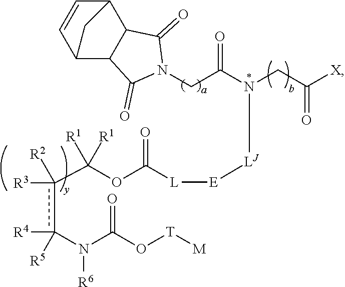

or a salt thereof.

In certain embodiments, the macromonomer is of the formula:

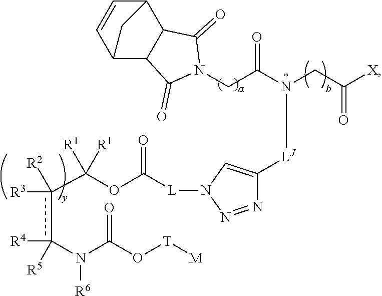

or a salt thereof.

In certain embodiments, the macromonomer is of the formula:

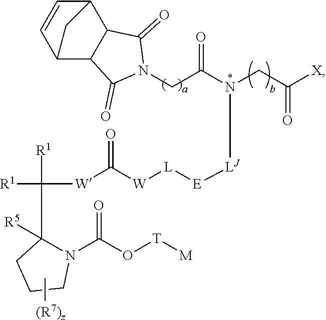

or a salt thereof, wherein:
each instance of R⁷ is independently halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}—, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR², —NR^{a}C(═O)N(R^{a})₂, —OC(═O)R^{a}, —OC(═O)OR^{a}, or —OC(═O)N(R^{a})₂; and
each instance of z is independently 0, 1, 2, 3, 4, 5, or 6.

In certain embodiments, the macromonomer is of the formula:

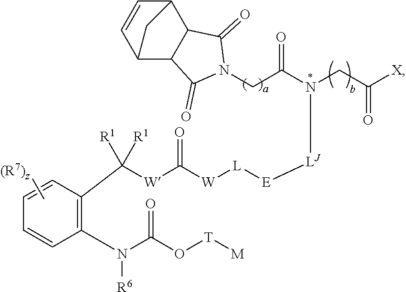

or a salt thereof, wherein:
each instance of R⁷ is independently halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}. —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂; and
each instance of z is independently 0, 1, 2, 3, or 4.

In certain embodiments, z is 0.

When Formula (I) includes two or more instances of a moiety, the two or more instances of the moiety are independent from each other (e.g., any two of them may be the same or different).

In certain embodiments, at least one instance of R^{A} is hydrogen. In certain embodiments, each instance of R^{A} is hydrogen. In certain embodiments, at least one instance of R^{A} is halogen (e.g., F). In certain embodiments, at least one instance of R^{A} is substituted or unsubstituted, C₁₋₆ alkyl (e.g., unsubstituted, C₁₋₆ alkyl, e.g., Me).

In certain embodiments, a is 1. In certain embodiments, a is an integer from 2 to 20, inclusive. In certain embodiments, a is 3, 4, 5, 6, or 7. In certain embodiments, a is 4, 5, or 6. In certain embodiments, a is 5.

In certain embodiments, at least one instance of —Y—Z— is

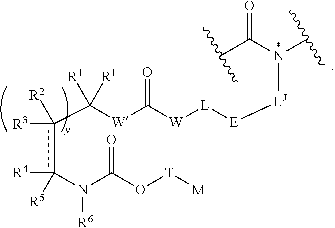

In certain embodiments, at least one instance of —Y—Z— is

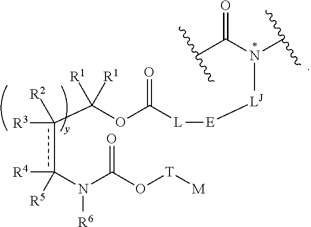

The pharmaceutical agents include chemical compounds and mixtures of chemical compounds, e.g., small organic or inorganic molecules; saccharines; oligosaccharides; polysaccharides; biological macromolecules. e.g., peptides, proteins, and peptide analogs and derivatives; peptidomimetics; antibodies and antigen binding fragments thereof; nucleic acids; nucleic acid analogs and derivatives; an extract made from biological materials such as bacteria, plants, fungi, or animal cells; animal tissues; naturally occurring or synthetic compositions; and any combinations thereof.

In some embodiments, the pharmaceutical agent is a small molecule. In some embodiments, the pharmaceutical agent is a peptide or protein. Exemplary pharmaceutical agents include, but are not limited to, those found in Harrison's 15 Principles of Internal Medicine, 13th Edition, Eds. T. R. Harrison et al. McGraw-Hill N.Y., NY; Physicians' Desk Reference, 50th Edition, 1997, Oradell New Jersey, Medical Economics Co.; Pharmacological Basis of Therapeutics, 8th Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete contents of all of which are incorporated herein by reference.

In certain embodiments, at least one instance of the pharmaceutical agent is a therapeutic agent. In certain embodiments, each instance of the pharmaceutical agent is a therapeutic agent. In some embodiments, exemplary therapeutic agents include, but are not limited to, one or more of the agents listed in Paragraph 0148 of U.S. Pat. No. 9,381,253, incorporated by reference herein. In other embodiments, exemplary therapeutic agents include, but are not limited to, one or more of the therapeutic agents listed in WO 2013/169739, including the anti-hypertensive and/or a collagen modifying agents (“AHCM”) disclosed, e.g., in Paragraphs 40-49, 283, 286-295; the microenviroment modulators disclosed, e.g., in Paragraphs 113-121, of WO 2013/169739, incorporated herein by reference. Examples of therapeutic agents also include, but are not limited to, antimicrobial agents, analgesics, antiinflammatory agents, counterirritants, coagulation modifying agents, diuretics, sympathomimetics, anorexics, antacids and other gastrointestinal agents; antiparasitics, antidepressants, antihypertensives, anticholinergics, stimulants, antihormones, central and respiratory stimulants, drug antagonists, lipid-regulating agents, uricosurics, cardiac glycosides, electrolytes, ergot and derivatives thereof, expectorants, hypnotics and sedatives, antidiabetic agents, dopaminergic agents, antiemetics, muscle relaxants, para-sympathomimetics, anticonvulsants, antihistamines, beta-blockers, purgatives, antiarrhythmics, contrast materials, radiopharmaceuticals, antiallergic agents, tranquilizers, vasodilators, antiviral agents, and antineoplastic or cytostatic agents or other agents with anticancer properties, or a combination thereof. Other suitable therapeutic agents include contraceptives and vitamins as well as micro- and macronutrients. Still other examples include antiinfectives such as antibiotics and antiviral agents; analgesics and analgesic combinations; anorexics; antiheimintics; antiarthritics; antiasthmatic agents; anticonvulsants; antidepressants; antidiuretic agents; antidiarrheals; antihistamines; antiinflammatory agents; antimigraine preparations; antinauseants; antineoplastics; antiparkinsonism drugs; antipruritics; antipsychotics; antipyretics, antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary, peripheral and cerebral; central nervous system stimulants; cough and cold preparations, including decongestants; hormones such as estradiol and other steroids, including corticosteroids; hypnotics; immunosuppressives; muscle relaxants; parasympatholytics; psychostimulants; sedatives; and tranquilizers; and naturally derived or genetically engineered proteins, polysaccharides, glycoproteins, or lipoproteins. In certain embodiments, at least one instance of the therapeutic agent is an anti-cancer agent. Anti-cancer agents encompass biotherapeutic anti-cancer agents as well as chemotherapeutic agents. Exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g., HERCEPTIN (trastuzumab), T-DM1, AVASTIN (bevacizumab), ERBITUX (cetuximab), VECTIBIX (panitumumab), RITUXAN (rituximab), BEXXAR (tositumomab)). Exemplary chemotherapeutic agents include, but are not limited to, anti-estrogens (e.g., tamoxifen, raloxifene, and megestrol), LHRH agonists (e.g., goscrclin and leuprolide), anti-androgens (e.g., flutamide and bicalutamide), photodynamic therapies (e.g., vertoporfin (BPD-MA), phthalocyanine, photosensitizer Pc4, and demethoxy-hypocrellin A (2BA-2-DMHA)), nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g., dacarbazine, temozolomide), platinum containing compounds (e.g., cisplatin, carboplatin, oxaliplatin), vinca alkaloids (e.g., vincristine, vinblastine, vindesine, and vinorelbine), taxoids (e.g., paclitaxel or a paclitaxel equivalent) docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g., etoposide, etoposide phosphate, teniposide, topotecan, 9-aminocamptothecin, camptoirinotecan, irinotecan, crisnatol, mytomycin C), anti-metabolites, DHFR inhibitors (e.g., methotrexate, dichloromethotrexate, trimetrexate, edatrexate), IMP dehydrogenase inhibitors (e.g., mycophenolic acid, tiazofurin, ribavirin, and EICAR), ribonucleotide reductase inhibitors (e.g., hydroxyurea and deferoxamine), uracil analogs (e.g., 5-fluorouracil (5-FU), floxuridine, doxifluridine, raltitrexed, tegafur-uracil, capecitabine), cytosine analogs (e.g., cytarabine (ara C), cytosine arabinoside, and fludarabine), purine analogs (e.g., mercaptopurine and Thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone), MDR inhibitors (e.g., verapamil), Ca² ATPase inhibitors (e.g., thapsigargin), imatinib, thalidomide, lenalidomide, tyrosine kinase inhibitors (e.g., axitinib (AGO13736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe), and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine. In certain embodiments, the anti-cancer agent is JQ1, AZD5153, vincristine, abiraterone acetate (e.g., ZYTIGA), ABVD, ABVE, ABVE-PC, AC, AC-T, ADE, ado-trastuzumab emtansine (e.g., KADCYLA), afatinib dimaleate (e.g., GILOTRIF), aldesleukin (e.g., PROLEUKIN), alemtuzumab (e.g., CAMPATH), anastrozole (e.g., ARIMIDEX), arsenic trioxide (e.g., TRISENOX), asparaginase Erwinia chrysanthemi (e.g., ERWINAZE), axitinib (e.g., INLYTA), azacitidine (e.g., MYLOSAR, VIDAZA), BEACOPP, belinostat (e.g., BELEODAQ), bendamustine hydrochloride (e.g., TREANDA), BEP, bevacizumab (e.g., AVASTIN), bicalutamide (e.g., CASODEX), bleomycin (e.g., BLENOXANE), blinatumomab (e.g., BLINCYTO), bortezomib (e.g., VELCADE), bosutinib (e.g., BOSULIF), brentuximab vedotin (e.g., ADCETRIS), busulfan (e.g., BUSULFEX, MYLERAN), cabazitaxel (e.g., JEVTANA), cabozantinib-s-malate (e.g., COMETRIQ), CAF, capecitabine (e.g., XELODA), CAPOX, carboplatin (e.g., PARAPLAT, PARAPLATIN), carboplatin-taxol, carfilzomib (e.g., KYPROLIS), carmustine (e.g., BECENUM, BICNU, CARMUBRIS), carmustine implant (e.g., GLIADEL WAFER, GLIADEL), ceritinib (e.g., ZYKADIA), cetuximab (e.g., ERBITUX), chlorambucil (e.g., AMBOCHLORIN, AMBOCLORIN, LEUKERAN, LINFOLIZIN), chlorambucil-prednisone, CHOP, cisplatin (e.g., PLATINOL, PLATINOL-AQ), clofarabine (e.g., CLOFAREX, CLOLAR), CMF, COPP, COPP-ABV, crizotinib (e.g., XALKORI), CVP, cyclophosphamide (e.g., CLAFEN, CYTOXAN, NEOSAR), cytarabine (e.g., CYTOSAR-U, TARABINE PFS), dabrafenib (e.g., TAFINLAR), dacarbazine (e.g., DTIC-DOME), dactinomycin (e.g., COSMEGEN), dasatinib (e.g., SPRYCEL), daunorubicin hydrochloride (e.g., CERUBIDINE), decitabine (e.g., DACOGEN), degarelix, denileukin diftitox (e.g., ONTAK), denosumab (e.g., PROLIA, XGEVA), Dinutuximab (e.g., UNITUXIN), docetaxel (e.g., TAXOTERE), doxorubicin hydrochloride (e.g., ADRIAMYCIN PFS, ADRIAMYCIN RDF), doxorubicin hydrochloride liposome (e.g., DOXIL, DOX-SL, EVACET, LIPODOX), enzalutamide (e.g., XTANDI), epirubicin hydrochloride (e.g., ELLENCE), EPOCH, erlotinib hydrochloride (e.g., TARCEVA), etoposide (e.g., TOPOSAR, VEPESID), etoposide phosphate (e.g., ETOPOPHOS), everolimus (e.g., AFINITOR DISPERZ, AFINITOR), exemestane (e.g., AROMASIN), FEC, fludarabine phosphate (e.g., FLUDARA), fluorouracil (e.g., ADRUCIL, EFUDEX, FLUOROPLEX), FOLFIRI, FOLFIRI-BEVACIZUMAB, FOLFIRI-CETUXIMAB, FOLFIRINOX, FOLFOX, FU-LV, fulvestrant (e.g., FASLODEX), gefitinib (e.g., IRESSA), gemcitabine hydrochloride (e.g., GEMZAR), gemcitabine-cisplatin, gemcitabine-oxaliplatin, goserelin acetate (e.g., ZOLADEX), Hyper-CVAD, ibritumomab tiuxetan (e.g., ZEVALIN), ibrutinib (e.g., IMBRUVICA), ICE, idelalisib (e.g., ZYDELIG), ifosfamide (e.g., CYFOS, IFEX, IFOSFAMIDUM), imatinib mesylate (e.g., GLEEVEC), imiquimod (e.g., ALDARA), ipilimumab (e.g., YERVOY), irinotecan hydrochloride (e.g., CAMPTOSAR), ixabepilone (e.g., IXEMPRA), lanreotide acetate (e.g., SOMATULINE DEPOT), lapatinib ditosylate (e.g., TYKERB), lenalidomide (e.g., REVLIMID), lenvatinib (e.g., LENVIMA), letrozole (e.g., FEMARA), leucovorin calcium (e.g., WELLCOVORIN), leuprolide acetate (e.g., LUPRON DEPOT, LUPRON DEPOT-3 MONTH, LUPRON DEPOT-4 MONTH, LUPRON DEPOT-PED, LUPRON, VIADUR), liposomal cytarabine (e.g., DEPOCYT), lomustine (e.g., CEENU), mechlorethamine hydrochloride (e.g., MUSTARGEN), megestrol acetate (e.g., MEGACE), mercaptopurine (e.g., PURINETHOL, PURIXAN), methotrexate (e.g., ABITREXATE, FOLEX PFS, FOLEX, METHOTREXATE LPF, MEXATE, MEXATE-AQ), mitomycin c (e.g., MITOZYTREX, MUTAMYCIN), mitoxantrone hydrochloride, MOPP, nelarabine (e.g., ARRANON), nilotinib (e.g., TASIGNA), nivolumab (e.g., OPDIVO), obinutuzumab (e.g., GAZYVA), OEPA, ofatumumab (e.g., ARZERRA), OFF, olaparib (e.g., LYNPARZA), omacetaxine mepesuccinate (e.g., SYNRIBO), OPPA, OTX-015, oxaliplatin (e.g., ELOXATIN), paclitaxel (e.g., TAXOL), paclitaxel albumin-stabilized nanoparticle formulation (e.g., ABRAXANE), PAD, palbociclib (e.g., IBRANCE), pamidronate disodium (e.g., AREDIA), panitumumab (e.g., VECTIBIX), panobinostat (e.g., FARYDAK), pazopanib hydrochloride (e.g., VOTRIENT), pegaspargase (e.g., ONCASPAR), peginterferon alfa-2b (e.g., PEG-INTRON), peginterferon alfa-2b (e.g., SYLATRON), pembrolizumab (e.g., KEYTRUDA), pemetrexed disodium (e.g., ALIMTA), pertuzumab (e.g., PERJETA), plerixafor (e.g., MOZOBIL), pomalidomide (e.g., POMALYST), ponatinib hydrochloride (e.g., ICLUSIG), pralatrexate (e.g., FOLOTYN), prednisone, procarbazine hydrochloride (e.g., MATULANE), radium 223 dichloride (e.g., XOFIGO), raloxifene hydrochloride (e.g., EVISTA, KEOXIFENE), ramucirumab (e.g., CYRAMZA), R-CHOP, recombinant HPV bivalent vaccine (e.g., CERVARIX), recombinant human papillomavirus (e.g., HPV) nonavalent vaccine (e.g., GARDASIL 9), recombinant human papillomavirus (e.g., HPV) quadrivalent vaccine (e.g., GARDASIL), recombinant interferon alfa-2b (e.g., INTRON A), regorafenib (e.g., STIVARGA), rituximab (e.g., RITUXAN), romidepsin (e.g., ISTODAX), ruxolitinib phosphate (e.g., JAKAFI), siltuximab (e.g., SYLVANT), sipuleucel-t (e.g., PROVENGE), sorafenib tosylate (e.g., NEXAVAR), STANFORD V, sunitinib malate (e.g., SUTENT), TAC, tamoxifen citrate (e.g., NOLVADEX, NOVALDEX), temozolomide (e.g., METHAZOLASTONE, TEMODAR), temsirolimus (e.g., TORISEL), thalidomide (e.g., SYNOVIR, THALOMID), thiotepa, topotecan hydrochloride (e.g., HYCAMTIN), toremifene (e.g., FARESTON), tositumomab and iodine 1131 tositumomab (e.g., BEXXAR), TPF, trametinib (e.g., MEKINIST), trastuzumab (e.g., HERCEPTIN), VAMP, vandetanib (e.g., CAPRELSA), VEIP, vemurafenib (e.g., ZELBORAF), vinblastine sulfate (e.g., VELBAN, VELSAR), vincristine sulfate (e.g., VINCASAR PFS), vincristine sulfate liposome (e.g., MARQIBO), vinorelbine tartrate (e.g., NAVELBINE), vismodegib (e.g., ERIVEDGE), vorinostat (e.g., ZOLINZA), XELIRI, XELOX, ziv-aflibercept (e.g., ZALTRAP), or zoledronic acid (e.g., ZOMETA), or a pharmaceutically acceptable salt thereof. In certain embodiments, at least one instance of the therapeutic agent is a bromodomain inhibitor. In certain embodiments, at least one instance of the therapeutic agent is a bromo and extra terminal protein (BET) inhibitor. In certain embodiments, at least one instance of the therapeutic agent is a bromodomain-containing protein 2 (BRD2) inhibitor, bromodomain-containing protein 3 (BRD3) inhibitor, bromodomain-containing protein 4 (BRD4) inhibitor, TBP (TATA box binding protein)-associated factor protein (TAF) (e.g., TAF1 or TAF1L) inhibitor, CREB-binding protein (CBP) inhibitor, or E1A binding protein p300 (EP300) inhibitor. In certain embodiments, at least one instance of the pharmaceutical agent is a PARP inhibitor, ALK inhibitor, or STING ligand. In certain embodiments, at least one instance of the therapeutic agent is OTX-015.

In certain embodiments, the therapeutic agent is JQ1, AZD5153, or vincristine. In certain embodiments, at least one instance of M is

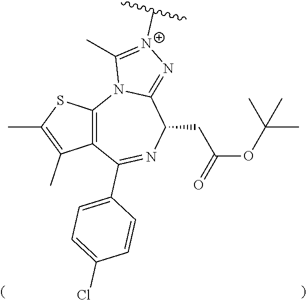 [SMILES: *[n+]1nc2n(c1C)-c1sc(C)c(C)c1C(c1ccc(Cl)cc1)=N[C@H]2CC(=O)OC(C)(C)C]

(an anionic counterion),

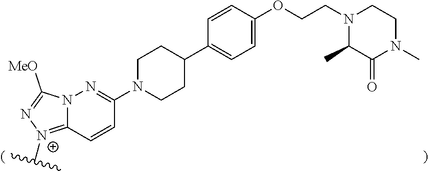 [SMILES: *[n+]1nc(OC)n2nc(N3CCC(c4ccc(OCCN5CCN(C)C(=O)[C@H]5C)cc4)CC3)ccc21]

an anionic counterion), or

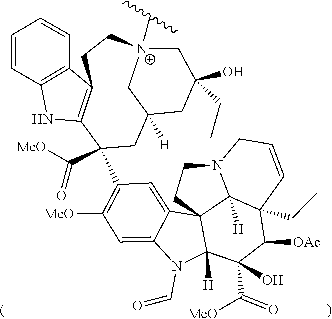

(an anionic counterion).

In certain embodiments, at least one instance of the pharmaceutical agent is a prophylactic agent. In certain embodiments, each instance of the pharmaceutical agent is a prophylactic agent. Prophylactic agents that can be included in the conjugates of the invention include, but are not limited to, antibiotics, nutritional supplements, and vaccines. Vaccines may comprise isolated proteins or peptides, inactivated organisms and viruses, dead organisms and viruses, genetically altered organisms or viruses, and cell extracts. Prophylactic agents may be combined with interleukins, interferon, cytokines, and adjuvants such as cholera toxin, alum, Freund's adjuvant.

In certain embodiments, at least one instance of the pharmaceutical agent is a diagnostic agent. In certain embodiments, each instance of the pharmaceutical agent is a diagnostic agent. Exemplary diagnostic agents include, but are not limited to, fluorescent molecules; gases; metals; imaging agents, such as commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium. Examples of materials useful for CAT and x-ray imaging include iodine-based materials. In certain embodiments, the diagnostic agent is used in magnetic resonance imaging (MRI), such as iron oxide particles or gadolinium complexes. Gadolinium complexes that have been approved for clinical use include gadolinium chelates with DTPA, DTPA-BMA, DOTA and HP-DO3A which are reviewed in Aime, et al. (Chemical Society Reviews (1998), 27:19-29), the entire teachings of which are incorporated herein by reference.

In certain embodiments, the diagnostic agent is a metal, inorganic compound, organometallic compound, organic compound, or salt thereof. In certain embodiments, the imaging agent contains a metal selected from the group consisting of scandium, titanium, vanadium, chromium, manganese, iron, cobalt, nickel, copper, zinc, yttrium, zirconium, niobium, molybdenum, technetium, ruthenium, rhodium, palladium, silver, cadmium, hafnium, tantalum, tungsten, rhenium, osmium, iridium, platinum, gold, mercury, rutherfordium, dubnium, seaborgium, bohrium, hassium, meitnerium, gadolinium, gallium, thallium, and barium. In certain embodiments, the diagnostic agent is an organic compound. In certain embodiments, the diagnostic agent is metal-free. In certain embodiments, the diagnostic agent is a metal-free organic compound.

In certain embodiments, the imaging agent is a magnetic resonance imaging (MRI) agent. In certain embodiments, the MRI agent is gadolinium. In certain embodiments, the MRI agent is a nitroxide radical-containing compound.

In certain embodiments, the imaging agent is a nuclear medicine imaging agent. In certain embodiments, the nuclear medicine imaging agent is selected from the group consisting of ⁶⁴Cu diacetyl-bis(N⁴-methylthiosemicarbazone) (⁶⁴Cu-ASTM), ¹⁸F-fluorodeoxyglucose (FDG), ¹⁸F-fluoride, 3′-deoxy-3′-[¹⁸F]fluorothymidine (FLT), ¹⁸F-fluoromisonidazole (FMISO), gallium, technetium-99m, and thallium.

In certain embodiments, the imaging agent is radiographic imaging agent. In certain embodiments, the radiographic imaging agent is selected from the group consisting of barium, gastrografin, and iodine contrast agent.

In certain embodiments, the imaging agent the diagnostic agent is a radical-containing compound. In certain embodiments, the imaging agent is a nitroxide radical-containing compound. In certain embodiments, the imaging agent the diagnostic agent is of the formula:

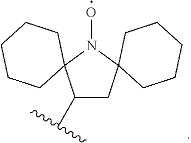

In certain embodiments, the imaging agent the diagnostic agent is an organic compound. In certain embodiments, the imaging agent is a salt of an organic compound. In certain embodiments, the imaging agent the diagnostic agent is of the formula:

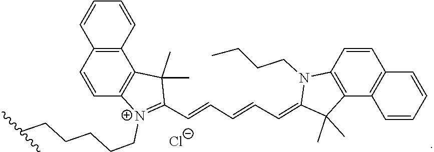 [SMILES: *CCCCC[N+]1=C(/C=C/C=C/C=C2\N(CCCC)c3ccc4ccccc4c3C2(C)C)C(C)(C)c2c1ccc1ccccc21 ; [Cl-]]

In certain embodiments, the diagnostic agent may comprise a fluorescent molecule, a metal chelate, a contrast agent, a radionuclide, or a positron emission tomography (PET) imaging agent, an infrared imaging agent, a near-IR imaging agent, a computer assisted tomography (CAT) imaging agent, a photon emission computerized tomography imaging agent, an X-ray imaging agent, or a magnetic resonance imaging (MRI) agent.

In some embodiments, the diagnostic agent is a fluorescent molecule. In some embodiments, the fluorescent molecule comprises an acridine dye, a cyanine dye, a rhodamine dye, a BODIPY dye, a fluorescein dye, a dansyl dye, an Alexa dye, an atto dye, a quantum dot, or a fluorescent protein. In some embodiments, the fluorescent molecule is a cyanine dye (e.g., Cy3, Cy 3.5, Cy5, Cy5.5, Cy7, or Cy7.5).

In some embodiments, the diagnostic agent is an MRI agent (e.g., a contrast agent). Examples of suitable materials for use as MRI agents (e.g., contrast agents) include gadolinium chelates, as well as iron, magnesium, manganese, copper, and chromium.

In some embodiments, the diagnostic agent is a CAT imaging agent or an X-ray imaging agent. Examples of materials useful for CAT and X-ray imaging include iodine-based materials.

In some embodiments, the diagnostic agent is a PET imaging agent. Examples of suitable PET imaging agents include compounds and compositions comprising the positron emitting radioisotopes ¹⁸F, ¹⁵O, ¹³N, ¹¹C, ⁸²Rb, ⁶⁴Cu, and ⁶⁸Ga, e.g., fludeoxyglucose (¹⁸F-FDG), (Ga-DOTA-pseudopeptides (e.g., ⁶⁸Ga-DOTA-TOC), ¹¹C-metomidate. ¹¹C-acetate. ¹¹C-methionine, ¹¹C-choline, ¹⁸F-fluciclovine, ¹⁸F-fluorocholine, ¹⁸F-fluorodeoxysorbitol, ¹⁸F-3′-fluoro-3′-deoxythymidine, ¹¹C-raclopride, and ¹⁸F-desmethoxyfallypride.

In some embodiments, the diagnostic agent is a near-IR imaging agent. Examples of near-IR imaging agents include Pz 247, DyLight 750, DyLight 800, cyanine dyes (e.g., Cy5, Cy5.5, Cy7), AlexaFluor 680, AlexaFluor 750, IRDye 680, IRDye 800CW, and Kodak X-SIGHT dyes.

In some embodiments, the agent can be a radionuclide, e.g., for use as a therapeutic, diagnostic, or prognostic agents. Among the radionuclides used, gamma-emitters, positron-emitters, and X-ray emitters are suitable for diagnostic and/or therapy, while beta emitters and alpha-emitters may also be used for therapy. Suitable radionuclides for forming use with various embodiments of the present invention include, but are not limited to, ¹²³I, ¹²⁵I, ¹³⁰I, ¹³¹I, ¹³³I, ¹³⁵I, ⁴⁷Sc, ⁷²As, ⁷²Sc, ⁹⁰Y, ⁸⁸Y, ⁹⁷Ru, ¹⁰⁰Pd, ^{101m}Rh, ¹¹⁹Sb, ¹²⁸Ba, ¹⁹⁷Hg, ²¹¹At, ²¹²Bi, ²¹²Pb, ¹⁰⁹Pd, ¹¹¹In, ⁶⁷Ga, ⁶⁸Ga, ⁶⁷Cu, ⁷⁵Br, ⁷⁷Br, ^{99m}Tc, ¹⁴C, ¹³N, ¹⁵O, ³²P, ³³P, or ¹⁸F.

In certain embodiments, at least one instance of the diagnostic agent is a contrast agent. In certain embodiments, at least one instance of the contrast agent is a magnetic-resonance signal enhancing agent, X-ray attenuating agent, ultrasound scattering agent, or ultrasound frequency shifting agent.

In certain embodiments, the pharmaceutical agent is a monovalent radical. In certain embodiments, the monovalent radical of the pharmaceutical agent is formed by removing a hydrogen atom from the moiety HV of the pharmaceutical agent. In certain embodiments, V is a carbon atom. In certain embodiments, V is a heteroatom. In certain embodiments, V is an oxygen atom. In certain embodiments, V is a sulfur atom. In certain embodiments, V is a nitrogen atom. In certain embodiments, the monovalent radical of the pharmaceutical agent is formed further by changing the atom V of the pharmaceutical agent to substituted or unsubstituted U, wherein each of V and U is a heteroatom, and V and U are different from each other.

In certain embodiments, M is an ammonium (e.g., a quaternary ammonium) salt or iminium (e.g., tertiary iminium) salt of the pharmaceutical agent, wherein the attachment point is the N⁺ of the ammonium salt or iminium salt. In certain embodiments, the nitrogen atom of the N⁺ of the ammonium salt or iminium salt is part of the pharmaceutical agent.

In certain embodiments, at least one instance of the pharmaceutical agent comprises a tertiary amino or secondary imine. In certain embodiments, at least one instance of the pharmaceutical agent does not comprise —OH, —SH, —NH—, —NH₂, or ═NH.

In certain embodiments, M is electrically neutral. In certain embodiments, the macromolecule, compound, or conjugate is electrically neutral.

In certain embodiments, all instances of M are the same. In certain embodiments, at least two instances of M (e.g., all instances of M) are different from each other.

In certain embodiments, at least one instance of m is 1. In certain embodiments, each instance of m is 1. In certain embodiments, at least one instance of m is an integer from 2 to 10, inclusive. In certain embodiments, at least one instance of m is 2, 3, 4, or 5.

When a first divalent moiety comprises a second divalent moiety, the second divalent moiety is part of the backbone of the first divalent moiety. For example, when L^{F} comprises —S—S—, —S—S— is part of the backbone of L^{F}.

In certain embodiments, at least one instance of L is substituted or unsubstituted, C₁₋₂₀₀ alkylene. In certain embodiments, at least one instance of L is unsubstituted C₂₋₁₀ alkylene. In certain embodiments, each instance of L is unsubstituted C₂₋₁₀ alkylene. In certain embodiments, at least one instance of L is unsubstituted C₂₋₂₀ alkylene. In certain embodiments, each instance of L is unsubstituted C₂₋₂₀ alkylene. In certain embodiments, at least one instance of L is substituted or unsubstituted, C₂₋₂₀ alkylene. In certain embodiments, at least one instance of L is substituted or unsubstituted, C₃₋₃₀ alkylene. In certain embodiments, at least one instance of L is substituted or unsubstituted, C₂₋₂₀₀ alkenylene. In certain embodiments, at least one instance of L is substituted or unsubstituted, C₂₋₂₀₀ alkynylene. In certain embodiments, at least one instance of L is substituted or unsubstituted, C₂₋₂₀₀ heteroalkylene. In certain embodiments, at least one instance of L is substituted or unsubstituted, C₂₋₂₀ heteroalkylene. In certain embodiments, at least one instance of L is substituted or unsubstituted, C₃₋₃₀ heteroalkylene. In certain embodiments, at least one instance of L is —CH₂CH₂—(O—CH₂CH₂)₁₋₆—. In certain embodiments, each instance of L is —CH₂CH₂—(O—CH₂CH₂)₁₋₆—. In certain embodiments, at least one instance of L is unsubstituted C₂₋₂₀ alkylene or —CH₂CH₂—(O—CH₂CH₂)₁₋₆—. In certain embodiments, each instance of L is independently unsubstituted C₂₋₂₀ alkylene or —CH₂CH₂—(O—CH₂CH₂)₁₋₆—. In certain embodiments, each instance of L is unsubstituted C₂₋₂₀ alkylene or —CH₂CH₂—(O—CH₂CH₂)₁₋₆—. In certain embodiments, at least one instance of L is substituted or unsubstituted, C₂₋₂₀₀ heteroalkenylene. In certain embodiments, at least one instance of L is substituted or unsubstituted, C₂₋₂₀ heteroalkynylene. In certain embodiments, at least one instance of L is substituted or unsubstituted, C₂₋₂₀₀ heteroalkylene, wherein one or more carbons and/or one or more heteroatoms, of the substituted or unsubstituted, C₂₋₂₀₀ heteroalkylene, are independently replaced with substituted or unsubstituted heteroarylene. In certain embodiments, at least one instance of L is substituted or unsubstituted, C₂₋₂₀₀ heteroalkylene (e.g., substituted or unsubstituted, C₃₋₃₀ heteroalkylene), wherein one or two carbons and/or one or two heteroatoms, of the substituted or unsubstituted, C₂₋₂₀₀ heteroalkylene (e.g., substituted or unsubstituted, C₃₋₃₀ heteroalkylene) are independently replaced with substituted or unsubstituted arylene (e.g., phenylene) or substituted or unsubstituted heteroarylene (e.g., substituted or unsubstituted, monocyclic, 5- or 6-membered heteroarylene). In certain embodiments, at least one instance of L is substituted or unsubstituted, C₂₋₂₀₀ heteroalkylene, wherein one or more carbons and/or one or more heteroatoms, of the substituted or unsubstituted, C₂₋₂₀₀ heteroalkylene, are independently replaced with

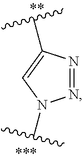 [SMILES: *c1cn(*)nn1]

herein the nitrogen atom labeled with “*” is closer to the attachment point labeled with “**” than the attachment point labeled with “***”. In certain embodiments, at least one instance of L is substituted or unsubstituted, C₂₋₂₀₀ heteroalkylene, wherein one carbon or one heteroatom, of the substituted or unsubstituted, C₂₋₂₀₀ heteroalkylene, is replaced with

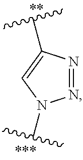 [SMILES: *c1cn(*)nn1]

wherein the nitrogen atom labeled with “*” is closer to the attachment point labeled with “**” than the attachment point labeled with “***”. In certain embodiments, at least one instance of L comprises

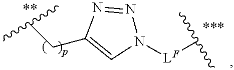

wherein:
each instance of p is independently an integer from 1 to 10, inclusive;
each instance of L^{F} is independently substituted or unsubstituted, C₂₋₁₈₀ heteroalkylene; and
the nitrogen atom labeled with “*” is closer to the attachment point labeled with “**” than the attachment point labeled with “***”. In certain embodiments, at least one instance of L is

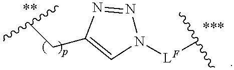

In certain embodiments, at least one instance of L comprises

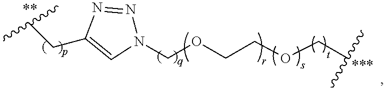

wherein:
each instance of p is independently an integer from 1 to 10, inclusive;
each instance of q is independently an integer from 1 to 10, inclusive;
each instance of r is independently an integer from 0 to 10, inclusive;
each instance of s is independently 0 or 1;
each instance of t is independently an integer from 0 to 10, inclusive; and the nitrogen atom labeled with “*” is closer to the attachment point labeled with “**” than the attachment point labeled with “***”.

In certain embodiments, at least one instance of L^{F} is substituted or unsubstituted, C₃₋₃₀ heteroalkylene. In certain embodiments, at least one instance of L^{F} comprises —S— S—. In certain embodiments, at least one instance of L^{F} is substituted or unsubstituted, C₃₋₃₀ heteroalkylene comprising one —S—S— and no other heteroatoms in the backbone. In certain embodiments, at least one instance of L^{F} comprises a peptide comprising between 1 and 20 (e.g., between 1 and 4), inclusive, amino acid residues.

In certain embodiments, at least one instance of L is

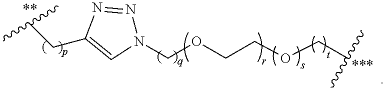

In certain embodiments, at least one instance of L is

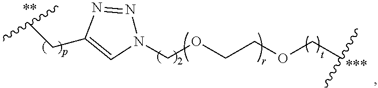

wherein r is 1, 2, or 3; and t is 1 or 2. In certain embodiments, at least one instance of L comprises

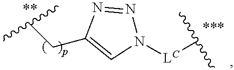

wherein
each instance of p is independently an integer from 1 to 10, inclusive;
each instance of L^{C} is independently substituted or unsubstituted, C₁₋₁₈₀ alkylene; and the nitrogen atom labeled with “*” is closer to the attachment point labeled with “*8” than the attachment point labeled with “***”. In certain embodiments, at least one instance of L is

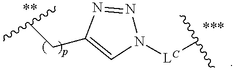

In certain embodiments, at least one instance of L^{C} is substituted or unsubstituted, C₁₋₁₂ alkylene. In certain embodiments, at least one instance of L^{C} is unsubstituted C₁₋₁₂ alkylene. In certain embodiments, each instance of L^{C} is independently C₁₋₁₈₀ alkylene substituted with one or more instances of: substituted or unsubstituted phenyl and/or substituted or unsubstituted, C₁₋₆ alkyl. In certain embodiments, at least one instance of L comprises a polymer. In certain embodiments, at least one instance of the polymer is substituted or unsubstituted polyethylene (e.g., unsubstituted polystyrene). In certain embodiments, the weight-average molecular weight of at least one instance of the polymer is between 300 and 10,000, between 300 and 3,000, between 300 and 1,000, between 1,000 and 10,000, between 1,000 and 3,000, or between 3,000 and 10,000, inclusive, g/mol. In certain embodiments, at least one instance of L comprises an amino acid or a peptide. In certain embodiments, at least one instance the peptide consists of between 3 and 60, between 3 and 30, between 3 and 10, between 10 and 60, between 10 and 30, or between 30 and 60, inclusive, amino acids. In certain embodiments, each instance of the amino acid is a natural amino acid. In certain embodiments, at least one instance of the amino acid is an unnatural amino acid.

A cleavable linker is “cleaved” or “degraded” when one or more bonds of the cleavable linker are broken. e.g., resulting in release of an agent, e.g., from the Brush prodrug or particle. Linker cleavage or agent release need not be 100%, e.g., a cleavage or release of at least 10%, 20%, 30%, 40%, 50%, 60%, 70%, 80%, 90%, 95% or higher, e.g., over a period of seconds, minutes, hours (e.g., 6 hours, 12 hours, or 24 hours), days (e.g., 2 days or 7 days), weeks, or months is encompassed by this term. In certain embodiments, at least 50% of all instances of the L that is cleavable is cleaved after about 10 minutes, about 1 hour, about 6 hours, about 12 hours, about 1 day, about 2 days, about 3 days, about 5 days, or about 7 days of the ultraviolet irradiation, hydrolysis, reduction, oxidation, or contact with the enzyme. In some embodiments, the cleavable linker is cleavable by or is sensitive to an enzyme (e.g., an esterase or a protease), pH (e.g., acidic pH, basic pH), light (e.g., ultraviolet light), a nucleophile, reduction, or oxidation. In some embodiments, the cleavable linker is cleavable by or is sensitive to an enzyme (e.g., an esterase or a protease) or pH (e.g., acidic pH, basic pH). In some embodiments, the cleavable linker is not cleavable by light (e.g., ultraviolet light). In certain embodiments, at least one instance of L is cleavable by ultraviolet irradiation. In certain embodiments, at least one instance of L is cleavable by hydrolysis, reduction, or oxidation. In certain embodiments, at least one instance of L is cleavable by contacting with an enzyme.

The cleavable linker may include an atom or a part of a moiety that is derived in part from the agent (e.g., a therapeutic agent).

In some embodiments, the cleavable linker is cleaved or degraded, e.g., preferentially cleaved or degraded, upon exposure to a first set of conditions relative to a second set of conditions. For example, the cleavable linker can be “preferentially cleaved” or “preferentially degraded” in a first set of conditions relative to a second set of conditions if at least 10%, 20%, 30%, 40%, 50%, 60%, 70%, 80%, 90%, 95% or more of a bond or bonds of the cleavable linker are broken, or the agent is released, in the first set of conditions relative to the second set of conditions.

In some embodiments, the cleavable linker is degraded or hydrolyzed at physiological conditions. In some embodiments, the linker is pH sensitive or cleaved at a certain pH. In some embodiments, the linker is degraded or hydrolyzed through the action of an enzyme (e.g., a protease or esterase). For example, in some embodiments, the cleavable linker is preferentially cleaved in a tissue microenvironment, e.g., a tumor microenvironment, which is referred to herein as a “tissue microenvironment cleavable linker.” In embodiments, the tissue (e.g., tumor) microenvironment cleavable linker is preferentially cleaved or degraded upon exposure to a first desired tissue or tumor microenvironment relative to a second tissue or non-tumor tissue. A tissue (e.g., tumor) microenvironment cleavable linker can be preferentially cleaved if at least 10%, 20%, 30%, 40%, 50%, 60%, 70%, 80%, 90%, 95% or more of a bond or bonds of the linker are broken, or the agent is released, in a desired tissue or tumor microenvironment relative to another tissue or non-tumor tissue. In one embodiment, the tissue (e.g., tumor) microenvironment cleavable linker is preferentially cleaved or degraded if one or more of the bonds of the linker are broken, or the agent is released, at least 2, 5, 10, 20, 30, 40, 50, 60, 70, 80, or 100 times faster upon exposure to a first desired tissue or tumor microenvironment relative to a second tissue or non-tumor tissue. The tissue (e.g., tumor) microenvironment can have a particular set of conditions, e.g., pH, enzymes, that cause the cleavage or degradation of the linker.

In certain embodiments, at least two instances of L are different from each other. In all instances of L are the same.

In one embodiment, the tissue (e.g., tumor) microenvironment cleavable linker is cleavable by an enzyme. In some embodiments, the enzyme comprises an esterase or a protease. Exemplary proteases include MMP-1, MMP-2, MMP-3, MMP-8, MMP-9, MMP-14, plasmin, PSA, PSMA, CATHEPSIN D, CATHEPSIN K, CATHEPSIN S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, or TACE.

In other embodiments, the tissue microenvironment cleavable linker is cleavable at a particular pH. In some embodiments, the tissue microenvironment cleavable linker is cleavable at a pH between about 5.0 and about 7.4, between 5.0 and 7.0, between 5.0 and 6.5, between 5.0 and 5.5, or between 5.9 and 6.2. In one embodiment, the tissue microenvironment cleavable linker is cleavable at a pH between about 6.0 and about 7.0, between about 6.2 and about 6.9, between about 6.5 and about 6.8, or between about 6.5 and about 6.7. In one embodiment, the tissue microenvironment cleavable linker is cleavable at a pH between about 5.5 and about 6.5. e.g., between 5.9 and 6.2. In one embodiment, the tissue microenvironment cleavable linker is cleavable at a hypoxic pH, e.g., a pH about 6.7 to 6.9, e.g., compared to a physiological pH of about 7.4.

In some embodiments, the tissue microenvironment cleavable linker is cleavable is cleaved at a pH of no more than 7.4, no more than 7.0, no more than 6.9, no more than 6.8, no more than 6.7, no more than 6.6, no more than 6.5, no more than 6.4, no more than 6.3, no more than 6.2, no more than 6.1, no more than 6.0, no more than 5.5 or lower.

In one embodiment, the tissue microenvironment cleavable linker is preferentially cleaved or degraded upon exposure to a first pH relative to a second pH. In one embodiment, the tissue microenvironment cleavable linker is cleaved or degraded at least 2, 5, 10, 20, 30, 40, 50, 60, 70, 80, or 100 times faster upon exposure to a first pH relative to a second pH. In other embodiments, the tissue microenvironment cleavable linker shows a greater release or degradation rate at a first acidic pH (e.g., pH=6.7) relative to a second more basic pH (e.g., pH=7.4). In one embodiment, ratio of release or degradation rate of the tissue microenvironment cleavable linker at pH=6.7 relative to pH=7.4 is greater than 1, 1.2, 1.4, 1.6, 1.8, 2, 2.2, 2.4, 2.6, 2.8, 3 or higher. In one embodiment, ratio of release or degradation rate of the tissue microenvironment cleavable linker at pH=6.7 relative to pH=7.4 is greater than 2.

In one embodiment, the tissue microenvironment cleavable linker shows increased pH-sensitivity in a hypoxic microenvironment, e.g., in a tumor, or fibrotic tissue.

In some embodiments, the tissue microenvironment cleavable linker exhibits an increased release rate or increased release yield of the agent at a desired site (e.g., a tumor), e.g., relative to the release rate or release yield at another site. In one embodiment, the tissue microenvironment cleavable linker comprises an electron withdrawing group (e.g., an electron withdrawing group that enhances the cleavage rate or yield, e.g., upon exposure to a first set of conditions relative to a second set of conditions).

In certain embodiments, at least one substituent in at least one instance of L is ═O, halogen (e.g., F), or substituted or unsubstituted, C₁₋₆ alkyl.

In certain embodiments, at least one (e.g., each) instance of W is a single bond. In certain embodiments, at least one (e.g., each) instance of W is —O—. In certain embodiments, at least one (e.g., each) instance of W is —S—. In certain embodiments, at least one (e.g., each) instance of W is —NR^{E}— (e.g., —NH—). In certain embodiments, at least one instance of R^{E} is hydrogen. In certain embodiments, at least one instance of R^{E} is substituted or unsubstituted, C₁₋₆ alkyl. In certain embodiments, at least one instance of R^{E} is Me. In certain embodiments, at least one instance of R^{E} is Et, Pr, Bu, substituted methyl, substituted ethyl, substituted propyl, or substituted butyl. In certain embodiments, at least one instance of R^{E} is a nitrogen protecting group (e.g., Bn, Boc, Cbz, Fmoc, trifluoroacetyl, triphenylmethyl, acetyl, or Ts).

In certain embodiments, at least one (e.g., each) instance of W′ is —O—. In certain embodiments, at least one (e.g., each) instance of W′ is —S—.

In certain embodiments, at least one (e.g., each) instance of R^{a} is hydrogen. In certain embodiments, at least one instance of R^{a} is substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, an oxygen protecting group when attached to an oxygen atom, a sulfur protecting group when attached to a sulfur atom, or a nitrogen protecting group when attached to a nitrogen atom. In certain embodiments, two instances of R^{a} attached to the same nitrogen atom are joined to form substituted or unsubstituted, 3- to 7-membered, monocyclic heterocyclyl, or substituted or unsubstituted, 5- or 6-membered, monocyclic heteroaryl.

In certain embodiments, at least one instance of T is methylene substituted with one or two substituents independently selected from substituted or unsubstituted, C₁₋₆ alkyl and —C(═O)OR (e.g., —C(═O)O(substituted or unsubstituted, C₁₋₆ alkyl), e.g., —C(═O)OMe or —C(═O)OEt). In certain embodiments, at least one instance of T is methylene substituted with one or two unsubstituted C₁₋₃ alkyl (e.g., Me). In certain embodiments, at least one (e.g., each) instance of T is —CH₂—. In certain embodiments, at least one (e.g., each) instance of T is —CH(R^{a})—. In certain embodiments, at least one (e.g., each) instance of T is —CH(CH₃)—. In certain embodiments, at least one instance (e.g., each instance) of T is more stable (e.g., between 30% and 100%, between 1-fold and 10-fold, between 10-fold and 100-fold, between 100-fold and 1,000-fold, between 1,000-fold and 10,000-fold, or between 10,000-fold and 1,000,000-fold, inclusive, more stable) than the moiety —W—C(═O)—W′—. In certain embodiments, being more stable refers to being more chemically stable. In certain embodiments, being more stable refers to being more stable under physiological conditions. In certain embodiments, being more stable refers to being more slowly cleaved by hydrolysis. In certain embodiments, being more stable refers to being more slowly cleaved by light (e.g., ultraviolet light), reduction, or oxidation. In certain embodiments, being more stable refers to being more slowly cleaved by contacting with an enzyme.

In certain embodiments, each instance of R^{B} is hydrogen.

In certain embodiments, each instance of b is independently an integer from 2 to 20, inclusive. In certain embodiments, each instance of b is independently 2, 3, 4, 5, or 6.

In certain embodiments, e is 1. In certain embodiments, e is an integer from 2 to 10, inclusive. In certain embodiments, e is 2 or 3.

In certain embodiments, X is OR^{C}. In certain embodiments, X is N(R^{D})₂. In certain embodiments, R^{C} is hydrogen, substituted or unsubstituted, C₁₋₆ alkyl, an oxygen protecting group, or a leaving group; and at least one instance of R^{D} is hydrogen, substituted or unsubstituted, C₁₋₆ alkyl, or a nitrogen protecting group. In certain embodiments, X is —OR^{C}, wherein R^{C} is an oxygen protecting group or a leaving group. In certain embodiments, X is —OH. In certain embodiments, X is

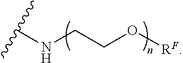

In certain embodiments, X is

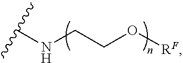

wherein n is an integer from 40 to 100, inclusive; and R^{F} is hydrogen or unsubstituted, C₁₋₆ alkyl.

In certain embodiments, R^{C} or at least one instance of R^{D} is substituted or unsubstituted, C₅₀₋₁₀₀₀ heteroalkyl. In certain embodiments, R^{C} or at least one instance of R^{D} is

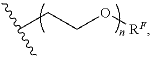

wherein:
n is an integer from 1 to 300, inclusive; and
R^{F} is hydrogen, substituted or unsubstituted, C₁₋₆ alkyl, or an oxygen protecting group.

R^{C} or at least one instance of R^{D} is

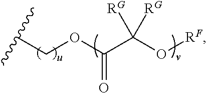

wherein:
u is 1, 2, 3, 4, 5, or 6;
each instance of R^{G} is independently hydrogen, halogen, or substituted or unsubstituted, C₁₋₆alkyl;
v is an integer from 1 to 300, inclusive; and
R^{F} is hydrogen, substituted or unsubstituted, C₁₋₆ alkyl, or an oxygen protecting group.

In certain embodiments,

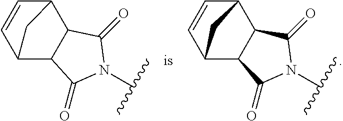 [SMILES: *N1C(=O)C2C3C=CC(C3)C2C1=O ; *N1C(=O)[C@@H]2[C@H](C1=O)[C@H]1C=C[C@@H]2C1]

Exemplary macromonomers may be described by a number of properties, including molecular weight (kDa) and hydrodynamic diameter (nm). In some embodiments, the molecular weight of the macromonomer is between about 1 kDa and about 10 kDa. e.g., between about 2 kDa and about 8 kDa or about 3 kDa and about 6 kDa, e.g., as detected by mass spectrometry. In some embodiments, the molecular weight of the macromonomer is between about 3 kDa and about 6 kDa. In some embodiments, the molecular weight of the macromonomer is about 2 kDa, about 3 kDa, about 4 kDa, about 5 kDa, or about 6 kDa. In some embodiments, the hydrodynamic diameter of the macromonomer is between about 0.5 nm and about 3 nm, e.g., about 1 nm and about 2 nm, e.g., as detected by dynamic light scattering.

In certain embodiments, at least one instance of R¹ is H. In certain embodiments, each instance of R¹ is H.

In certain embodiments, y is 1; [symbol] is a single bond; and R² is hydrogen, halogen, or substituted or unsubstituted, C₁₋₆ alkyl. In certain embodiments, y is 1; [symbol] is a single bond; and R² is hydrogen.

In certain embodiments, y is 1; [symbol] is a single bond; and R³ is hydrogen, halogen, or substituted or unsubstituted, C₁₋₆ alkyl. In certain embodiments, y is 1; [symbol] is a single bond; and R³ is hydrogen.

In certain embodiments, y is 1; [symbol] is a single bond; R² is hydrogen, halogen, or substituted or unsubstituted, C₁₋₆ alkyl; and R³ is hydrogen, halogen, or substituted or unsubstituted, C₁₋₆ alkyl. In certain embodiments, y is 1; [symbol] is a single bond; R² is hydrogen; and R³ is hydrogen.

In certain embodiments, y is 0.

In certain embodiments, y is 0; and R⁴ and R⁶ are joined with their intervening atoms to form substituted or unsubstituted heterocyclyl. In certain embodiments, y is 0; and R⁴ and R⁶ are joined with their intervening atoms to form substituted or unsubstituted pyrrolidinyl, substituted or unsubstituted piperidinyl, substituted or unsubstituted piperazinyl, or substituted or unsubstituted morpholinyl. In certain embodiments, y is 0; and R⁴ and R⁶ are joined with their intervening atoms to form substituted or unsubstituted pyrrolidinyl.

In certain embodiments, y is 1; [symbol] is a double bond; and R³ and R⁴ are cis to each other and are joined with their intervening atoms to form substituted or unsubstituted aryl or substituted or unsubstituted heteroaryl.

In certain embodiments, y is 1; [symbol] is a double bond; and R³ and R⁴ are cis to each other and are joined with their intervening atoms to form substituted or unsubstituted phenyl.

In certain embodiments, y is 1; [symbol] is a double bond; and R³ and R⁴ are cis to each other and are joined with their intervening atoms to form substituted or unsubstituted, 5- or 6-membered, monocyclic heteroaryl (e.g., substituted or unsubstituted pyridyl).

In certain embodiments, [symbol] is a single bond; and R⁵ is hydrogen, halogen, or substituted or unsubstituted, C₁₋₆ alkyl. In certain embodiments, [symbol] is a single bond; and R⁵ is hydrogen.

In certain embodiments, at least one instance of R⁶ is H or substituted or unsubstituted, C₁₋₆ alkyl. In certain embodiments, at least one instance of R⁶ is unsubstituted C₁₋₃ alkyl. In certain embodiments, at least one instance of R⁶ is Me.

### Compounds of Formula (II)

In another aspect, the present disclosure provides compounds of Formula (II):

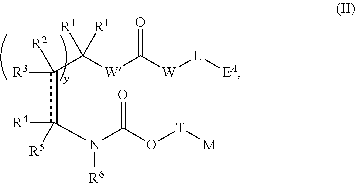

and salts thereof, wherein:
E^{A} is a first reaction handle;
L is substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀ heteroalkylene, substituted or unsubstituted, C₂₋₂₀₀ heteroalkenylene, or substituted or unsubstituted, C₂₋₂₀₀ heteroalkynylene,
optionally one or more carbons in the substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, and substituted or unsubstituted, C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene;
optionally one or more heteroatoms in the substituted or unsubstituted, C₂₋₂₀₀ heteroalkylene, substituted or unsubstituted, C₂₋₂₀₀ heteroalkenylene, and substituted or unsubstituted, C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene;
W is a single bond, —O—, —S—, or —NR^{E}—.

R^{E} is hydrogen, substituted or unsubstituted, C₁₋₆ alkyl, or a nitrogen protecting group;
W′ is —O— or —S—;
each instance of R¹ is independently hydrogen, halogen, or substituted or unsubstituted, C₁₋₆ alkyl;
y is 0 or 1;
when y is 0, [symbol] is a single bond;
when y is 1, [symbol] is a single or double bond;
when [symbol] is a single bond, R² is hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
each instance of R^{a} is independently hydrogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, an oxygen protecting group when attached to an oxygen atom, a sulfur protecting group when attached to a sulfur atom, or a nitrogen protecting group when attached to a nitrogen atom; or two instances of R^{a} attached to the same nitrogen atom are joined to form substituted or unsubstituted, 3- to 7-membered, monocyclic heterocyclyl, or substituted or unsubstituted, 5- or 6-membered, monocyclic heteroaryl;
when [symbol] is a double bond, R² is absent;
R³ is hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
R⁴ is hydrogen, halogen, substituted or unsubstituted, C₁ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
or, when y is 1 and [symbol] is a single bond, R³ and R⁴ are joined with their intervening atoms to form substituted or unsubstituted carbocyclyl or substituted or unsubstituted heterocyclyl;
or, when y is 1 and [symbol] is a double bond, R³ and R⁴ are cis to each other and are joined with their intervening atoms to form substituted or unsubstituted carbocyclyl, substituted or unsubstituted heterocyclyl, substituted or unsubstituted aryl, or substituted or unsubstituted heteroaryl;
or R⁴ and R⁶ are joined with their intervening atoms to form substituted or unsubstituted heterocyclyl;
when [symbol] is a single bond, R is hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
when [symbol] is a double bond, R⁵ is absent;
R⁶ is hydrogen, substituted or unsubstituted, C₁₋₆ alkyl, or a nitrogen protecting group;
T is substituted or unsubstituted methylene; and
M is an ammonium salt or iminium salt of a pharmaceutical agent, wherein the attachment point is the N⁺ of the ammonium salt or iminium salt.

In another aspect, the present disclosure provides compounds of Formula (II):

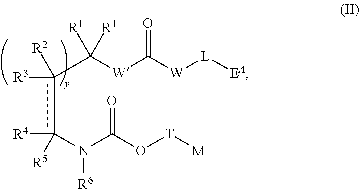

or a salt thereof, wherein:
E^{A} is a first reaction handle;
L is substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀ heteroalkylene, substituted or unsubstituted, C₂₋₂₀₀ heteroalkenylene, or C₂₋₂₀₀ heteroalkynylene,
optionally one or more carbons in the substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, and C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene;
optionally one or more heteroatoms in the substituted or unsubstituted, C₂₋₂₀₀ heteroalkylene, substituted or unsubstituted, C₂₋₂₀₀ heteroalkenylene, and substituted or unsubstituted, C₂₋₂₀ heteroalkynylene are independently replaced with substituted or unsubstituted carbocyclylene, substituted or unsubstituted heterocyclylene, substituted or unsubstituted arylene, or substituted or unsubstituted heteroarylene;
W is a single bond, —O—, —S—, or —NR^{E}—;
R^{E} is hydrogen, substituted or unsubstituted, C₁₋₆ alkyl, or a nitrogen protecting group;
W′ is —O— or —S—;
each instance of R¹ is independently hydrogen, halogen, or substituted or unsubstituted, C₁₋₆ alkyl;
y is 0 or 1;
when y is 0, [symbol] is a single bond;
when y is 1, [symbol] is a single or double bond;
when [symbol] is a single bond, R² is hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR², —NR^{a}C(═O)N(R^{a})₂, —OC(═O)R^{a}, —OC(═O)OR^{a}, or —OC(═O)N(R^{a})₂;
each instance of R^{a} is independently hydrogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, an oxygen protecting group when attached to an oxygen atom, a sulfur protecting group when attached to a sulfur atom, or a nitrogen protecting group when attached to a nitrogen atom; or two instances of R^{a} attached to the same nitrogen atom are joined to form substituted or unsubstituted, 3- to 7-membered, monocyclic heterocyclyl, or substituted or unsubstituted, 5- or 6-membered, monocyclic heteroaryl;
when [symbol] is a double bond, R² is absent;
R³ is hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R)₂, —OC(═O)R^{a}, —OC(═O)OR^{a}, or —OC(═O)N(R^{a})₂;
R⁴ is hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
or, when y is 1 and [symbol] is a single bond, R³ and R⁴ are joined with their intervening atoms to form substituted or unsubstituted carbocyclyl or substituted or unsubstituted heterocyclyl;
or, when y is 1 an [symbol] is a double bond, R³ and R⁴ are cis to each other and are joined with their intervening atoms to form substituted or unsubstituted carbocyclyl, substituted or unsubstituted heterocyclyl, substituted or unsubstituted aryl, or substituted or unsubstituted heteroaryl;
or R⁴ and R⁶ are joined with their intervening atoms to form substituted or unsubstituted heterocyclyl;
when [symbol] is a single bond, R⁵ is hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
when [symbol] is a double bond, R⁵ is absent;
R⁶ is hydrogen, substituted or unsubstituted, C₁₋₆ alkyl, or a nitrogen protecting group;
T is substituted or unsubstituted methylene; and
M is an ammonium salt or iminium salt of a pharmaceutical agent, wherein the attachment point is the N⁺ of the ammonium salt or iminium salt.

Unless otherwise provided, the moieties included in a compound of Formula (II) are as described herein (e.g., in the “Macromonomers” and/or the “Conjugates of Formula (III)” subsections).

The compounds and conjugates may be useful for conjugating with a delivery vehicle (e.g., the moiety D) a pharmaceutical agent that does not contain a conventional reaction handle. In certain embodiments, the conventional reaction handle is —OH, —SH, —NH—, —NH₂, or ═NH. In certain embodiments, the conventional reaction handle is a nucleophile, an electrophile, a leaving group, substituted or unsubstituted, C₂₋₆ alkenyl, substituted or unsubstituted, C₂₋₆ alkynyl, —OH, —SH, —NHR^{a}, —N₃, —C(═O)OH, —C(═NR^{a})OH, —S(═O)OH, —S(═O)₂OH, —C(═O)-(a leaving group), —C(═NR^{a})-(a leaving group), —S(═O)-(a leaving group), or —S(═O)₂-(a leaving group). In certain embodiments, the conventional reaction handle is a nucleophile, an electrophile, a leaving group, —OH, —SH, —NHR^{a}, —N₃, —C(═O)OH, —C(═NR^{a})OH, —S(═O)OH, —S(═O)₂OH, —C(═O)-(a leaving group), —C(═NR^{a})-(a leaving group), —S(═O)-(a leaving group), or —S(═O)₂-(a leaving group). In certain embodiments, the pharmaceutical agent, before conjugation to form the compounds or conjugates, comprises tertiary amino or secondary imine. In certain embodiments, the tertiary amino or secondary imine is the conjugation site when the pharmaceutical agent is conjugated to form the compounds or conjugates.

In certain embodiments, the pharmaceutical agent, after conjugation to form the compounds or conjugates, comprises a quaternary ammonium salt or tertiary iminium salt. Related drug delivery technologies are reported in References (16) to (18).

### Conjugates of Formula (III)

In another aspect, the present disclosure provides conjugates of Formula (III):

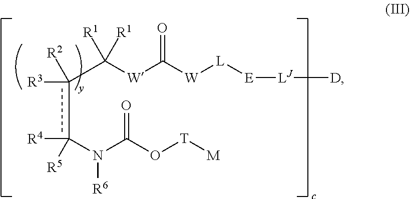

and salts thereof, wherein:
D is a polymeric moiety, dendrimeric moiety, antibody, particle, bead, nanostructure, liposome, micelle, or vesicle;
c is an integer between 1 and 1000, inclusive;
each instance of L^{J} is independently substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, or substituted or unsubstituted, C₂₋₂₀₀ heteroalkynylene, wherein:
   optionally one or more carbons in the substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, and substituted or unsubstituted, C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene; and
   optionally one or more heteroatoms in the substituted or unsubstituted, C₂₋₂₀₀ heteroalkylene, substituted or unsubstituted, C₂₋₂₀₀ heteroalkenylene, and substituted or unsubstituted, C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene;
each instance of E is a moiety formed by reacting E^{A} with E^{B};
each instance of E^{A} is a first reaction handle;
each instance of E^{B} is a second reaction handle, wherein the second reaction handle is able to react with the first reaction handle;
each instance of L is independently substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, or substituted or unsubstituted, C₂₋₂₀₀ heteroalkynylene, wherein:
   optionally one or more carbons in each instance of the substituted or unsubstituted, C₁₋₂₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, and substituted or unsubstituted, C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene; and
   optionally one or more heteroatoms in each instance of the substituted or unsubstituted, C₂₋₂₀₀ heteroalkylene, substituted or unsubstituted, C₂₋₂₀₀ heteroalkenylene, and substituted or unsubstituted, C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene;
each instance of W is independently a single bond, —O—, —S—, or —NR^{E}—;
each instance of R^{E} is independently hydrogen, substituted or unsubstituted, C₁₋₆ alkyl, or a nitrogen protecting group;
each instance of W′ is independently —O— or —S—;
each instance of R¹ is independently hydrogen, halogen, or substituted or unsubstituted, C₁₋₆ alkyl;
each instance of y is independently 0 or 1;
when y is 0, [symbol] is a single bond;
when y is 1, [symbol] is a single or double bond;
when [symbol] is a single bond, R² is hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
each instance of R^{a} is independently hydrogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, an oxygen protecting group when attached to an oxygen atom, a sulfur protecting group when attached to a sulfur atom, or a nitrogen protecting group when attached to a nitrogen atom; or two instances of R^{a} attached to the same nitrogen atom are joined to form substituted or unsubstituted, 3- to 7-membered, monocyclic heterocyclyl, or substituted or unsubstituted, 5- or 6-membered, monocyclic heteroaryl;
when [symbol] is a double bond, R² is absent;
each instance of R³ is independently hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
each instance of R⁴ is independently hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR², —NR^{a}C(═O)N(R^{a})₂, —OC(═O)R^{a}, —OC(═O)OR^{a}, or —OC(═O)N(R^{a})₂;
or, when y is 1 and [symbol] is a single bond, R³ and R⁴ are joined with their intervening atoms to form substituted or unsubstituted carbocyclyl or substituted or unsubstituted heterocyclyl;
or, when y is 1 and [symbol] is a double bond, R³ and R⁴ are cis to each other and are joined with their intervening atoms to form substituted or unsubstituted carbocyclyl, substituted or unsubstituted heterocyclyl, substituted or unsubstituted aryl, or substituted or unsubstituted heteroaryl;
or R⁴ and R⁶ are joined with their intervening atoms to form substituted or unsubstituted heterocyclyl;
when [symbol] is a single bond, R⁵ is hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}. —NR^{a}C(═O)OR^{a}, —NRC(═O)N(R)₂, —OC(═O)R^{a}, —OC(═O)OR^{a}, or —OC(═O)N(R^{a})₂;
when [symbol] is a double bond, R⁵ is absent;
each instance of R⁶ is independently hydrogen, substituted or unsubstituted, C₁₋₆ alkyl, or a nitrogen protecting group;
each instance of T is independently substituted or unsubstituted methylene; and
each instance of M is independently an ammonium salt or iminium salt of a pharmaceutical agent, wherein the attachment point is the N⁺ of the ammonium salt or iminium salt.

In another aspect, the present disclosure provides conjugates of Formula (III):

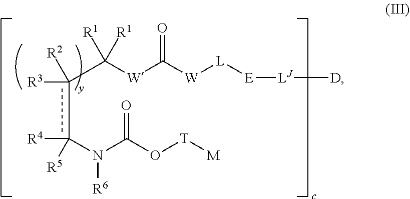

and salts thereof, wherein:
D is a polymeric moiety, dendrimeric moiety, antibody, particle, bead, nanostructure, liposome, micelle, or vesicle;
c is an integer between 1 and 1000, inclusive;
each instance of L^{J} is independently substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀ heteroalkenylene, or C₂₋₂₀₀ heteroalkynylene, wherein:
   optionally one or more carbons in the substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, and C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene; and
   optionally one or more heteroatoms in the substituted or unsubstituted, C₂₋₂₀₀ heteroalkylene, substituted or unsubstituted, C₂₋₂₀₀ heteroalkenylene, and substituted or unsubstituted, C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene;
each instance of E is a moiety formed by reacting E^{A} with E^{B};
each instance of E^{A} is a first reaction handle;
each instance of E^{B} is a second reaction handle, wherein the second reaction handle is able to react with the first reaction handle;
each instance of L is independently substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, or C₂₋₂₀₀ heteroalkynylene, wherein:
   optionally one or more carbons in each instance of the substituted or unsubstituted, C₁₋₂₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, and C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene; and
   optionally one or more heteroatoms in each instance of the substituted or unsubstituted, C₂₋₂₀ heteroalkylene, substituted or unsubstituted, C₂₋₂₀₀ heteroalkenylene, and substituted or unsubstituted, C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene;
each instance of W is independently a single bond, —O—, —S—, or —NR^{E}—;
each instance of R^{E} is independently hydrogen, substituted or unsubstituted, C₁₋₆ alkyl, or a nitrogen protecting group;
each instance of W′ is independently —O— or —S—;
each instance of R¹ is independently hydrogen, halogen, or substituted or unsubstituted, C₁₋₆ alkyl;
each instance of y is independently 0 or 1;
when y is 0, [symbol] is a single bond;
when y is 1, [symbol] is a single or double bond;
when [symbol] is a single bond, R² is hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR²C(═O)OR², —NR^{a}C(═O)N(R^{a})₂, —OC(═O)R^{a}, —OC(═O)OR^{a}, or —OC(═O)N(R^{a})₂;
each instance of R^{a} is independently hydrogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, an oxygen protecting group when attached to an oxygen atom, a sulfur protecting group when attached to a sulfur atom, or a nitrogen protecting group when attached to a nitrogen atom; or two instances of R^{a} attached to the same nitrogen atom are joined to form substituted or unsubstituted, 3- to 7-membered, monocyclic heterocyclyl, or substituted or unsubstituted, 5- or 6-membered, monocyclic heteroaryl;
when [symbol] is a double bond, R² is absent;
each instance of R³ is independently hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{aa}, —CN, —SCN, —C(═NR^{a})R^{a}, —C(═NR^{a})OR—, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
each instance of R⁴ is independently hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
or, when y is 1 and [symbol] is a single bond, R³ and R⁴ are joined with their intervening atoms to form substituted or unsubstituted carbocyclyl or substituted or unsubstituted heterocyclyl;
or, when y is 1 and [symbol] is a double bond, R³ and R⁴ are cis to each other and are joined with their intervening atoms to form substituted or unsubstituted carbocyclyl, substituted or unsubstituted heterocyclyl, substituted or unsubstituted aryl, or substituted or unsubstituted heteroaryl;
or R⁴ and R⁶ are joined with their intervening atoms to form substituted or unsubstituted heterocyclyl;
when [symbol] is a single bond, R⁵ is hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
when [symbol] is a double bond, R⁵ is absent;
each instance of R⁶ is independently hydrogen, substituted or unsubstituted, C₁₋₆ alkyl, or a nitrogen protecting group;
each instance of T is independently substituted or unsubstituted methylene; and
each instance of M is independently an ammonium salt or iminium salt of a pharmaceutical agent, wherein the attachment point is the N⁺ of the ammonium salt or iminium salt.

Unless otherwise provided, the moieties included in a conjugate of Formula (III) are as described herein (e.g., in the “Macromonomers” subsection).

The compounds may be conjugated with D, which may be a delivery vehicle, to form the conjugates. The conjugates may be useful for. e.g., delivering the pharmaceutical agent.

In certain embodiments, D is a brush polymeric moiety or brush-arm star polymeric moiety. In certain embodiments, D is a nanoparticle or microparticle. In certain embodiments, D is an antibody.

In certain embodiments, at least one instance of E is a moiety formed by reacting two click-chemistry handles (e.g., two orthogonal click-chemistry handles). In certain embodiments, at least one instance of the click-chemistry handle comprises C≡C or C═C.

In certain embodiments, at least one instance of the click-chemistry handle comprises C≡CH, C═CH, CH═CH, C═CH₂, or CH═CH₂. In certain embodiments, at least one instance of the click-chemistry handle is —C≡CH, substituted or unsubstituted cyclooctynyl optionally fused independently with one or more instances of substituted or unsubstituted phenyl, substituted or unsubstituted cyclopropenyl, substituted or unsubstituted cyclobutenyl, substituted or unsubstituted trans-cyclooctenyl optionally fused independently with one or more instances of substituted or unsubstituted phenyl, or substituted or unsubstituted

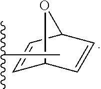

In certain embodiments, each instance of the click-chemistry handle is —C≡CH. In certain embodiments, at least one instance of the click-chemistry handle is —N₃. In certain embodiments, each instance of the click-chemistry handle is —N₃. In certain embodiments, at least one instance of E is a single bond, —O—, —S—, —NR^{a}—, —C(═O)O—, —C(═NR^{a})O—, —S(═O)O—, —S(═O)₂O—, —C(═O)NR^{a}—, —C(═NR^{a})NR^{a}—, —S(═O)NR^{a}—, —S(═O)₂NR^{a}—, —OC(═O)—, —OC(═NR^{a})—, —OS(═O)—, —OS(═O)₂—, —NR^{a}C(═O)—, —NR^{a}C(═NR^{a})—, —NR^{a}S(═O)—, —NR^{a}S(═O)₂—, —OC(═O)O—, —OC(═NR^{a})O—, —OS(═O)O—, —OS(═O)₂O—, —NR^{a}C(═O)O—, —NR^{a}C(═NR^{a})O—, —NR^{a}S(═O)O—, —NR^{a}S(═O)₂O—, —OC(═O)NR^{a}—, —OC(═NR^{a})NR^{a}—, —OS(═O)NR^{a}—, —OS(═O)₂NR^{a}—, —NR^{a}C(═O)NR^{a}—, —NR^{a}C(═NR^{a})NR^{a}—, —NR^{a}S(═O)NR^{a}—, —NR^{a}S(═O)₂NR^{a}—, —C(═O)—, —C(═NR^{a})—, —S(═O)—, or —S(═O)₂—.

In certain embodiments, at least one instance of E is

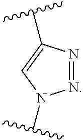 [SMILES: *c1cn(*)nn1]

In certain embodiments, at least one instance of

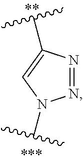 [SMILES: *c1cn(*)nn1]

herein the nitrogen atom labeled with “*” is closer to the attachment point labeled with “**” than the attachment point labeled with “***”. In certain embodiments, each instance of E is

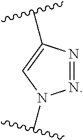 [SMILES: *c1cn(*)nn1]

In certain embodiments, each instance of E is

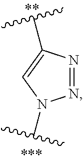 [SMILES: *c1cn(*)nn1]

wherein the nitrogen atom labeled with “*” is closer to the attachment point labeled with “**” than the attachment point labeled with “***”.

In certain embodiments, at least one instance of E^{A} is a polymerization handle. In certain embodiments, at least one instance of E^{A} is an addition polymerization handle or condensation polymerization handle. In certain embodiments, at least one instance of E^{A} is a metathesis polymerization handle. In certain embodiments, at least one instance of E^{A} is substituted or unsubstituted, C₂₋₆ alkenyl or substituted or unsubstituted, C₂₋₆ alkynyl. In certain embodiments, at least one instance of E^{A} is —OH, —NH₂, —C(═O)OH, or —C(═O)H. In certain embodiments. In certain embodiments, at least one instance of E^{A} is a nucleophile, an electrophile, a leaving group, substituted or unsubstituted, C₂₋₆ alkenyl, substituted or unsubstituted, C₂₋₆ alkynyl, —OH, —SH, —NHR^{a}, —N₃, —C(═O)OH, —C(═O)N(R^{a})₂, —C(═NR^{a})OH, —S(═O)OH, —S(═O)₂OH, —C(═O)-(a leaving group), —C(═NR^{a})-(a leaving group), —S(═O)-(a leaving group), or —S(═O)₂-(a leaving group). In certain embodiments, at least one instance of E^{A} is a click-chemistry handle. In certain embodiments, each instance of E^{A} is a click-chemistry handle. In certain embodiments, at least one instance of E^{A} is —N₃. In certain embodiments, each instance of E^{A} is —N₃.

In certain embodiments, at least one instance of E^{B} is a click-chemistry handle. In certain embodiments, each instance of E^{B} is a click-chemistry handle. In certain embodiments, at least one instance of E^{B} is —C≡CH. In certain embodiments, E^{B} is —C≡CH.

In certain embodiments, each instance of E^{A} and each instance of E^{B} are orthogonal click-chemistry handles. In certain embodiments, each instance of E^{A} is —N₃, and each instance of E^{B} is —C≡CH.

In certain embodiments, at least one instance of E^{B} is a nucleophile, an electrophile, a leaving group, substituted or unsubstituted, C₂₋₆ alkenyl, substituted or unsubstituted, C₂₋₆ alkynyl, —OH, —SH, —NHR^{a}, —N₃, —C(═O)OH, —C(═O)N(R′)₂, —C(═NR^{a})OH, —S(═O)OH, —S(═O)₂OH, —C(═O)-(a leaving group), —C(═NR^{a})-(a leaving group), —S(═O)-(a leaving group), or —S(═O)₂-(a leaving group).

In certain embodiments, at least one instance of L^{J} is substituted or unsubstituted, C₁₋₁₂ alkylene, or substituted or unsubstituted, C₂₋₁₂ heteroalkylene. In certain embodiments, at least one instance of L^{J} is substituted (e.g., substituted with one or more of: halogen, substituted or unsubstituted, C₁₋₆ alkyl, —OR^{a}, and/or oxo) or unsubstituted, C₁₋₁₂ alkylene. In certain embodiments, at least one instance of L^{J} is or substituted (e.g., substituted with one or more of: halogen, substituted or unsubstituted, C₁₋₆ alkyl. —OR^{a}, and/or oxo) or unsubstituted, C₂₋₁₂ heteroalkylene. In certain embodiments, at least one instance of L^{J} is unsubstituted C₁₋₆ alkylene. In certain embodiments, each instance of L^{J} is unsubstituted C₁₋₆ alkylene. In certain embodiments, at least one instance (e.g., each instance) of L¹ is —CH₂—.

In certain embodiments, c is an integer between 1 and 100 (e.g., between 1 and 10, between 11 and 30, between 31 and 100), inclusive. In certain embodiments, c is an integer between 100 and 300, inclusive. In certain embodiments, c is an integer between 300 and 1000, inclusive.

In certain embodiments, a conjugate includes salts thereof.

### Methods of Preparing the Macromonomers, Compounds, and Conjugates

In another aspect, the present disclosure provides methods of preparing the macromonomers, and salts thereof.

In certain embodiments, a method of preparing a macromonomer, or a salt thereof, comprises coupling a compound of the formula:

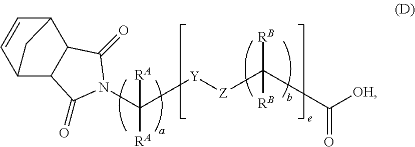

or a salt thereof, with a compound of the formula: HOR^{C} or HN(R^{D})₂, or a salt thereof. In certain embodiments, the step of coupling is performed in the presence of a reagent for coupling a carboxylic acid with an alcohol or amine.

M can be conjugated to the macromonomer using any suitable conjugation technique. For instance, EDC-NHS chemistry (1-ethyl-3-(3-dimethylaminopropyl) carbodiimide hydrochloride and N-hydroxysuccinimide), or a reaction involving a maleimide or a carboxylic acid, which can be conjugated to one end of a thiol, an amine, or a similarly functionalized polyether. The conjugation can be performed in an organic solvent, such as, but not limited to, methylene chloride, acetonitrile, chloroform, dimethylformamide, tetrahydrofuran, acetone, or the like. Specific reaction conditions can be determined by those of ordinary skill in the art using no more than routine experimentation.

In another set of embodiments, a conjugation reaction may be performed by reacting the agent that includes a hydroxyl, thiol, or amino group with a polymer comprising a carboxylic acid functional group. Such a reaction may occur as a single-step reaction, i.e., the conjugation is performed with or without using intermediates such as N-hydroxysuccinimide or a maleimide. The conjugation reaction between the amine-containing, thiol-containing, or hydroxyl-containing moiety and the carboxylic acid-terminated polymer may be achieved in one embodiment, by adding the amine-containing, thoil-containing, or hydroxyl-containing moiety, solubilized in an organic solvent such as, but not limited to, dichloromethane, acetonitrile, chloroform, tetrahydrofuran, acetone, formamide, dimethylformamide, pyridines, dioxane, or dimethysulfoxide, to a solution containing the carboxylic acid-terminated polymer. The carboxylic acid-terminated polymer may be contained within an organic solvent such as, but not limited to, dichloromethane, acetonitrile, chloroform, dimethylformamide, tetrahydrofuran, or acetone. Reaction between the amine-containing moiety and the carboxylic acid-terminated polymer may occur spontaneously in some cases.

Unconjugated macromonomers may be washed away after such reactions, and the polymer may be precipitated in solvents such as, for instance, ethyl ether, hexane, methanol, or ethanol.

In certain embodiments, a reagent for coupling a carboxylic acid with an alcohol or amine is N-(3-dimethylaminopropyl)-N′-ethylcarbodiimide (EDC), dicyclohexylcarbodiimide (DCC), 1-ethyl-3-(3-dimethylaminopropyl)carbodiimide hydrochloride (WSC/HCl), diphenylphosphorylazide (DPPA), carbonyldiimidazole (CDI), diethylcyanophosphonate (DEPC), benzotriazole-1-yloxy-trispyrrolidinophosphonium (DIPCI), benzotriazole-1-yloxy-trispyrrolidinophosphonium hexafluorophosphate (PyBOP), 1-hydroxybenzotriazole (HOBt), hydroxysuccinimide (HOSu), dimethylaminopyridine (DMAP), 1-hydroxy-7-azabenzotriazole (HOAt), hydroxyphthalimide (HOPht), pentafluorophenol (Pfp-OH), 2-(1H-benzotriazole-1-yl)-1,1,3,3-tetramethyluronium hexafluorophosphate (HBTU), 0-(7-azabenzotriazole-1-yl)-1,1,3,3-tetramethyluronium hexafluorophosphonate (HATU), O-benzotriazole-1-yl-1,1,3,3-tetramethyluronium tetrafluoroborate (TBTU), or 3,4-dihydro-3-hydrodi-4-oxa-1,2,3-benzotriazine (Dhbt), or a salt thereof; or a combination (e.g., a combination of two) thereof. In certain embodiments, the reagent for coupling a carboxylic acid with an alcohol or amine is DCC. In certain embodiments, the reagent for coupling a carboxylic acid with an alcohol or amine is EDC, or a salt thereof.

The reagent for coupling a carboxylic acid with an alcohol or amine is used in an amount of about 1 to 20 equivalents of the compound of Formula (D). In certain embodiments, the reagent for coupling a carboxylic acid with an alcohol or amine is used in an amount of about 1 to 10 equivalents. In certain embodiments, the activator is used in an amount of about 1 to 5 equivalents.

Examples of useful solvents in the coupling reaction are DMSO, DMF, and methylene chloride. Additional exemplary solvents include acetonitrile, chloroform, tetrahydrofuran, and acetone.

The coupling reaction can be conducted at 0 to 50° C. In certain embodiments, the coupling reaction is conducted at room temperature for about 10 min to about 30 hours. In certain embodiments, the coupling reaction is conducted for about 15 minutes to about 24 hours.

In another aspect, the present disclosure provides methods of preparing the compounds, and salts thereof. In certain embodiments, the present disclosure provides methods of preparing the compounds of Formula (II) comprising reacting a compound of the formula:

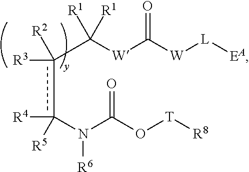

or a salt thereof, with the pharmaceutical agent in the presence of a halide, wherein R⁸ is halogen.

In certain embodiments, the halide is an alkali metal iodide (e.g., NaI).

In certain embodiments, R⁸ is C₁.

In certain embodiments, the step of reacting is performed under a temperature between 2° and 40° C., inclusive. In certain embodiments, the step of reacting is performed under a temperature between 4° and 80° C. inclusive. In certain embodiments, the step of reacting is performed under a temperature between 5° and 70° C., inclusive. In certain embodiments, the step of reacting is performed under a temperature of about 60° C.

In another aspect, the present disclosure provides methods of preparing a compound of Formula (II′):

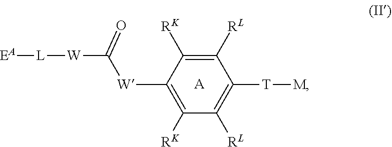

or a salt thereof, comprising reacting a compound of the formula:

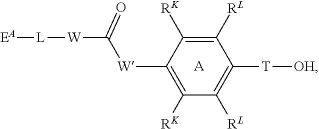

or a salt thereof, with a pharmaceutical agent in the presence of a strong electrophile and a base at a temperature not higher than 20° C., wherein:
E^{A} is a first reaction handle;
L is substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, or substituted or unsubstituted, C₂₋₂₀₀ heteroalkynylene,
optionally one or more carbons in the substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, and substituted or unsubstituted, C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene;
optionally one or more heteroatoms in the substituted or unsubstituted, C₂₋₂₀₀ heteroalkylene, substituted or unsubstituted, C₂₋₂₀₀ heteroalkenylene, and substituted or unsubstituted, C₂₋₂₀ heteroalkynylene are independently replaced with substituted or unsubstituted carbocyclylene, substituted or unsubstituted heterocyclylene, substituted or unsubstituted arylene, or substituted or unsubstituted heteroarylene;
W is a single bond, —O—, —S—, or —NR^{E}—;
R^{E} is hydrogen, substituted or unsubstituted, C₁₋₆ alkyl, or a nitrogen protecting group;
W′ is —O—, —S—, or —NR^{J}—;
R^{J} is hydrogen, substituted or unsubstituted, C₁₋₆ alkyl, or a nitrogen protecting group;
each instance of R^{K} and R^{L} is independently hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
each instance of R^{a} is independently hydrogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, an oxygen protecting group when attached to an oxygen atom, a sulfur protecting group when attached to a sulfur atom, or a nitrogen protecting group when attached to a nitrogen atom; or two instances of R^{a} attached to the same nitrogen atom are joined to form substituted or unsubstituted, 3- to 7-membered, monocyclic heterocyclyl, or substituted or unsubstituted, 5- or 6-membered, monocyclic heteroaryl;
T is substituted or unsubstituted methylene; and
M is an ammonium salt or iminium salt of a pharmaceutical agent, wherein the attachment point is the N⁺ of the ammonium salt or iminium salt.

In certain embodiments, the present disclosure provides methods of preparing a compound of Formula (II′):

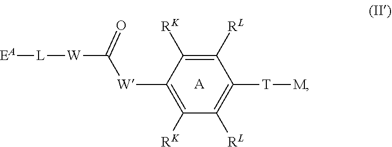

or a salt thereof, comprising reacting a compound of the formula:

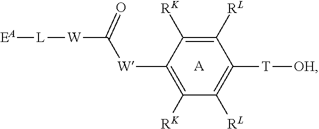

or a salt thereof, with a pharmaceutical agent in the presence of a strong electrophile and a base at a temperature not higher than 20° C., wherein:
E^{A} is a first reaction handle;
L is substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀ heteroalkylene, substituted or unsubstituted, C₂₋₂₀₀ heteroalkenylene, or C₂₋₂₀₀ heteroalkynylene,
optionally one or more carbons in the substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, and C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene;
optionally one or more heteroatoms in the substituted or unsubstituted, C₂₋₂₀₀ heteroalkylene, substituted or unsubstituted, C₂₋₂₀₀ heteroalkenylene, and substituted or unsubstituted, C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene;
W is a single bond, —O—, —S—, or —NR^{E}—;
R^{F} is hydrogen, substituted or unsubstituted, C₁₋₆ alkyl, or a nitrogen protecting group;
W′ is —O—, —S—, or —NR^{J}—;
R¹ is hydrogen, substituted or unsubstituted, C₁₋₆ alkyl, or a nitrogen protecting group;
each instance of R^{K} and R^{L} is independently hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R′)₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R)₂;
each instance of R^{a} is independently hydrogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, an oxygen protecting group when attached to an oxygen atom, a sulfur protecting group when attached to a sulfur atom, or a nitrogen protecting group when attached to a nitrogen atom; or two instances of R^{a} attached to the same nitrogen atom are joined to form substituted or unsubstituted, 3- to 7-membered, monocyclic heterocyclyl, or substituted or unsubstituted, 5- or 6-membered, monocyclic heteroaryl;
T is substituted or unsubstituted methylene; and
M is an ammonium salt or iminium salt of a pharmaceutical agent, wherein the attachment point is the N⁺ of the ammonium salt or iminium salt.

In certain embodiments, the strong electrophile is a stronger electrophile than iodo. In certain embodiments, the strong electrophile is a sulfonic anhydride or a sulfonyl halide. In certain embodiments, the strong electrophile is a sulfonic anhydride (e.g., p-toluenesulfonic anhydride, methanesulfonic anhydride, p-bromobenzenesulfonic anhydride, O[S(═O)₂(CF₂)₃CF₃]₂, or triflate anhydride. In certain embodiments, the strong electrophile is a triflate. In certain embodiments, the strong electrophile is triflate anhydride. In certain embodiments, the strong electrophile is a sulfonyl halide (e.g., p-toluenesulfonyl halide, methanesulfonyl halide, p-bromobenzenesulfonyl halide, X[S(═O)₂(CF₂)₃CF₃] wherein X is halogen, or triflic halide).

In certain embodiments, the base is a tertiary non-aromatic amine or an aromatic amine that does not comprise —NH—. In certain embodiments, the base is tertiary non-aromatic amine. In certain embodiments, the base is tertiary non-aromatic amine that does not comprise —NH— or —NH₂. In certain embodiments, the base is aromatic amine that does not comprise —NH—. In certain embodiments, the base is aromatic amine that does not comprise —NH— or —NH₂. In certain embodiments, the base is trialkylamine (e.g., N,N-diisopropylethylamine).

In certain embodiments, the temperature is between 0 and 20° C., inclusive. In certain embodiments, the temperature is between −30 and 0° C., inclusive. In certain embodiments, the temperature is between −60 and −30° C., inclusive. In certain embodiments, the temperature is between −100 and −60° C., inclusive. In certain embodiments, the temperature is between −90 and −70° C., inclusive. In certain embodiments, the temperature is about −78° C.

The methods of preparing a compound of Formula (II′) may be advantageous over reported methods that involve high temperatures (e.g., about 60° C.) and/or iodide (e.g., iodide electrophiles (e.g., NaI)). The reported methods may cause the pharmaceutical agent to degrade. By using a strong electrophile (e.g., triflate) as the leaving group, which is more electrophilic than iodide, the methods of preparing a compound of Formula (II′) may be able to reduce the temperature necessary to functionalize normally unstable pharmaceutical agents (e.g., with azides using the ammonium based linker chemistry). At about −78° C. for example, JQ1, AZD5153, and vincristine were conjugated by the methods of preparing a compound of Formula (II′) without significant degradation. An example is shown in the scheme below.

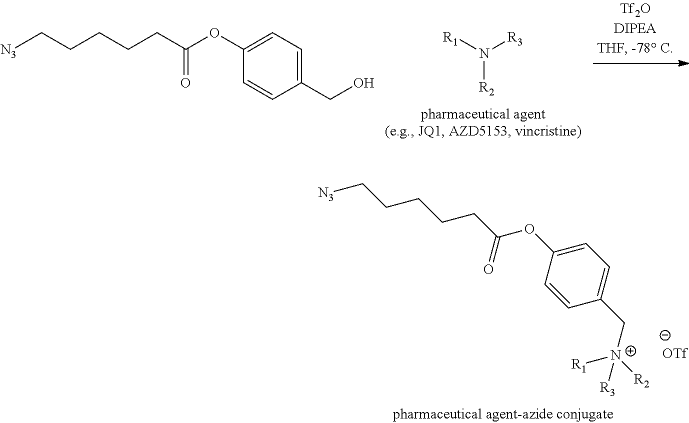

### Brush Prodrug (Polymers) and Methods of Preparing the Brush Prodrugs

In another aspect, the present disclosure provides Brush prodrugs (polymers). In certain embodiments, the Brush prodrugs are prepared by polymerizing a macromonomer, or a salt thereof, in the presence of a metathesis catalyst. In certain embodiments, at least one instance of M of the first macromonomer is different from at least one instance of M of the second macromonomer. In certain embodiments, the metathesis catalyst is a transition metal metathesis catalyst (e.g., ruthenium metathesis catalyst) or Grubbs catalyst. In certain embodiments, the metathesis catalyst is of the formula:

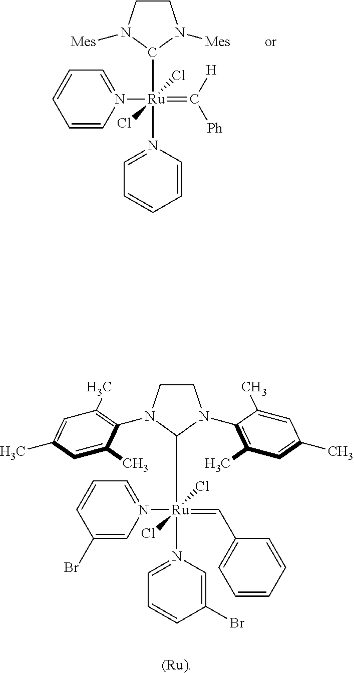

The methods for preparing the Brush prodrugs described herein may involve ring-opening metathesis polymerization (ROMP) (Liu et al. J. Am. Chem. Soc. 2012, 134, 16337; Liu, J.; Gao, A. X.; Johnson, J. A. J Vis Exp 2013, e50874). In certain embodiments, the Brush prodrugs described herein are prepared by polymerization of norbornene-terminated macromonomers followed by in situ crosslinking with bis-norbornene crosslinkers. The preparation methods described herein are versatile and have little limitations, e.g., in terms of the different agents that can be built into the Brush prodrugs. In certain embodiments, an agent that can be built into the Brush prodrugs includes addressable functional groups that are compatible with ROMP.

In certain embodiments, the metathesis catalyst (e.g., ROMP catalyst) is a tungsten (W), molybdenum (Mo), or ruthenium (Ru) catalyst. In certain embodiments, the ROMP catalyst is a ruthenium catalyst. ROMP catalysts useful in the synthetic methods described herein include catalysts as depicted below, and as described in Grubbs et al., Acc. Chem. Res. 1995, 28, 446-452; U.S. Pat. No. 5,811,515; Schrock et al., Organometallics (1982) 1 1645; Gallivan et al., Tetrahedron Letters (2005) 46:2577-2580; Furstner et al., J. Am. Chem. Soc. (1999) 121:9453; and Chem. Eur. J. (2001) 7:5299; the entire contents of each of which are incorporated herein by reference.

In certain embodiments, the ROMP catalyst is a Grubbs catalyst. In certain embodiments, the Grubbs catalyst is selected from the group consisting of:

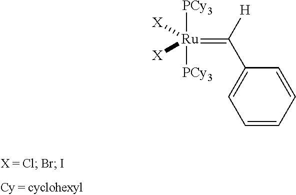

Benzylidenebis-(tricyclohexylphosphine)-dichlororuthenium (X=Cl);
Benzylidenebis-(tricyclohexylphosphine)-dibromoruthenium (X=Br);
Benzylidenebis-(tricyclohexylphosphine)-diiodoruthenium (X=I);

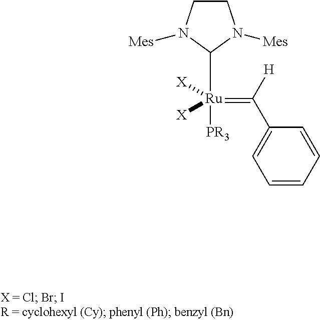

1,3-(Bis(mesityl)-2-imidazolidinylidene)dichloro-(phenylmethylene) (tricyclohexyl-phosphine)ruthenium (X=Cl; R=cyclohexyl);
1,3-(Bis(mesityl)-2-imidazolidinylidene)dibromo-(phenylmethylene) (tricyclohexyl-phosphine)ruthenium (X=Br; R=cyclohexyl);
1,3-(Bis(mesityl)-2-imidazolidinylidene)diiodo-(phenylmethylene) (tricyclohexyl-phosphine)ruthenium (X=I; R=cyclohexyl);
1,3-(Bis(mesityl)-2-imidazolidinylidene)dichloro-(phenylmethylene) (triphenylphosphine)ruthenium (X=Cl; R=phenyl);
1,3-(Bis(mesityl)-2-imidazolidinylidene)dichloro-(phenylmethylene) (tribenzylphosphine)ruthenium (X=Cl; R=benzyl);

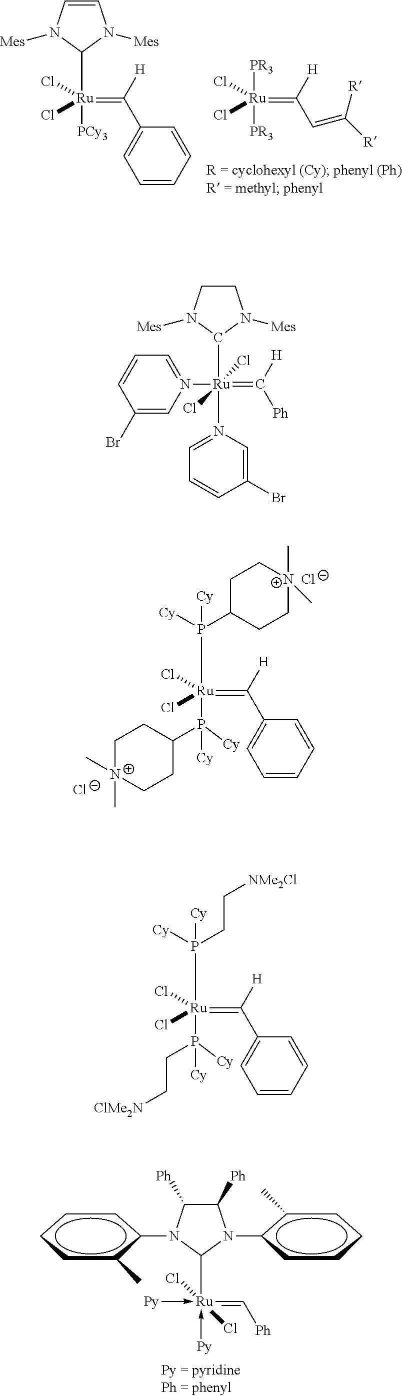

In certain embodiments, the ROMP catalyst is a Grubbs-Hoveyda catalyst. In certain embodiments, the Grubbs-Hoveyda catalyst is selected from the group consisting of:

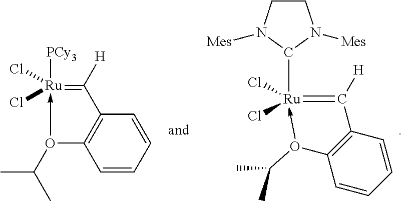

In certain embodiments, the ROMP catalyst is selected from the group consisting of:

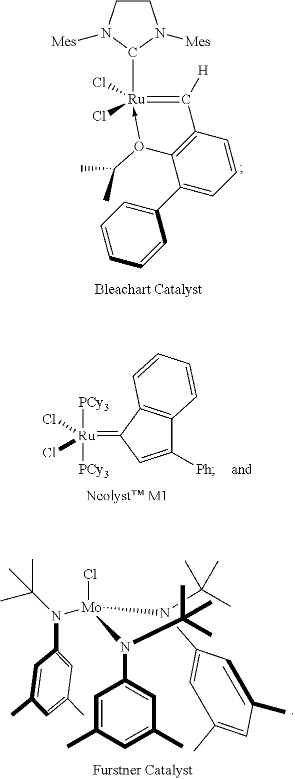

In certain embodiments, the ROMP catalyst is of the formula:

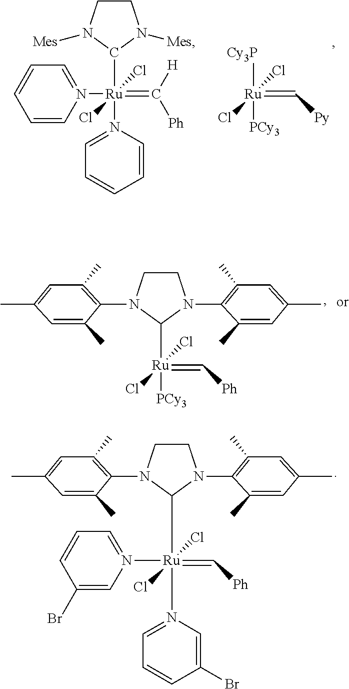

The ROMP can be conducted in one or more aprotic solvents. The term “aprotic solvent” means a non-nucleophilic solvent having a boiling point range above ambient temperature, preferably from about 25° C. to about 190° C. at atmospheric pressure. In certain embodiments, the aprotic solvent has a boiling point from about 80° C. to about 160° C. at atmospheric pressure. In certain embodiments, the aprotic solvent has a boiling point from about 80° C. to about 150° C. at atmospheric pressure. Examples of such solvents are methylene chloride, acetonitrile, toluene, DMF, diglyme, THF, and DMSO.

The ROMP can be quenched with a vinyl ether of the formula

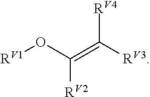

Each of R^{V1}, R^{V2}, R^{V3}, and R^{V4} is independently optionally substituted alkyl, optionally substituted alkenyl, optionally substituted alkynyl, optionally substituted carbocyclyl, optionally substituted phenyl, optionally substituted heterocyclyl, or optionally substituted heteroaryl. In certain embodiments, R^{V1} is optionally substituted alkyl, and R^{V2}, R^{V3}, and R^{V4} are hydrogen. In certain embodiments, R^{V1} is unsubstituted alkyl, and R^{V2}, R^{V}3, and R^{V4} are hydrogen. In certain embodiments, R^{V1} is substituted alkyl, and R^{V2}, R^{V3}, and R^{V4} are hydrogen. In certain embodiments, R^{V1} is methyl, and R^{V2}, R^{V3}, and R^{V4} are hydrogen. In certain embodiments, R^{V1} is ethyl, and R^{V2}, R^{V3}, and R^{V4} are hydrogen. In certain embodiments, R^{V1} is propyl, and R^{V2}, R^{V3}, and R^{V4} are hydrogen. In certain embodiments, R^{V1} is optionally substituted alkenyl, and R^{V2}, R^{V3}, and R^{V4} are hydrogen. In certain embodiments, R^{V1} is unsubstituted alkenyl, and R^{V2}, R^{V3}, and R^{V4} are hydrogen. In certain embodiments, R^{V1} is vinyl, and R^{V2}, R^{V3}, and R^{V4} are hydrogen. In certain embodiments, at least one of R^{V1}, R^{V2}, R^{V3}, and R^{V4} is conjugated with a diagnostic agent as defined above. In certain embodiments, the ROMP is quenched by ethyl vinyl ether. Excess ethyl vinyl ether can be removed from the Brush prodrugs by vacuum.

In another aspect, the present disclosure provides methods of preparing the Brush prodrug prodrugs.

The Brush prodrugs may be described by a number of properties, including average molecular weight (kDa), average hydrodynamic diameter (nm), and polydispersity.

The term “average molecular weight” may encompass the number average molecular weight (Mn), weight average molecular weight (Mw), higher average molecular weight (Mz or Mz+1), GPC/SEC-determined average molecular weight (Mp), and viscosity average molecular weight (Mv). In certain embodiments, the average molecular weight is Mw. In certain embodiments, the Mn is determined with gel permeation chromatography, viscometry via the (Mark-Houwink equation), colligative methods (such as vapor pressure osmometry), end-group determination, or proton NMR. In certain embodiments, the Mw is determined with static light scattering, small angle neutron scattering, X-ray scattering, and sedimentation velocity. In some embodiments, the average molecular weight of the Brush prodrug is between about 10 kDa and about 100 kDa, e.g., between about 15 kDa and about 85 kDa, about 20 kDa and about 60 kDa. or about 30 kDa and about 50 kDa, e.g., as determined by gel permeation chromatography. In one embodiment, the average molecular weight of the Brush prodrug is between about 20 kDa and about 60 kDa. In one embodiment, the average molecular weight of the Brush prodrug is between about 30 kDa and about 50 kDa.

In some embodiments, the average molecular weight of the Brush prodrug is less than about 100 kDa (e.g., less than about 95 kDa, about 90 kDa, about 85 kDa, about 80 kDa, about 75 kDa, about 70 kDa, about 65 kDa, about 60 kDa, about 55 kDa, or about 50 kDa), e.g., as determined by gel permeation chromatography. In some embodiments, the average molecular weight of the Brush prodrug is less than about 75 kDa (e.g., less than about 70 kDa, about 65 kDa, about 60 kDa, about 55 kDa, or about 50 kDa). In certain embodiments, the weight average molecular weight of the polymer is between 3,000 and 1,000,000, between 3,000 and 100,000, between 3,000 and 10,000, between 10,000 and 1,000,000, between 10,000 and 100,000, or between 100,000 and 1,000,000, inclusive, g/mol. In certain embodiments, the weight average molecular weight of the polymer is between 10,000 and 30,000, between 30,000 and 100,000, between 100,000 and 300,000, or between 300,000 and 1,000,000, inclusive, g/mol. In certain embodiments, the weight average molecular weight of the polymer is between 30,000 and 300,000, inclusive, g/mol.

In some cases, the Brush prodrugs are of the form of particles (e.g., nanoparticles, i.e., the particle have a characteristic dimension of less than about 1 micrometer). In certain embodiments, the characteristic dimension of a particle is the diameter of a perfect sphere having the same volume as the particle. In certain embodiments, the Brush prodrug particle has a characteristic dimension of less than about 300 nm. In certain embodiments, the Brush prodrug particle has a characteristic dimension of less than about 200 nm. In certain embodiments, the Brush prodrug particle has a characteristic dimension of less than about 150 nm. In certain embodiments, the Brush prodrug particle has a characteristic dimension of less than about 100 nm. In certain embodiments, the Brush prodrug particle has a characteristic dimension of less than about 50 nm. In certain embodiments, the Brush prodrug particle has a characteristic dimension of less than about 30 nm. In certain embodiments, the Brush prodrug particle has a characteristic dimension of less than about 20 nm. In certain embodiments, the Brush prodrug particle has a characteristic dimension of less than about 10 nm. In certain embodiments, the Brush prodrug particle has a characteristic dimension between 6 and 250 nm, inclusive. In certain embodiments, the Brush prodrug particle has a characteristic dimension between 8 and 200 nm, inclusive. In certain embodiments, the Brush prodrug particle has a characteristic dimension between 12 and 200 nm, inclusive. In certain embodiments, the Brush prodrug particle has a characteristic dimension between 50 and 200 nm, inclusive. The term “average hydrodynamic diameter” as used herein refers to the average size of a Brush prodrug or particle. The average hydrodynamic diameter may or may not encompass the solvation layers of Brush prodrug or particle, and may be determined through a number of methods including dynamic light scattering, electron microscopy (e.g., scanning electron microscopy, transmission electron microscopy), atomic force microscopy, and X-ray diffraction. In some embodiments, the average hydrodynamic diameter of the Brush prodrug is less than 50 nm (e.g., less than about 45 nm, about 40 nm, about 35 nm, about 25 nm, about 20 nm, about 15 nm, about 10 nm, about 7.5 nm, or less), e.g., as determined by dynamic light scattering. In some embodiments, the average hydrodynamic diameter of the Brush prodrug is between about 1 nm and about 20 nm (e.g., between about 2.5 nm and about 17.5 nm, or about 5 nm and about 15 nm). In some embodiments, the average hydrodynamic diameter of the Brush prodrug is between about 5 nm and about 15 nm.

In some embodiments, the average hydrodynamic diameter of the particle is less than 100 nm (e.g., less than about 90 nm, about 80 nm, about 75 nm, about 70 nm, about 65 nm, about 60 nm, about 55 nm, about 50 nm, about 45 nm, about 40 nm, about 35 nm, about 25 nm, or less). e.g., as determined by dynamic light scattering. In some embodiments, the average hydrodynamic diameter of the particle is between about 5 nm and about 100 nm (e.g., between about 7.5 nm and about 75 nm, about 10 nm and about 50 nm, about 12.5 nm and about 40 nm, or about 15 nm and about 30 nm). In some embodiments, the average hydrodynamic diameter of the particle is between about 10 nm and about 50 nm. In some embodiments, the average hydrodynamic diameter of the particle is between about 15 nm and about 30 nm.

The term “average polydispersity” as used herein refers to a measure of the distribution of molecular size in a mixture, e.g., as determined by a chromatographic method, such as gel permeation chromatography or size exclusion chromatography, or through dynamic light scattering. In some embodiments, the average polydispersity of the Brush prodrug or particle is less than about 0.5 (e.g., less than about 0.4, about 0.35, about 0.3, about 0.25, about 0.2, about 0.15, or less). In some embodiments, the average polydispersity of the Brush prodrug or particle is less than about 0.3. In some embodiments, the average polydispersity of the Brush prodrug or particle is less than about 0.2. In some embodiments, the average polydispersity of the Brush prodrug or particle is less than about 0.15. In some embodiments, the Brush prodrug or particle is monodisperse. In some embodiments, the Brush prodrug or particle is about 50% monodisperse (e.g., about 55%, about 60%, about 65%, about 70%, about 75%, about 80%, about 85%, about 90%, about 95%, about 99%, or about 99.9% monodisperse).

In some embodiments, the Brush prodrug or particle is substantially soluble in water (e.g., hydrophilic). In some embodiments, the Brush prodrug or particle is substantially insoluble in water (e.g., hydrophobic). In some embodiments, the Brush prodrug or particle is substantially insoluble in water and greater than about 10,000 parts water are required to dissolve 1 part polymer. In one embodiment, the Brush prodrug or particle is amphiphilic. In one embodiment, the Brush prodrug or particle comprises a segment that is hydrophobic and a segment that is hydrophilic.

### Pharmaceutical Compositions and Kits

The present disclosure provides compositions (e.g., pharmaceutical compositions) comprising a polymer described herein, and optionally an excipient (e.g., pharmaceutically acceptable excipient). The present disclosure also provides compositions (e.g., pharmaceutical compositions) comprising a conjugate described herein, and optionally an excipient (e.g., pharmaceutically acceptable excipient). In certain embodiments, the pharmaceutical composition described herein comprises a polymer described herein and a pharmaceutically acceptable excipient. In certain embodiments, the pharmaceutical composition described herein comprises a conjugate described herein and a pharmaceutically acceptable excipient.

In certain embodiments, the pharmaceutical compositions are useful for delivering an agent (e.g., to a subject or cell). In certain embodiments, the pharmaceutical compositions are useful for treating a disease in a subject in need thereof. In certain embodiments, the pharmaceutical compositions are useful for preventing a disease in a subject.

In certain embodiments, the polymer or conjugate described herein is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a proliferative disease in a subject in need thereof. In certain embodiments, the effective amount is an amount effective for preventing a proliferative disease in a subject in need thereof. In certain embodiments, the effective amount is an amount effective for treating a hematological disease in a subject in need thereof. In certain embodiments, the effective amount is an amount effective for preventing a hematological disease in a subject in need thereof. In certain embodiments, the effective amount is an amount effective for treating a neurological disease in a subject in need thereof. In certain embodiments, the effective amount is an amount effective for preventing a neurological disease in a subject in need thereof. In certain embodiments, the effective amount is an amount effective for treating a in a painful condition subject in need thereof. In certain embodiments, the effective amount is an amount effective for preventing a painful condition in a subject in need thereof. In certain embodiments, the effective amount is an amount effective for treating a psychiatric disorder in a subject in need thereof. In certain embodiments, the effective amount is an amount effective for preventing a psychiatric disorder in a subject in need thereof. In certain embodiments, the effective amount is an amount effective for treating a metabolic disorder in a subject in need thereof. In certain embodiments, the effective amount is an amount effective for preventing a metabolic disorder in a subject in need thereof. In certain embodiments, the effective amount is an amount effective for reducing the risk of developing a disease (e.g., proliferative disease, hematological disease, neurological disease, painful condition, psychiatric disorder, or metabolic disorder) in a subject in need thereof. In certain embodiments, the effective amount is an amount effective for inhibiting the activity (e.g., aberrant activity, such as increased activity) of a protein kinase in a subject or cell.

In certain embodiments, the cell is in vitro. In certain embodiments, the cell is in vivo.

Pharmaceutical compositions described herein can be prepared by any method known in the art of pharmacology. In general, such preparatory methods include bringing the polymer or conjugate described herein (which may includes a therapeutic agent (the “active ingredient”)) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan monostearate (Tween® 60), polyoxyethylene sorbitan monooleate (Tween® 80), sorbitan monopalmitate (Span® 40), sorbitan monostearate (Spano 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brijo 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronice F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol. (d) disintegrating agents such as agar, calcium carbonate, potato or tapioca starch, alginic acid, certain silicates, and sodium carbonate. (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a polymer or conjugate described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the polymer or conjugate in powder form through the outer layers of the skin to the dermis are suitable.

Formulations suitable for topical administration include liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Polymers provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The polymers, conjugates, and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the polymer, conjugate, or pharmaceutical composition described herein is suitable for topical administration to the eye of a subject.

The exact amount of a polymer or conjugate required to achieve an effective amount will vary from subject to subject, depending, for example, on species, age, and general condition of a subject, severity of the side effects or disorder, identity of the particular polymer or conjugate, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a polymer or conjugate described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a polymer or conjugate described herein. In certain embodiments, a dose described herein includes independently between 1 mg and 3 mg, inclusive, of a polymer or conjugate described herein. In certain embodiments, a dose described herein includes independently between 3 mg and 10 mg, inclusive, of a polymer or conjugate described herein. In certain embodiments, a dose described herein includes independently between 10 mg and 30 mg, inclusive, of a polymer or conjugate described herein. In certain embodiments, a dose described herein includes independently between 30 mg and 100 mg, inclusive, of a polymer or conjugate described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

A polymer, conjugate, or composition, as described herein, can be administered in combination with one or more additional pharmaceutical agents (e.g., therapeutically and/or prophylactically active agents). The polymers, conjugates, or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of a protein kinase in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a polymer or conjugate described herein and an additional pharmaceutical agent shows a synergistic effect that is absent in a pharmaceutical composition including one of the polymer/conjugate and the additional pharmaceutical agent, but not both.

The polymer, conjugate, or composition can be administered concurrently with, prior to, or subsequent to one or more additional pharmaceutical agents, which are different from the polymer, conjugate, or composition and may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the polymer, conjugate, or composition described herein in a single dose or administered separately in different doses. The particular combination to employ in a regimen will take into account compatibility of the polymer or conjugate described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include anti-proliferative agents, anti-cancer agents, cytotoxic agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is a binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the polymers or conjugates described herein or pharmaceutical compositions can be administered in combination with an anti-cancer therapy including surgery, radiation therapy, transplantation (e.g., stem cell transplantation, bone marrow transplantation), immunotherapy, and chemotherapy.

Also encompassed by the disclosure are kits (e.g., pharmaceutical packs). In certain embodiments, the kits comprise: a macromonomer, or a salt thereof, a Brush prodrug, or a pharmaceutical composition; and instructions for using the macromonomer, or a salt thereof, the polymer, conjugate, or the pharmaceutical composition.

In certain embodiments, the kits comprise: a compound, or a salt thereof; and instructions for using the compound or the salt. In certain embodiments, the kits comprise: a compound; and instructions for using the compound.

In certain embodiments, the kits comprise: a conjugate, or a salt thereof, or a pharmaceutical composition; and instructions for using the conjugate, or a salt thereof, or the pharmaceutical composition.

The kits provided may comprise a pharmaceutical composition, conjugate, or polymer described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition, conjugate, or polymer described herein. In some embodiments, the pharmaceutical composition, conjugate, or polymer described herein provided in the first container and the second container are combined to form one unit dosage form.

In some embodiments, the percentage of the conjugates (e.g., in a particle) that comprise an agent is between about 1 and about 100% (e.g., about 1%, about 2%, about 3%, about 4%, about 5%, about 10%, about 15%, about 20%, about 30%, about 40%, about 50%, about 60%, about 70%, about 80%, about 90%, or about 100%). In some embodiments, the percentage of the conjugates that comprise an agent is less than about 50%, e.g., less than about 40%, less than about 35%, less than about 30%, less than about 25%, less than about 20%, less than about 15%, or less than about 10%. In some embodiments, the percentage of the conjugates (e.g., in a particle) that comprise an agent is between about 5% and about 50%, about 5% and about 40%, about 5% and about 30%, about 5% and about 25%, or about 5% and about 20%. In some embodiments, the percentage of the conjugates (e.g., in a particle) that comprise an agent is between about 5% and 90%. In some embodiments, the percentage of the conjugates (e.g., in a particle) that comprise an agent is between about 5% and about 75%. In the some embodiments, the conjugates (e.g., in a particle) that comprise an agent is between about 5% and about 50%. In the some embodiments, the percentage of the conjugates (e.g., in a particle) that comprise an agent is between about 10% and about 25%.

In some embodiments, the total amount of the agent present in the Brush prodrug or particle is greater than about 5% (e.g., about 6%, about 7%, about 8%, about 9%, about 10%, about 12%, about 15%, about 20%, about 25%, about 30%, or more) of the total size or weight of the Brush prodrug or particle. In some embodiments, the total amount of the agent present in the Brush prodrug or particle is greater than about 10% (e.g., about 12%, about 15%, about 20%, about 25%, about 30%, or more) of the total size or weight of the Brush prodrug or particle.

Without being bound by theory, the conjugates or particles disclosed herein may improve the efficiency of an agent by one or more of increasing the localization and/or release (e.g., preferential release) of the agent to a target cell (e.g., a cancer or a fibrotic cell; a cell associated with a hypoxic environment), or increasing the half life of the agent, thus resulting in a significantly higher amount of a released agent at a target site (e.g., a tumor or liver (e.g., cirrhotic cell). According, the conjugates and particles disclosed herein can be more effective therapeutically than the free agent (e.g., due to enhanced drug uptake in the target tissue) and/or allow for a lower therapeutic dose of the agent, e.g., without substantially compromising the resulting drug concentration at a target tissue. In some embodiments, the conjugates and particles disclosed herein can reduce the adverse effect associated with systemic administration of an agent in free form (e.g., not coupled to a polymer, conjugate, Brush prodrug or particle described herein).

Without being bound by theory, due to the localized delivery of the compositions described herein (e.g., the agent-containing particles), a lower dose or amount of the agent in the particles can be administered (e.g., through local sustained delivery) compared to the agent in free form. In other embodiments, the agent-containing particles are administered at a dose or amount of the agent that is less than the dose or amount of said agent in free form to have a desired effect (e.g., a desired therapeutic effect).

In some embodiments, the agent is incorporated into a particle at a dose that is less than the dose or amount of said agent in free form to have a desired effect (e.g., a desired therapeutic effect), e.g., the standard of care dose for the intended use of the free agent. In one embodiment, the agent are incorporated into the particles at a dose or amount of the agent that is less than the standard of care dose of the agent for a desired therapy (e.g., a dose that is less than about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, or about 0.95 that of the standard of care dose of the agent).

In some embodiments, the agent is incorporated into a particle at a dose equivalent to the dose or amount of said agent in free form to have a desired effect (e.g., a desired therapeutic effect), e.g., the standard of care dose for the intended use of the free agent. In these embodiments, the particle produces a greater therapeutic effect and/or a less adverse effect than the free agent. In certain embodiments, the particle increases the amount of the agent delivered to a tissue or cell in need thereof and reduces the amount of the agent exposed to a non-target tissue or cell, as compared to the free agent.

In some embodiments, the agent is incorporated into a particle at a dose higher than the dose or amount of said agent in free form to have a desired effect (e.g., a desired therapeutic effect), e.g., the standard of care dose for the intended use of the free agent. In some embodiments, the agent is incorporated into a particle at a dose higher than the dose or amount of said agent in free form that would produce an adverse effect by systemic administration (e.g., a reduction in blood pressure). In some embodiments, since the particle described herein releases the agent at a target site based on pH microenvironment, other non-target sites (e.g., blood vessels) with different pH would be less likely to be exposed to the agent.

In another aspect, provided are kits including a first container comprising a polymer or pharmaceutical composition described herein. In certain embodiments, the kit further comprises instructions for using the polymer or pharmaceutical composition.

In another aspect, provided are kits including a first container comprising a compound described herein. In certain embodiments, the kit further comprises instructions for using the compound.

In another aspect, provided are kits including a first container comprising a conjugate, or a salt thereof, or pharmaceutical composition described herein. In certain embodiments, the kit further comprises instructions for using the conjugate or pharmaceutical composition.

In certain embodiments, the kits are useful for delivering an agent (e.g., to a subject or cell). In certain embodiments, the kits are useful for treating a disease (e.g., proliferative disease, hematological disease, neurological disease, painful condition, psychiatric disorder, or metabolic disorder) in a subject in need thereof. In certain embodiments, the kits are useful for preventing a disease (e.g., proliferative disease, hematological disease, neurological disease, painful condition, psychiatric disorder, or metabolic disorder) in a subject in need thereof. In certain embodiments, the kits are useful for reducing the risk of developing a disease (e.g., proliferative disease, hematological disease, neurological disease, painful condition, psychiatric disorder, or metabolic disorder) in a subject in need thereof. In certain embodiments, the kits are useful for inhibiting the activity (e.g., aberrant activity, such as increased activity) of a protein kinase in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for delivering an agent. In certain embodiments, the kits and instructions provide for treating a disease (e.g., proliferative disease, hematological disease, neurological disease, painful condition, psychiatric disorder, or metabolic disorder) in a subject in need thereof. In certain embodiments, the kits and instructions provide for preventing a disease (e.g., proliferative disease, hematological disease, neurological disease, painful condition, psychiatric disorder, or metabolic disorder) in a subject in need thereof. In certain embodiments, the kits and instructions provide for reducing the risk of developing a disease (e.g., proliferative disease, hematological disease, neurological disease, painful condition, psychiatric disorder, or metabolic disorder) in a subject in need thereof. In certain embodiments, the kits and instructions provide for inhibiting the activity (e.g., aberrant activity, such as increased activity) of a protein kinase in a subject or cell. A kit described herein may include one or more additional pharmaceutical agents described herein as a separate composition.

### Methods of Use and Uses

The present disclosure also provides methods of using the polymers described herein, or a pharmaceutical composition thereof, for delivering an agent. The present disclosure also provides methods of using the polymers described herein, or a pharmaceutical composition thereof, for the treatment or prevention of a disease. In certain embodiments, the disease is a proliferative disease, hematological disease, neurological disease, painful condition, psychiatric disorder, or metabolic disorder. In certain embodiments, the disease is cancer (e.g., lung cancer, large bowel cancer, pancreas cancer, biliary tract cancer, or endometrial cancer), benign neoplasm, angiogenesis, inflammatory disease, autoinflammatory disease, or autoimmune disease. In certain embodiments, the polymers are useful for the treatment or prevention of the disease in part because at least one instance of the pharmaceutical agents included in the polymers are useful for the treatment or prevention of the disease. In certain embodiments, the polymers are advantageous over the at least one instance of the pharmaceutical agents for the treatment or prevention of the disease in part because the polymers improve (e.g., increase) the delivery of the at least one instance of the pharmaceutical agents to the subject (e.g., to the target organ, tissue, or cell of the subject).

In another aspect, the present disclosure provides methods of delivering a pharmaceutical agent to a subject in need thereof comprising administering to the subject in need thereof a polymer or a pharmaceutical composition.

In another aspect, the present disclosure provides methods of delivering a pharmaceutical agent to a cell comprising contacting the cell with a polymer or a pharmaceutical composition.

In another aspect, the present disclosure provides methods of treating a disease in a subject in need thereof comprising administering to or implanting in the subject in need thereof a therapeutically effective amount of: a polymer or a pharmaceutical composition; wherein at least one instance of the pharmaceutical agent is a therapeutic agent.

In another aspect, the present disclosure provides methods of preventing a disease in a subject in need thereof comprising administering to or implanting in the subject in need thereof a prophylactically effective amount of: a polymer or a pharmaceutical composition; wherein at least one instance of the pharmaceutical agent is a prophylactic agent.

In another aspect, the present disclosure provides methods of diagnosing a disease in a subject comprising administering to or implanting in the subject a diagnostically effective amount of: a polymer or a pharmaceutical composition; wherein at least one instance of the pharmaceutical agent is a diagnostic agent. In certain embodiments, the polymers are useful for diagnosing the disease in part because at least one instance of the pharmaceutical agents included in the polymers are useful for diagnosing the disease. In certain embodiments, the polymers are advantageous over the at least one instance of the pharmaceutical agents for diagnosing the disease in part because the polymers improve (e.g., increase) the delivery of the at least one instance of the pharmaceutical agents to the subject (e.g., to the target organ, tissue, or cell of the subject).

The present disclosure also provides methods of using the conjugates described herein, or a pharmaceutical composition thereof, for delivering an agent. The present disclosure also provides methods of using the conjugates described herein, or a pharmaceutical composition thereof, for the treatment or prevention of a disease.

In certain embodiments, the disease is a proliferative disease, hematological disease, neurological disease, painful condition, psychiatric disorder, or metabolic disorder. In certain embodiments, the disease is a proliferative disease. In certain embodiments, the disease is a proliferative disease, and at least one instance of the pharmaceutical agent is an anti-proliferative agent. In certain embodiments, the disease is cancer (e.g., lung cancer, large bowel cancer, pancreas cancer, biliary tract cancer, or endometrial cancer), benign neoplasm, angiogenesis, inflammatory disease, autoinflammatory disease, or autoimmune disease. In certain embodiments, the disease is cancer. In certain embodiments, the disease is cancer, and at least one instance of the pharmaceutical agent is an anti-cancer agent.

In another aspect, the present disclosure provides methods of delivering a pharmaceutical agent to a subject in need thereof comprising administering to the subject in need thereof a conjugate or a pharmaceutical composition.

In another aspect, the present disclosure provides methods of delivering a pharmaceutical agent to a cell comprising contacting the cell with a conjugate or a pharmaceutical composition.

In another aspect, the present disclosure provides methods of treating a disease in a subject in need thereof comprising administering to or implanting in the subject in need thereof a therapeutically effective amount of: a conjugate or a pharmaceutical composition; wherein at least one instance of the pharmaceutical agent is a therapeutic agent.

In another aspect, the present disclosure provides methods of preventing a disease in a subject in need thereof comprising administering to or implanting in the subject in need thereof a prophylactically effective amount of: a conjugate or a pharmaceutical composition; wherein at least one instance of the pharmaceutical agent is a prophylactic agent.

In another aspect, the present disclosure provides methods of diagnosing a disease in a subject comprising administering to or implanting in the subject a diagnostically effective amount of: a conjugate or a pharmaceutical composition; wherein at least one instance of the pharmaceutical agent is a diagnostic agent.

In certain embodiments, the conjugates are useful for treating, preventing, or diagnosing the disease in part because at least one instance of the pharmaceutical agents included in the conjugates are useful for treating, preventing, or diagnosing the disease. In certain embodiments, the conjugates are advantageous over the at least one instance of the pharmaceutical agents for treating, preventing, or diagnosing the disease in part because the conjugates improve (e.g., increase) the delivery of the at least one instance of the pharmaceutical agents to the subject (e.g., to the target organ, tissue, or cell of the subject).

In some embodiments, the polymers or conjugates described herein, or a pharmaceutical composition thereof are useful in treating a cancer. In some embodiments, the polymers or conjugates described herein, or a pharmaceutical composition thereof, are useful to delay the onset of, slow the progression of, or ameliorate the symptoms of cancer. In some embodiments, the polymers or conjugates described herein, or a pharmaceutical composition thereof, are administered in combination with other compounds, drugs, or therapeutics to treat cancer.

In some embodiments, the polymers or conjugates described herein, or a pharmaceutical composition thereof are useful for treating a cancer including, but not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenström's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM), a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendocrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In some embodiments, the polymers or conjugates described herein, or a pharmaceutical composition thereof, are useful in treating lung cancer, head-and-neck cancer, esophagus cancer, stomach cancer, breast cancer, pancreas cancer, liver cancer, kidney cancer, prostate caner, glioblastomas, metastatic melanomas, peritoneal or pleural mesotheliomas.

In some embodiments, the proliferative disease is a benign neoplasm. All types of benign neoplasms disclosed herein or known in the art are contemplated as being within the scope of the invention. In some embodiments, the proliferative disease is associated with angiogenesis. All types of angiogenesis disclosed herein or known in the art are contemplated as being within the scope of the invention. In certain embodiments, the proliferative disease is an inflammatory disease. All types of inflammatory diseases disclosed herein or known in the art are contemplated as being within the scope of the invention. In certain embodiments, the inflammatory disease is rheumatoid arthritis. In some embodiments, the proliferative disease is an autoinflammatory disease. All types of autoinflammatory diseases disclosed herein or known in the art are contemplated as being within the scope of the invention. In some embodiments, the proliferative disease is an autoimmune disease. All types of autoimmune diseases disclosed herein or known in the art are contemplated as being within the scope of the invention.

In certain embodiments, the methods described herein include administering to a subject with an effective amount of the polymers or conjugates described herein, or a pharmaceutical composition thereof. In certain embodiments, the methods described herein include implanting to a subject with an effective amount of the polymers or conjugates described herein, or a pharmaceutical composition thereof.

In certain embodiments, the polymers or conjugates described herein, or a pharmaceutical composition thereof, are administered in combination with one or more additional pharmaceutical agents described herein. In certain embodiments, the additional pharmaceutical agent is an anti-cancer agent. Anti-cancer agents encompass biotherapeutic anti-cancer agents as well as chemotherapeutic agents. Exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g., HERCEPTIN (trastuzumab), T-DM1, AVASTIN (bevacizumab), ERBITUX (cetuximab), VECTIBIX (panitumumab), RITUXAN (rituximab), BEXXAR (tositumomab)). Exemplary chemotherapeutic agents include, but are not limited to, anti-estrogens (e.g., tamoxifen, raloxifene, and megestrol), LHRH agonists (e.g., goscrclin and leuprolide), anti-androgens (e.g., flutamide and bicalutamide), photodynamic therapies (e.g., vertoporfin (BPD-MA), phthalocyanine, photosensitizer Pc4, and demethoxy-hypocrellin A (2BA-2-DMHA)), nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g., dacarbazine, temozolomide), platinum containing compounds (e.g., cisplatin, carboplatin, oxaliplatin), vinca alkaloids (e.g., vincristine, vinblastine, vindesine, and vinorelbine), taxoids (e.g., paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g., etoposide, etoposide phosphate, teniposide, topotecan, 9-aminocamptothecin, camptoirinotecan, irinotecan, crisnatol, mytomycin C), anti-metabolites, DHFR inhibitors (e.g., methotrexate, dichloromethotrexate, trimetrexate, edatrexate), IMP dehydrogenase inhibitors (e.g., mycophenolic acid, tiazofurin, ribavirin, and EICAR), ribonuclotide reductase inhibitors (e.g., hydroxyurea and deferoxamine), uracil analogs (e.g., 5-fluorouracil (5-FU), floxuridine, doxifluridine, ratitrexed, tegafur-uracil, capecitabine), cytosine analogs (e.g., cytarabine (ara C), cytosine arabinoside, and fludarabine), purine analogs (e.g., mercaptopurine and Thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone), MDR inhibitors (e.g., verapamil), Ca²⁺ ATPase inhibitors (e.g., thapsigargin), imatinib, thalidomide, lenalidomide, tyrosine kinase inhibitors (e.g., axitinib (AGO13736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT, SUI1248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

### EXAMPLES

In order that the invention described herein may be more fully understood, the following examples are set forth. The synthetic and biological examples described herein are offered to illustrate the present disclosure and are not to be construed in any way as limiting their scope.

### Preparation and Characterization of Exemplary Macromonomers, Compounds, and Polymers Described Herein

Certain synthetic intermediates were or can be prepared according to reported methods, such as methods described in U.S. Pat. Nos. 6,812,238 and 10,716,85; U.S. patent application publications, 2014/0308234 and 2017/0348431; international PCT applications, PCT/US2017/064784, filed Dec. 5, 2017 and published as WO 2018/106738, and PCT/US2019/027414, filed Apr. 13, 2019 and published as WO 2019/200367; U.S. provisional patent applications, 62/528,010, filed Jun. 30, 2017, and 62/520,473, filed Jun. 15, 2017; Ohwada et al., Bioorganic & Medicinal Chemistry Letters, Volume 13, Issue 2, 2003, pages 191-196; the entire contents of each of which are incorporated herein by reference.

### Example 1.1. Synthesis of JQ-N₃ (JQ1-Loaded)

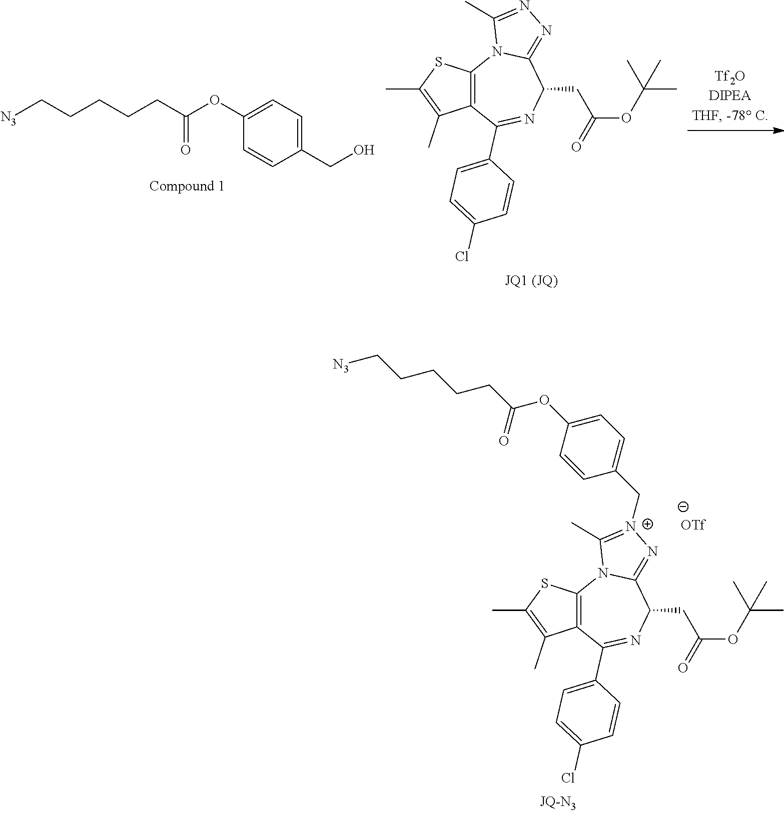 [SMILES: [N-]=[N+]=NCCCCCC(=O)Oc1ccc(CO)cc1 ; Cc1sc2c(c1C)C(c1ccc(Cl)cc1)=N[C@@H](CC(=O)OC(C)(C)C)c1nnc(C)n1-2 ; Cc1sc2c(c1C)C(c1ccc(Cl)cc1)=N[C@@H](CC(=O)OC(C)(C)C)c1n[n+](Cc3ccc(OC(=O)CCCCCN=[N+]=[N-])cc3)c(C)n1-2 ; "OTf"]

To an oven dried N₂ filled vial, trifluoromethane sulfonic anhydride (108 mg, 0.380 mmol) was added. Anhydrous THF (1.20 mL) was then added to the vial. The solution was kept under N₂ and cooled to −78° C. A solution of Compound 1 (100 mg, 0.38 mmol) and DIPEA (52 mg, 0.38 mmol) in anhydrous THF (0.40 mL) was then added dropwise into the reaction mixture over 10 minutes. After complete addition, the reaction was left to react for 30 minutes at −78° C. At this point, a solution of JQ1 (176 mg, 0.38 mmol) in anhydrous THF (0.40 mL) was added dropwise to the reaction mixture over 10 minutes. After complete addition, the reaction was left at −78° C. for another hour, at which point MeOH (80 μL) was added to quench the reaction. The reaction mixture was concentrated and purified using a neutral alumina column (MeOH in DCM) to yield the product product as a solid (130 mg, 0.16 mmol, 43% yield). ¹H NMR (600 MHz, CDCl₃, ppm) δH 7.44 (d, 2H), 7.42 (d, 2H), 7.35 (d, 2H), 7.09 (d, 2H), 5.63 (dd, 2H), 4.71 (dd, 1H), 3.49 (dd, 1H), 3.40 (dd, 1H), 3.29 (t, 2H), 3.06 (s, 3H), 2.56 (t, 2H), 2.46 (s, 3H), 1.75 (p, 2H), 1.72 (s, 3H), 1.66 (m, 2H), 1.50-1.47 (overlap, 11H). ¹³C NMR (150 MHz, CDCl₃, ppm): δC 171.9, 169.2, 164.9, 154.6, 151.6, 150.8, 137.7, 136.0, 135.6, 133.7, 131.5, 130.4, 130.4, 130.1, 129.9, 129.1, 129.0, 122.8, 122.6, 81.8, 55.4, 53.0, 51.3, 37.1, 34.2, 28.6, 28.2, 28.1, 26.3, 24.4, 14.4, 13.5, 11.9.

### Example 1.2. Synthesis of JQ-N₃-2 (JQ1-Loaded)

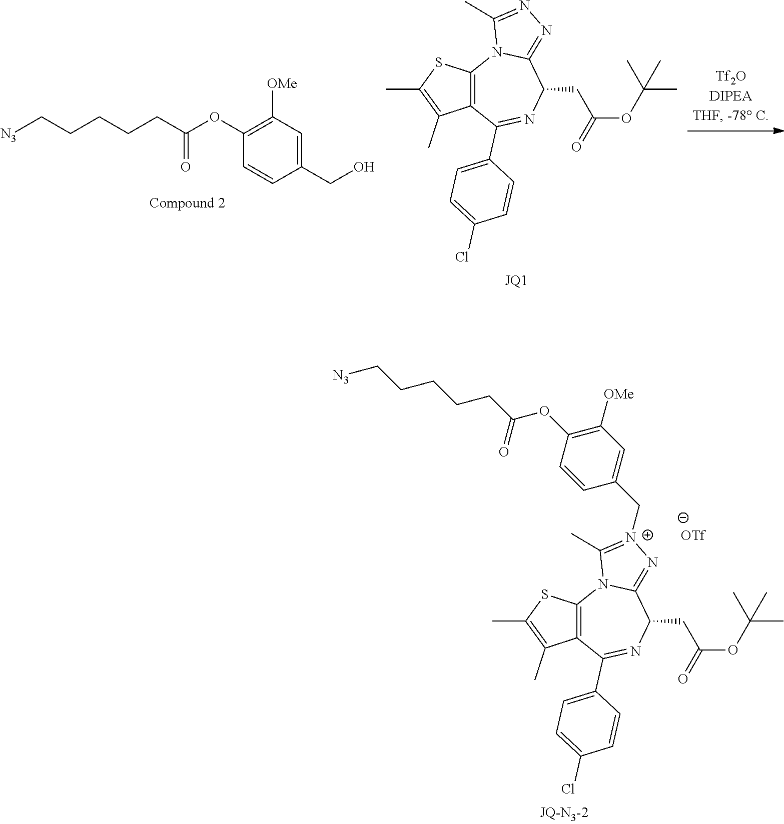 [SMILES: COc1cc(CO)ccc1OC(=O)CCCCCN=[N+]=[N-] ; Cc1sc2c(c1C)C(c1ccc(Cl)cc1)=N[C@@H](CC(=O)OC(C)(C)C)c1nnc(C)n1-2 ; COc1cc(C[n+]2nc3n(c2C)-c2sc(C)c(C)c2C(c2ccc(Cl)cc2)=N[C@H]3CC(=O)OC(C)(C)C)ccc1OC(=O)CCCCCN=[N+]=[N-] ; "OTf"]

To an oven dried N₂ filled vial, trifluoromethane sulfonic anhydride (54 mg, 0.19 mmol) was added. Anhydrous THF (0.60 mL) was then added to the vial. The solution was kept under N₂ and cooled to −78° C. A solution of Compound 2 (55 mg, 0.19 mmol) and DIPEA (26 mg, 0.19 mmol) in anhydrous THF (0.20 mL) was then added dropwise into the reaction mixture over 10 minutes. After complete addition, the reaction was left to react for 30 minutes at −78° C. At this point, a solution of JQ1 (88 mg, 0.19 mmol) in anhydrous THF (0.20 mL) was added dropwise to the reaction mixture over 10 minutes. After complete addition, the reaction was left at −78° C. for another hour, at which point MeOH (40 μL) was added to quench the reaction. The reaction mixture was concentrated and purified using a neutral alumina column (MeOH in DCM) to yield the product product as a solid (65 mg, 0.077 mmol, 40% yield). ¹H NMR (600 MHz, CDCl₃, ppm) δH 7.43 (d, 2H), 7.34 (d, 2H), 7.13 (s, 1H), 6.98 (d, 1H), 6.95 (d, 1H), 5.56 (s, 2H), 4.70 (dd, 1H), 3.82 (s, 3H), 3.47 (dd, 1H), 3.38 (dd, 1H), 3.28 (t, 2H), 3.06 (s, 3H), 2.56 (t, 2H), 2.45 (s, 3H), 1.74 (m, 2H), 1.70 (s, 3H), 1.64 (m, 2H), 1.50-1.46 (overlap, 11H). ¹³C NMR (150 MHz, CDCl₃, ppm): δC 171.5, 169.2, 164.9, 154.5, 151.8, 150.9, 140.7, 137.6, 135.9, 135.6, 133.7, 131.5, 130.2, 130.1, 129.0, 127.9, 123.6, 121.4, 113.6, 81.7, 56.3, 55.7, 53.0, 51.3, 37.1, 33.8, 28.6, 28.2, 28.2, 28.2, 26.2, 24.5, 14.4, 13.5, 11.8.

### Example 1.3. Synthesis of JQ-N₃-3 (JQ1-Loaded)

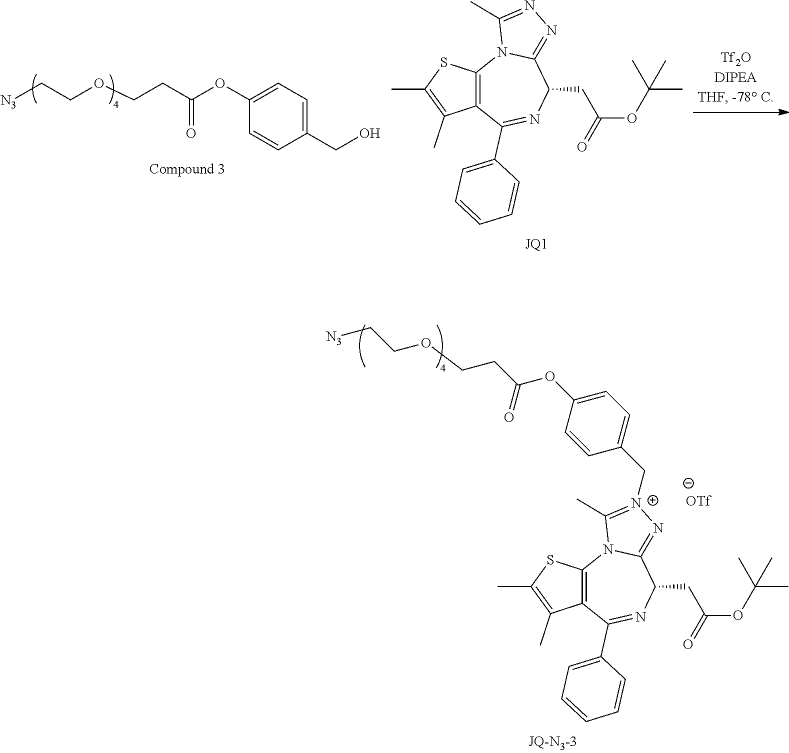

To an oven dried N₂ filled vial, trifluoromethane sulfonic anhydride (118 mg, 0.42 mmol) was added. Anhydrous THF (1.50 mL) was then added to the vial. The solution was kept under N₂ and cooled to −78° C. A solution of Compound 3 (150 mg, 0.42 mmol) and DIPEA (55 mg, 0.42 mmol) in anhydrous THF (0.60 mL) was then added dropwise into the reaction mixture over 10 minutes. After complete addition, the reaction was left to react for 30 minutes at −78° C. At this point, a solution of JQ1 (192 mg, 0.42 mmol) in anhydrous THF (0.60 mL) was added dropwise to the reaction mixture over 10 minutes. After complete addition, the reaction was left at −78° C. for another hour, at which point MeOH (150 μL) was added to quench the reaction. The reaction mixture was concentrated and purified using a neutral alumina column (MeOH in DCM) to yield the product as a solid (206 mg, 0.22 mmol, 52% yield). ¹H NMR (600 MHz, CDCl₃, ppm) δH 7.44-7.43 (overlap, 4H), 7.35 (d, 2H), 7.11 (d, 2H), 5.63 (q, 2H), 4.72 (dd, 1H), 3.84 (t, 2H), 3.67-3.62 (overlap, 14H), 3.50 (dd, 1H), 3.40 (dd, 1H), 3.37 (t, 2H), 3.10 (s, 3H), 2.82 (t, 2H), 2.46 (s, 3H), 1.72 (s, 1H), 1.50 (s, 9H).

### Example 1.4. Synthesis of AZ-N₃ (AZD5153-Loaded)

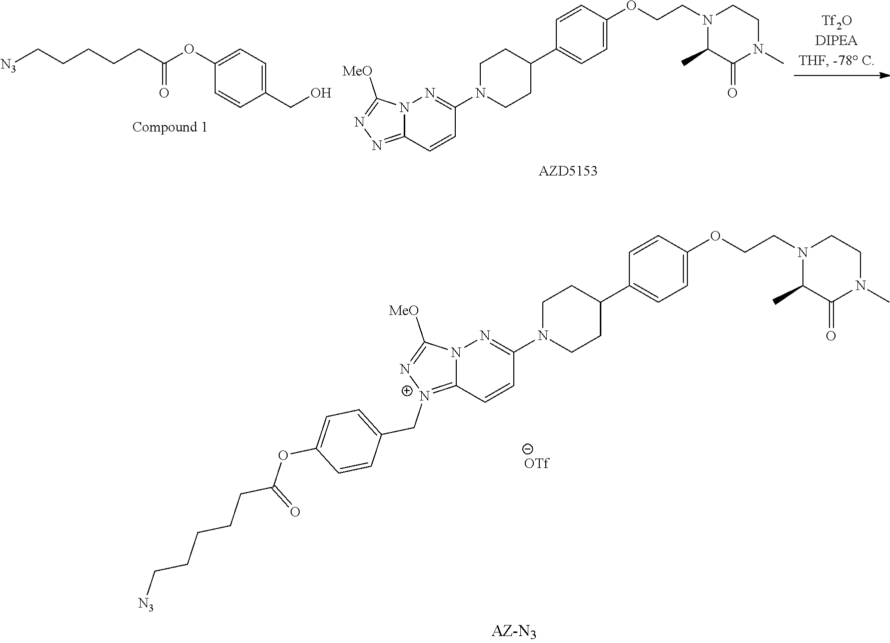 [SMILES: [N-]=[N+]=NCCCCCC(=O)Oc1ccc(CO)cc1 ; COc1nnc2ccc(N3CCC(c4ccc(OCCN5CCN(C)C(=O)[C@H]5C)cc4)CC3)nn12 ; COc1n[n+](Cc2ccc(OC(=O)CCCCCN=[N+]=[N-])cc2)c2ccc(N3CCC(c4ccc(OCCN5CCN(C)C(=O)[C@H]5C)cc4)CC3)nn12]

To an oven dried N₂ filled vial, trifluoromethane sulfonic anhydride (540. mg, 1.90 mmol) was added. Anhydrous THF (3.0 mL) was then added to the vial. The solution was kept under N₂ and cooled to −78° C. A solution of Compound 1 (500. mg, 1.90 mmol) and DIPEA (250 mg, 1.90 mmol) in anhydrous THF (0.50 mL) was then added dropwise into the reaction mixture over 10 minutes. After complete addition, the reaction was left to react for 30 minutes at −78° C. At this point, a solution of AZD5153 (830. mg, 1.70 mmol) in anhydrous THF (0.10 mL) was added dropwise to the reaction mixture over 10 minutes. After complete addition, the reaction was left at −78° C. for another hour, at which point MeOH (0.40 mL) was added to quench the reaction. The reaction mixture was concentrated and purified using a neutral alumina column (MeOH in DCM) to yield the product as a solid (530 mg, 0.61 mmol, 36% yield). LRMS: Calcd for C₃₉H₄₉N₁₀O₅: m/z=725.4; Found: 725.3 [M−OTf]⁺. ¹H NMR (600 MHz, CDCl₃, ppm) δH 9.15 (d, 1H), 7.77 (d, 1H), 7.65 (d, 2H), 7.09 (dd, 4H), 6.83 (d, 2H), 5.84 (s, 2H), 4.43 (br, 1H), 4.41 (br, 1H), 4.35 (s, 3H), 4.06 (br, 2H), 3.34 (br, 2H), 3.32-3.25 (overlap, 4H), 3.14 (overlap, 3H), 3.03 (overlap, 2H), 2.94 (s, 3H), 2.87 (br, 1H), 2.81-2.75 (overlap, 2H), 2.58 (t, 2H), 2.17 (s, 2H), 1.98 (br, 2H), 1.80-1.63 (overlap, 6H), 1.50 (m, 2H), 1.40 (d, 3H). ¹³C NMR (150 MHz, CDCl3, ppm): δC 171.9, 170.59, 157.4, 155.8, 151.9, 151.5, 137.1, 136.8, 130.5, 130.3, 127.8, 122.8, 122.6, 119.9, 114.8, 66.6, 60.6, 60.08, 54.2, 52.9, 51.3, 48.3, 46.8, 46.8, 41.5, 34.6, 34.2, 32.9, 28.6, 26.3, 24.4, 15.5.

### Example 1.5. Synthesis of Vin-N₃ (Vincristine-Loaded)

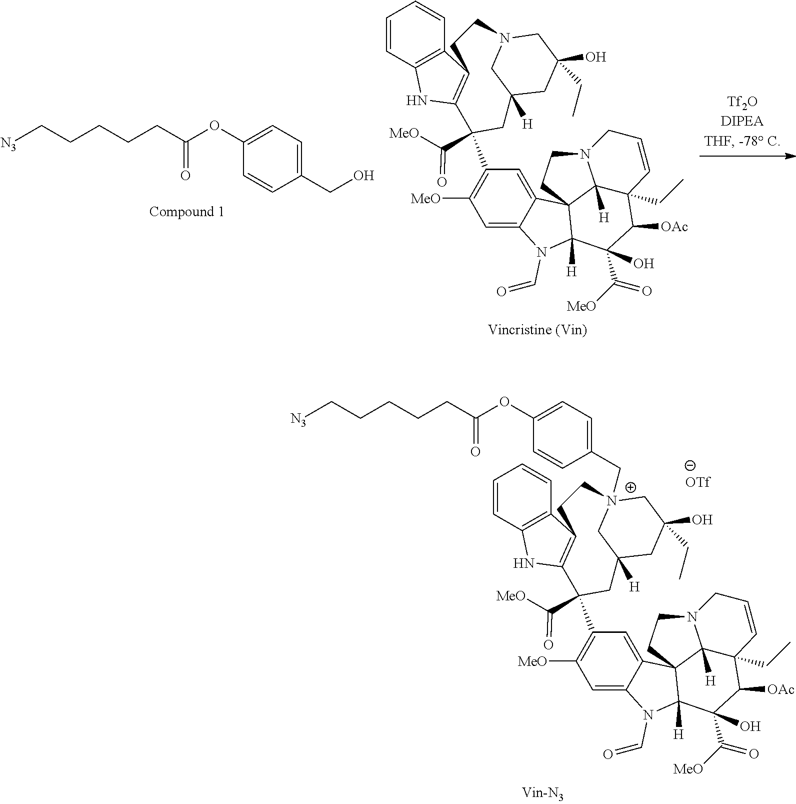

To an oven dried N₂ filled vial, trifluoromethane sulfonic anhydride (6.0 mg, 0.021 mmol) was added. Anhydrous THE (0.15 mL) was then added to the vial. The solution was kept under N₂ and cooled to −78° C. A solution of Compound 1 (5.2 mg, 0.021 mmol) and DIPEA (5.5 mg, 0.043 mmol) in anhydrous THF (0.05 mL) was then added dropwise into the reaction mixture over 10 minutes. After complete addition, the reaction was left to react for 30 minutes at −78° C. At this point, a solution of vincristine (20 mg, 0.021 mmol) in anhydrous THF (0.05 mL) was added dropwise to the reaction mixture over 10 minutes. After complete addition, the reaction was left at −78° C. for another hour, at which point MeOH (10 μL) was added to quench the reaction. The reaction mixture was concentrated and purified using a neutral alumina column (MeOH in DCM) to yield the product as a solid (13 mg, 0.012 mmol, 57% yield). LRMS: Calcd for C₅₉H₇₂N₇O₁₂: m/z=1071.3; Found: 1071.4 [M−OTf]⁺. ¹H NMR (600 MHz, CDCl₃, ppm) δH 9.42 (br, 1H), 8.76 (s, 1H), 8.16 (s, 1H), 8.04 (br, 2H), 7.74 (s, 1H), 7.55 (s, 1H), 7.54 (s, 1H), 7.21-7.12 (overlap, 6H), 6.91 (s, 1H), 6.79 (s, 1H), 5.91 (m, 2H), 5.41 (s, 1H), 5.39 (s, 1H), 5.25 (s, 1H), 5.21 (s, 1H), 4.74 (s, 1H), 4.51 (s, 1H), 4.00 (overlap, 2H), 3.90 (s, 3H), 3.88 (s, 3H), 3.78 (s, 3H), 3.72 (s, 3H), 3.67 (s, 6H), 3.40-3.25 (overlap, 8H), 3.19 (s, 3H), 3.16 (br, 2H), 2.93-2.88 (overlap, 4H), 2.81 (br, 3H), 2.62 (overlap, 2H), 2.36 (overlap, 4H), 2.15 (m, 1H), 2.10 (s, 2H), 2.06 (s, 3H), 1.75-1.60 (overlap), 1.50-1.28 (overlap), 0.91-0.84 (overlap, 12H).

### Example 1.6. Synthesis of JQ-MM (JQ1-Loaded)

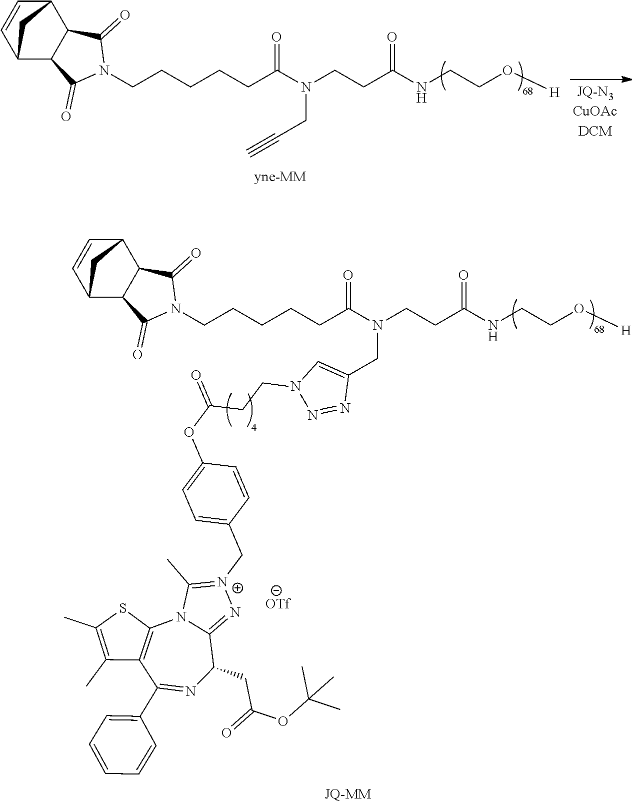

To a vial, yne-MM (67.7 mg, 0.020 mmol, 1.0 eq), JQ-N₃ (20.5 mg, 0.023 mmol, 1.15 eq), and DCM (4.0 mL) were added. CuOAc (a pinch) was then added and the reaction mixture was stirred under N₂ atmosphere. The reaction was complete in ˜1 h as determined by LC-MS. The crude mixture was ran through an aluminum oxide plug. The collected solution was concentrated under vacuum, redissolved in CHCl₃, filtered through a 0.45 μm filter (Nalgene), and subjected to recycling preparative HPLC. The fractions containing the product were concentrated under vacuum and dried overnight, affording the product as a solid (69.5 mg, 81% yield).

### Example 1.7. Synthesis of JQ-MM-2 (JQ1-Loaded)

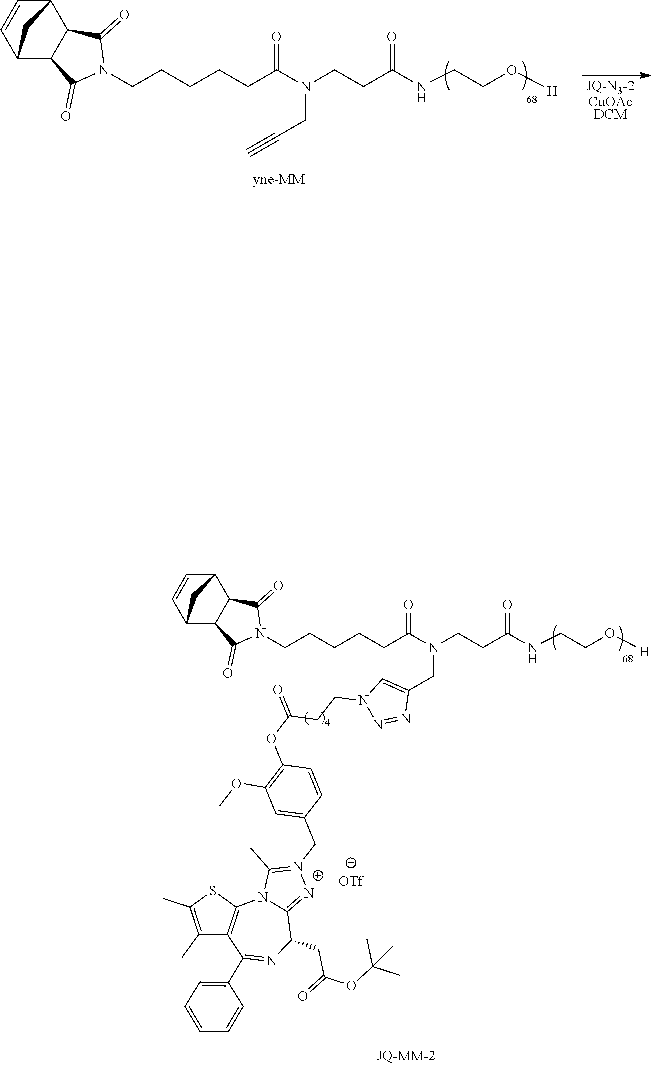

To a vial, yne-MM (175.3 mg, 0.052 mmol, 1.0 eq), JQ-N₃-2 (52.8 mg, 0.023 mmol, 1.15 eq), and DCM (5.0 mL) were added. CuOAc (a pinch) was then added and the reaction mixture was stirred under N₂ atmosphere. The reaction was complete in ˜1 h as determined by LC-MS. The crude mixture was ran through an aluminum oxide plug. The collected solution was concentrated under vacuum, redissolved in CHCl₃, filtered through a 0.45 μm filter (Nalgene), and subjected to recycling preparative HPLC. The fractions containing the product were concentrated under vacuum and dried overnight, affording the product as a solid (194.8 mg, 85.4% yield).

### Example 1.8. Synthesis of JQ-MM-3 (JQ1-Loaded)

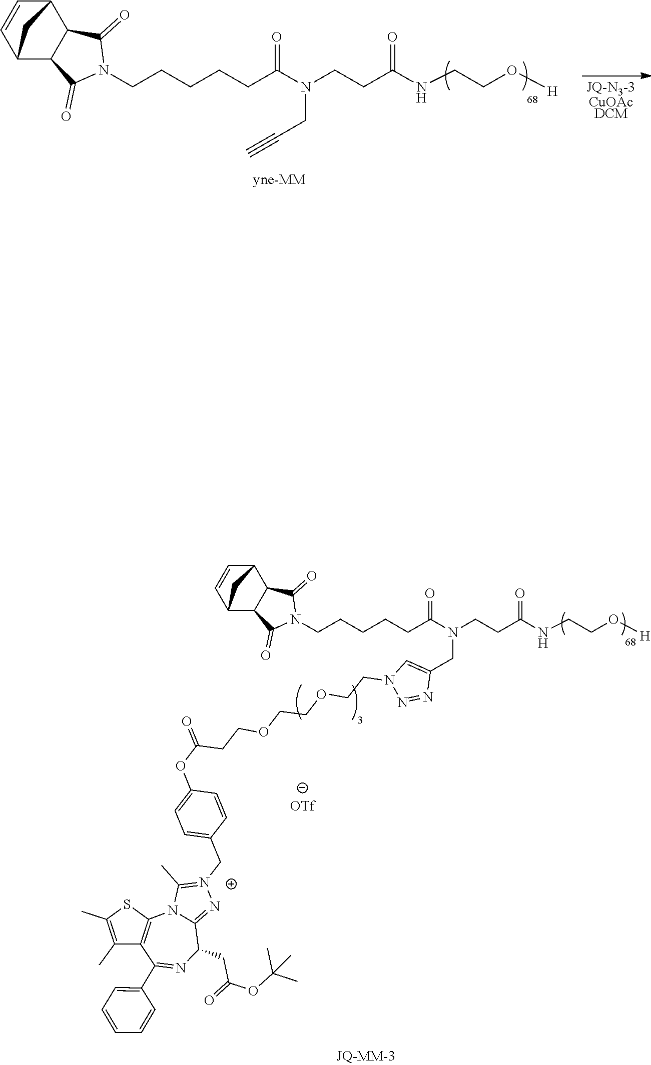

To a vial, yne-MM (251.2 mg, 0.075 mmol, 1.0 eq), JQ-N₄-3 (84.6 mg, 0.086 mmol, 1.15 eq), and DCM (12.0 mL) were added. CuOAc (a pinch) was then added and the reaction mixture was stirred under N₂ atmosphere. The reaction was complete in ˜1 h as determined by LC-MS. The crude mixture was ran through an aluminum oxide plug. The collected solution was concentrated under vacuum, redissolved in CHCl₃, filtered through a 0.45 μm filter (Nalgene), and subjected to recycling preparative HPLC. The fractions containing the product were concentrated under vacuum and dried overnight, affording the product as a solid (249.7 mg, 76.9% yield).

### Example 1.9. Synthesis of AZ-MM (AZ5153-Loaded)

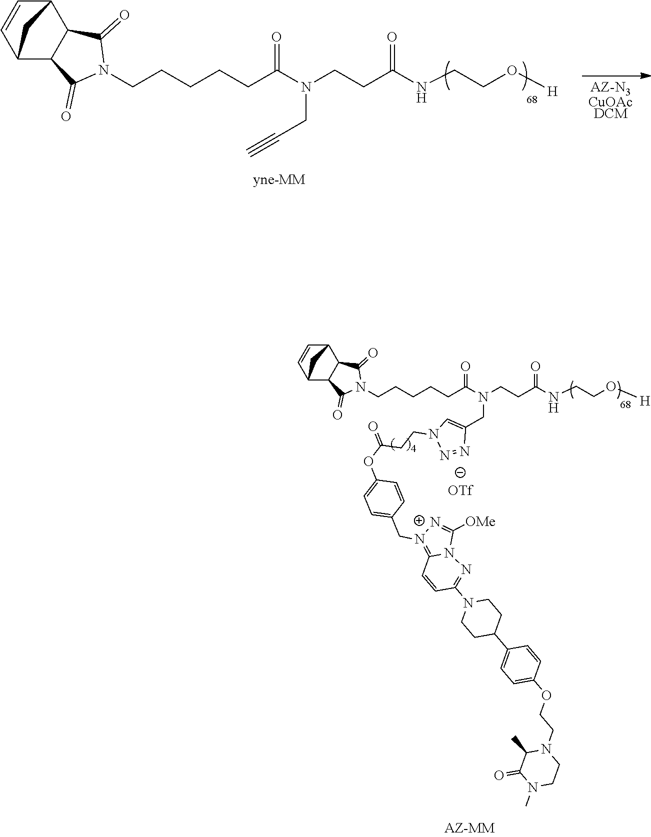

To a vial, yne-MM (93.7 mg, 0.028 mmol, 1.0 eq), AZ-N₃ (28.0 mg, 0.032 mmol, 1.15 eq), and DCM (4.0 mL) were added. CuOAc (a pinch) was then added and the reaction mixture was stirred under N₂ atmosphere. The reaction was complete in ˜1 h as determined by LC-MS. The crude mixture was ran through an aluminum oxide plug. The collected solution was concentrated under vacuum, redissolved in CHCl₃, filtered through a 0.45 μm filter (Nalgene), and subjected to recycling preparative HPLC. The fractions containing the product were concentrated under vacuum and dried overnight, affording the product as a solid (70.5 mg, 60% yield).

### Example 1.10. Synthesis of Vin-MM (Vincristine-Loaded)

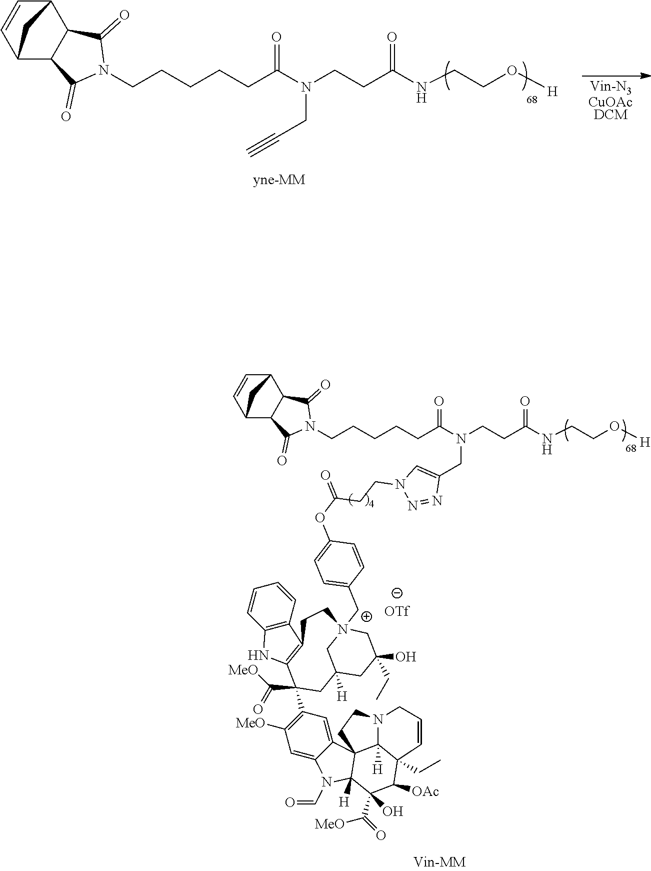

To a vial, yne-MM (32.2 mg, 9.57 μmol, 1.0 eq). Vin-N₃ (12.2 mg, 11.0 μmol, 1.15 eq), and DCM (2.0 mL) were added. CuOAc (a pinch) was then added and the reaction mixture was stirred under N₂ atmosphere. The reaction was complete in ˜1 h as determined by LC-MS. The crude mixture was ran through an aluminum oxide plug. The collected solution was concentrated under vacuum, redissolved in CHCl₃, filtered through a 0.45 μm filter (Nalgene), and subjected to recycling preparative HPLC. The fractions containing the product were concentrated under vacuum and dried overnight, affording the product as a solid (31.4 mg, 73% yield).

### Example 1.11. Synthesis of JQ-BBP

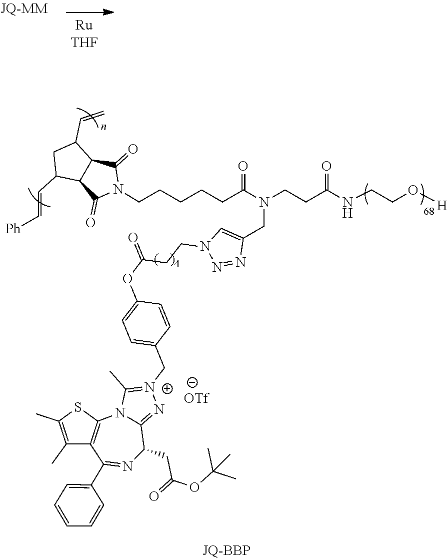

To a vial containing a stir bar, JQ-MM (21.3 mg, 5.00 μmol, 10.0 eq) was added. To another vial, a solution of 3^{rd} generation Grubbs catalyst (Ru, 0.02 M in THF) was freshly prepared. THF (75.0 μL) was then added to the vial containing JQ-MM, followed by the addition of Ru solution (25.0 μL, 0.50 μmol, 1.0 eq) to give the desired DP of 10, while achieving a total JQ-MM concentration of 0.05 M. The yellow reaction mixture was allowed to stir for 3 hours at room temperature. To quench the polymerization, a drop of ethyl vinyl ether was then added. The reaction mixture was transferred to 8 kDa molecular weight cutoff dialysis tubing in 3 mL nanopure water, and the solution was dialyzed against H₂O (500 mL×3, solvent exchange every 6 h). The dialyzed solution of JQ-BBP was then concentrated to the desired concentration via centrifugation with a filter tube. Alternatively, JQ-BBP can also be acquired by lyophilization.

### Example 1.12. Synthesis of JQ-BBP-2 (JQ1-Loaded)

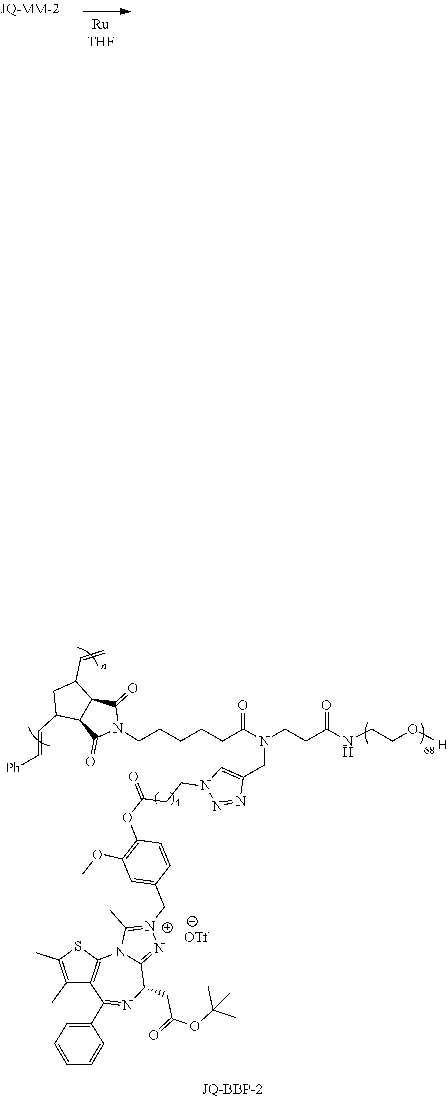

To a vial containing a stir bar, JQ-MM-2 (46.5 mg, 10.9 μmol, 10.0 eq) was added. To another vial, a solution of 3^{rd} generation Grubbs catalyst (Ru, 0.02 M in THF) was freshly prepared. THF (164.1 μL) was then added to the vial containing JQ-MM-2, followed by the addition of Ru solution (54.7 μL, 1.09 μmol, 1.0 eq) to give the desired DP of 10, while achieving a total JQ-MM-2 concentration of 0.05 M. The yellow reaction mixture was allowed to stir for 3 hours at room temperature. To quench the polymerization, a drop of ethyl vinyl ether was then added. The reaction mixture was transferred to 8 kDa molecular weight cutoff dialysis tubing in 3 mL nanopure water, and the solution was dialyzed against H₂O (500 mL×3, solvent exchange every 6 h). The dialyzed solution of JQ-BBP-2 was then concentrated to the desired concentration via centrifugation with a filter tube. Alternatively, JQ-BBP-2 can also be acquired by lyophilization.

### Example 1.13. Synthesis of JQ-BBP-3 (JQ1-Loaded)

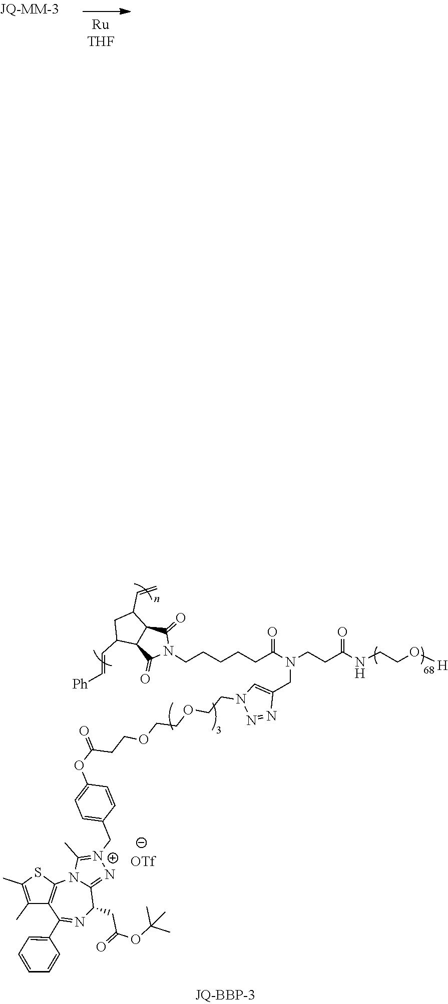

To a vial containing a stir bar, JQ-MM-3 (54.8 mg, 12.6 μmol, 10.0 eq) was added. To another vial, a solution of 3^{rd} generation Grubbs catalyst (Ru, 0.02 M in THF) was freshly prepared. THF (188.8 μL) was then added to the vial containing JQ-MM-3, followed by the addition of Ru solution (62.9 μL, 1.26 μmol, 1.0 eq) to give the desired DP of 10, while achieving a total JQ-MM-3 concentration of 0.05 M. The yellow reaction mixture was allowed to stir for 3 hours at room temperature. To quench the polymerization, a drop of ethyl vinyl ether was then added. The reaction mixture was transferred to 8 kDa molecular weight cutoff dialysis tubing in 3 mL nanopure water, and the solution was dialyzed against H₂O (500 mL×3, solvent exchange every 6 h). The dialyzed solution of JQ-BBP-3 was then concentrated to the desired concentration via centrifugation with a filter tube. Alternatively, JQ-BBP-3 can also be acquired by lyophilization.

### Example 1.14. Synthesis of AZ-BBP

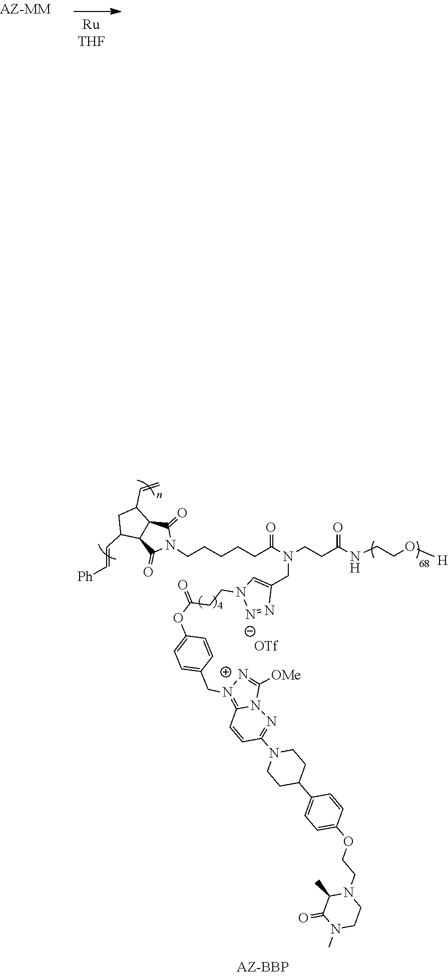

To a vial containing a stir bar, AZ-MM (6.6 mg, 1.56 μmol, 10.0 eq) was added. To another vial, a solution of 3^{rd} generation Grubbs catalyst (Ru, 0.02 M in THF) was freshly prepared. THF (23.3 μL) was then added to the vial containing AZ-MM, followed by the addition of Ru solution (7.8 μL, 0.16 μmol, 1.0 eq) to give the desired DP of 10, while achieving a total AZ-MM concentration of 0.05 M. The yellow reaction mixture was allowed to stir for 3 hours at room temperature. To quench the polymerization, a drop of ethyl vinyl ether was then added. The reaction mixture was transferred to 8 kDa molecular weight cutoff dialysis tubing in 3 mL nanopure water, and the solution was dialyzed against H₂O (500 mL×3, solvent exchange every 6 h). The dialyzed solution of AZ-BBP was then concentrated to the desired concentration via centrifugation with a filter tube. Alternatively, AZ-BBP can also be acquired by lyophilization.

### Example 1.15. Synthesis of Vin-BBP

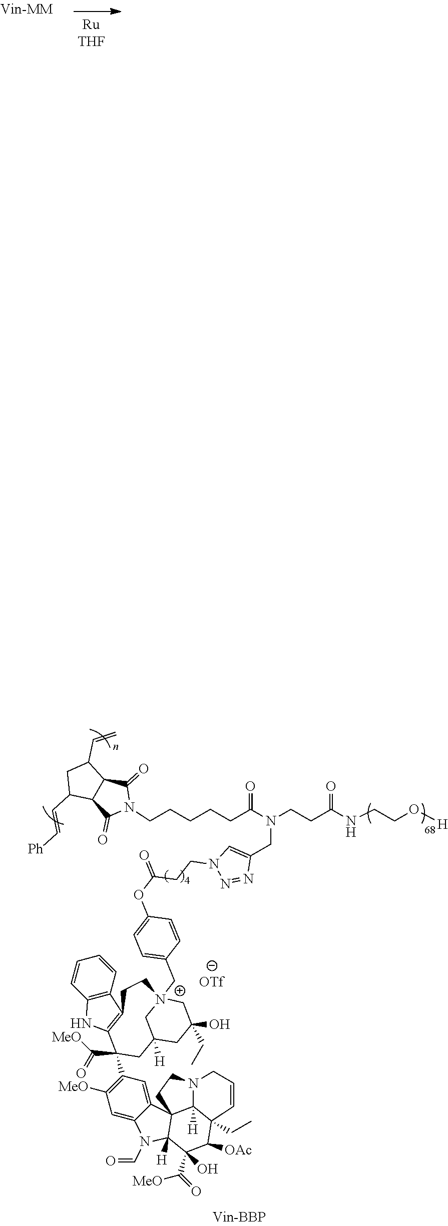

To a vial containing a stir bar, Vin-MM (6.1 mg, 1.4 μmol, 10.0 eq) was added. To another vial, a solution of 3^{rd} generation Grubbs catalyst (Ru, 0.02 M in THF) was freshly prepared. THF (20.4 μL) was then added to the vial containing Vin-MM, followed by the addition of Ru solution (6.8 μL, 0.13 μmol, 1.0 eq) to give the desired DP of 10, while achieving a total Vin-MM concentration of 0.05 M. The yellow reaction mixture was allowed to stir for 3 hours at room temperature. To quench the polymerization, a drop of ethyl vinyl ether was then added. The reaction mixture was transferred to 8 kDa molecular weight cutoff dialysis tubing in 3 mL nanopure water, and the solution was dialyzed against H₂O (500 mL×3, solvent exchange every 6 h). The dialyzed solution of Vin-BBP was then concentrated to the desired concentration via centrifugation with a filter tube. Alternatively, Vin-BBP can also be acquired by lyophilization.

### Example 2.1. Synthesis of CF₃BETi-Proline-N₃ (CF₃BETi-Loaded)

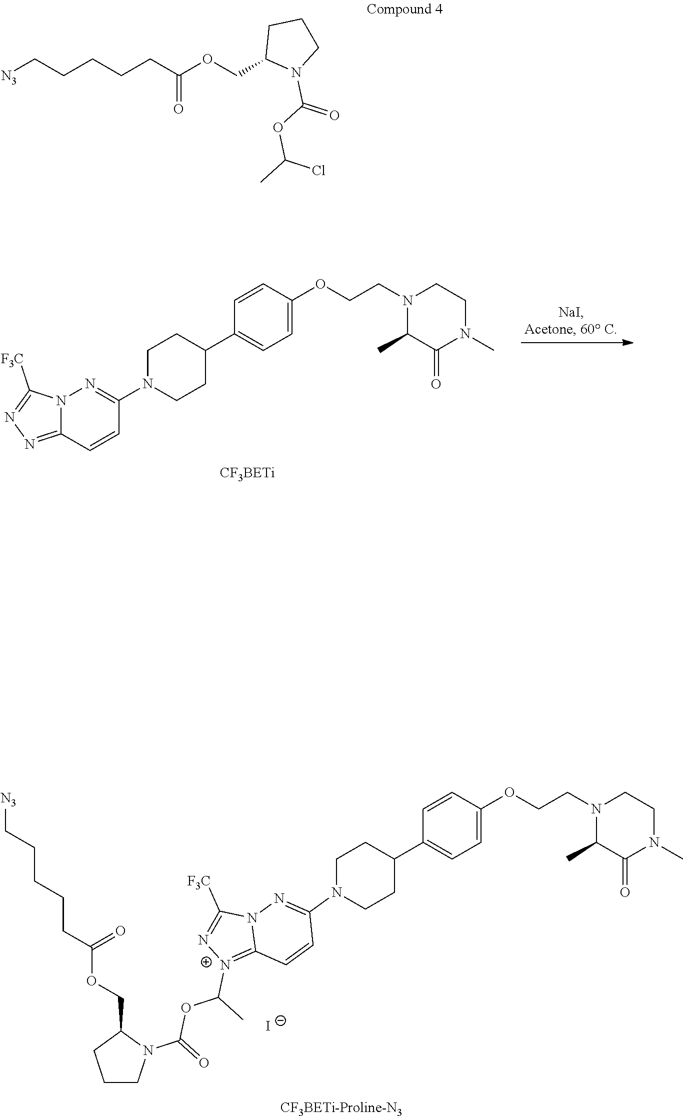 [SMILES: CC(Cl)OC(=O)N1CCC[C@H]1COC(=O)CCCCCN=[N+]=[N-] ; C[C@@H]1C(=O)N(C)CCN1CCOc1ccc(C2CCN(c3ccc4nnc(C(F)(F)F)n4n3)CC2)cc1 ; "CF3BETi" ; CC(OC(=O)N1CCC[C@H]1COC(=O)CCCCCN=[N+]=[N-])[n+]1nc(C(F)(F)F)n2nc(N3CCC(c4ccc(OCCN5CCN(C)C(=O)[C@H]5C)cc4)CC3)ccc21 ; [I-] ; "CF3BETi-Proline-N3"]

To an oven dried N₂ filled vial, Compound 4 (74. mg, 0.21 mmol) and CF₃BETi (110 mg, 0.21 mmol) was added. Acetone (0.80 mL) was then added to the vial and the mixture was stirred until homogenous. NaI (6.3 mg, 0.042 mmol) was then added and the reaction was heated to 60° C. and left to react for 8 hours. Reaction progress was monitored by ¹H NMR and TLC. After completion, additional NaI (62 mg, 0.42 mmol) was added. The reaction was stirred for another hour. The reaction mixture was filtered, diluted with chloroform (3 mL), and filtered through a syringe filter. The crude mixture was then purified by preparatory GPC to yield the product as a brown solid (41 mg, 0.043 mmol, 20% yield). ¹H NMR (600 MHz, CDCl₃, ppm) 6H 9.05-8.97 (overlap, 1H), 8.03-7.97 (overlap, 1H), 7.60 (q), 7.50 (q), 7.45 (q), 7.11 (d, 2H), 6.85 (d, 2H), 4.54-4.47 (overlap), 4.32 (m), 4.15-4.12 (overlap), 4.10-4.04 (overlap), 3.99-3.93 (overlap), 3.73 (overlap, 1H), 3.69-3.62 (overlap), 3.46-3.41 (overlap), 3.38-3.21 (overlap), 3.16-3.11 (overlap), 3.04-3.00 (overlap), 2.93 (s, 3H), 2.88-2.76 (overlap), 2.38-2.33 (overlap, 1H), 2.19-2.09 (overlap), 2.06-1.98 (overlap), 1.98-1.82 (overlap), 1.82-1.72 (overlap), 1.69-1.49 (overlap), 1.44-1.38 (overlap), 1.36-1.21 (overlap). ¹³C NMR (150 MHz, CDCl3, ppm): δC 73.28, 173.21, 170.53, 157.48, 156.65, 156.63, 152.82, 152.51, 152.45, 140.54, 140.42, 140.27, 136.84, 127.81, 127.79, 123.02, 122.90, 122.70, 122.58, 122.49, 122.32, 114.82, 80.94, 80.83, 80.59, 80.25, 70.68, 66.55, 64.26, 64.20, 63.70, 63.19, 60.62, 56.83, 56.73, 56.52, 56.15, 52.89, 51.35, 51.29, 51.24, 48.24, 47.51, 47.12, 47.06, 46.78, 41.37, 34.60, 34.17, 34.09, 34.05, 33.68, 32.99, 28.90, 28.86, 28.68, 28.67, 28.63, 28.56, 27.82, 26.37, 26.35, 26.11, 24.54, 24.48, 24.45, 24.19, 24.08, 23.75, 23.36, 22.97, 18.98, 18.84, 18.68, 18.54, 15.46. Note: NMRs are complex due to rotamers and diastereomers.

### Example 2.2. Synthesis of CF₃BETi-Proline-MM (AZ5153-Loaded)

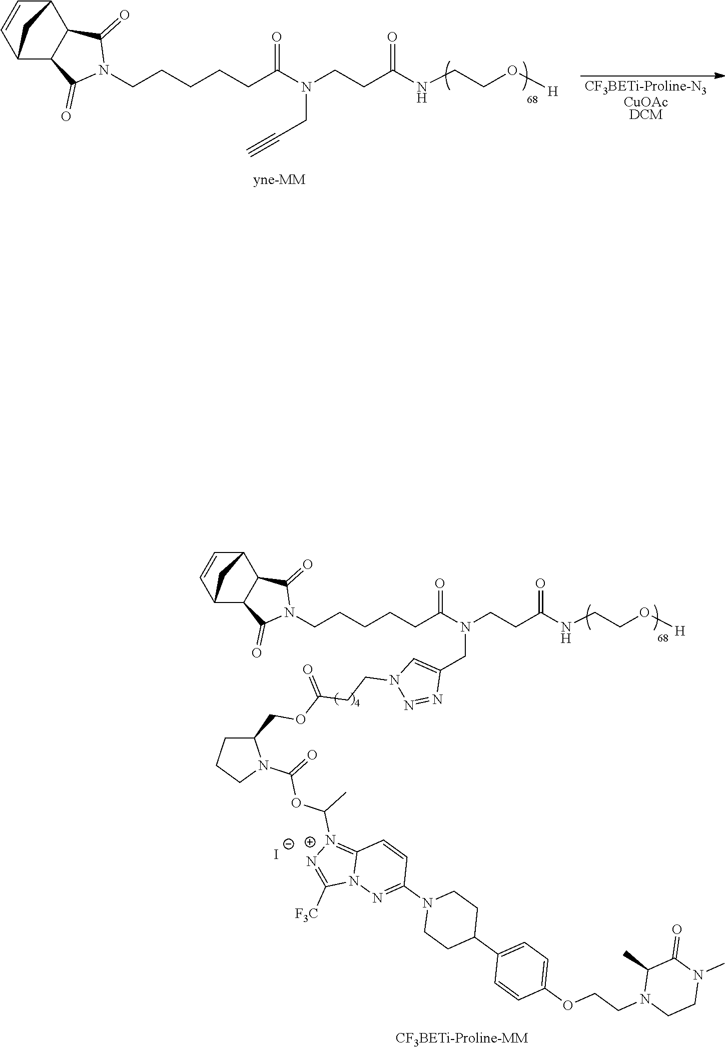

To a vial, yne-MM (129 mg, 0.0381 mmol, 1.0 eq), CF₃BETi-Proline-N₃ (40. mg, 0.044 mmol, 1.15 eq), and DCM (5.0 mL) were added. CuOAc (a pinch) was then added and the reaction mixture was stirred under N₂ atmosphere. The reaction was complete in ˜1 h as determined by LC-MS. The crude mixture was ran through an aluminum oxide plug. The collected solution was concentrated under vacuum, redissolved in CHCl₃, filtered through a 0.45 μm filter (Nalgene), and subjected to recycling preparative HPLC. The fractions containing the product were concentrated under vacuum and dried overnight, affording the product as a solid.

### Example 2.3. Synthesis of CF₃BETi-Proline-BBP

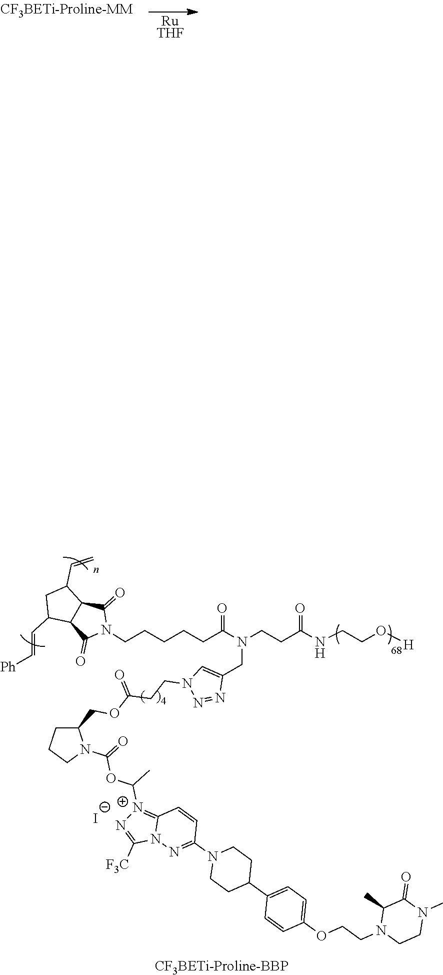

To a vial containing a stir bar, CF₃BETi-Proline-MM (10. mg, 2.3 μmol, 10, eq) was added. To another vial, a solution of 3^{rd} generation Grubbs catalyst (Ru, 0.02 M in THF) was freshly prepared. THF (34.9 μL) was then added to the vial containing CF₃BETi-Proline-MM, followed by the addition of Ru solution (11.5 μL, 0.233 μmol, 1.0 eq) to give the desired DP of 10, while achieving a total CF₃BETi-Proline-MM concentration of 0.05 M. The yellow reaction mixture was allowed to stir for 3 hours at room temperature. To quench the polymerization, a drop of ethyl vinyl ether was then added. The reaction mixture was transferred to 8 kDa molecular weight cutoff dialysis tubing in 3 mL nanopure water, and the solution was dialyzed against H₂O (500 mL×3, solvent exchange every 6 h). The dialyzed solution of CF₃BETi-Proline-BBP was then concentrated to the desired concentration via centrifugation with a filter tube. Alternatively, CF₃BETi-Proline-BBP can also be acquired by lyophilization.

### Example 3.1. Synthesis of CF₃BETi-oBz-N₃ (CF₃BETi-Loaded)

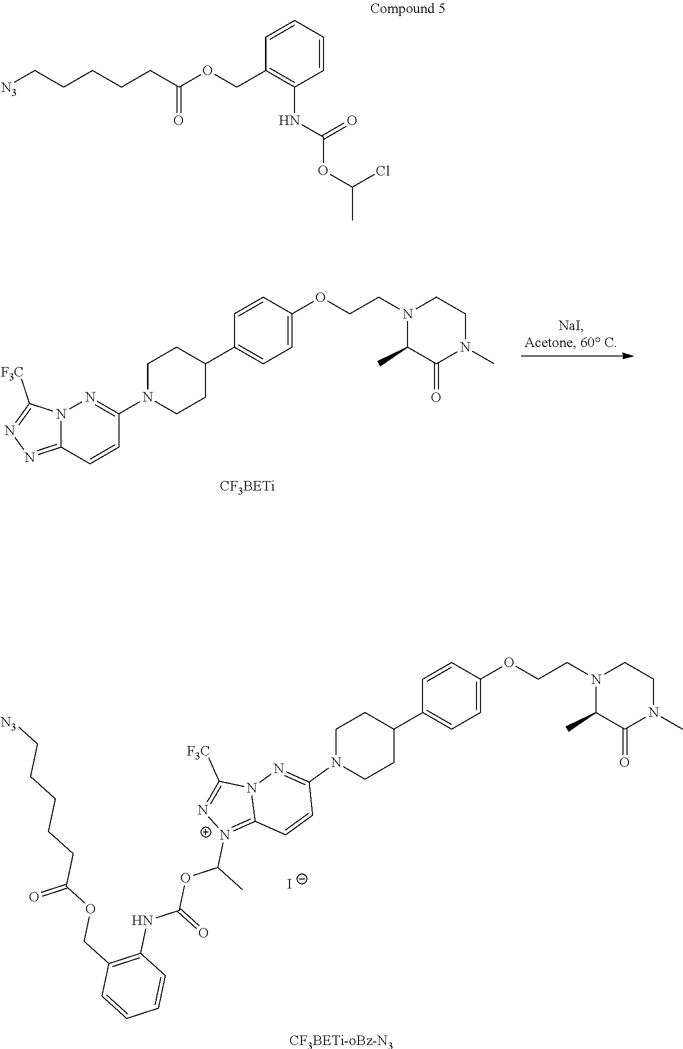 [SMILES: CC(Cl)OC(=O)Nc1ccccc1COC(=O)CCCCCN=[N+]=[N-] ; C[C@@H]1C(=O)N(C)CCN1CCOc1ccc(C2CCN(c3ccc4nnc(C(F)(F)F)n4n3)CC2)cc1 ; "CF3BETi" ; CC(OC(=O)Nc1ccccc1COC(=O)CCCCCN=[N+]=[N-])[n+]1nc(C(F)(F)F)n2nc(N3CCC(c4ccc(OCCN5CCN(C)C(=O)[C@H]5C)cc4)CC3)ccc21 ; [I-] ; "CF3BETi-oBz-N3"]

To an oven dried N₂ filled vial, Compound 5 (50. mg, 0.14 mmol) and CF₃BETi (70. mg, 0.14 mmol) was added. Acetone (0.60 mL) was then added to the vial and the mixture was stirred until homogenous. NaI (4.2 mg, 0.028 mmol) was then added and the reaction was heated to 60° C. and left to react for 8 hours. Reaction progress was monitored by ¹H NMR and TLC. After completion, additional NaI (42 mg, 0.28 mmol) was added. The reaction was stirred for another hour. The reaction mixture was filtered, diluted with chloroform (3 mL), and filtered through a syringe filter. The crude mixture was then purified by preparatory GPC to yield the product as a brown solid (23 mg, 0.023 mmol, 17% yield). Note: NMRs are complex due to rotamers and diastereomers.

### Example 3.2. Synthesis of CF₃BETi-oBz-MM (CF₃BETi-Loaded)

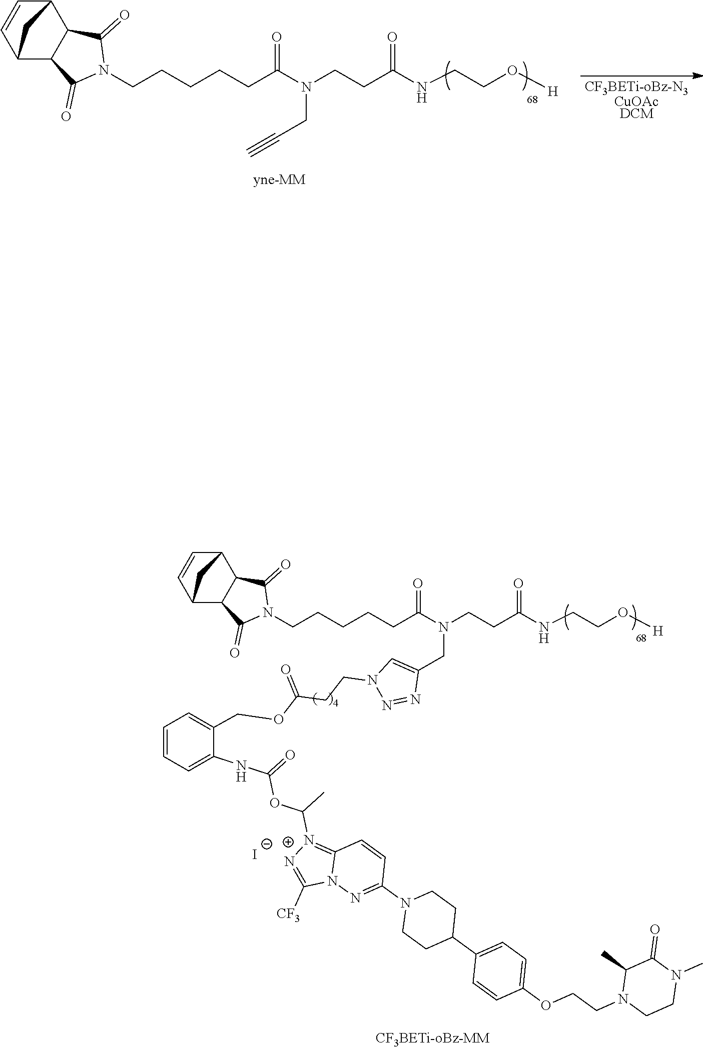

To a vial, yne-MM (57 mg, 0.0168 mmol, 1.0 eq), CF₃BETi-oBz-N₃ (18 mg, 0.019 mmol, 1.15 eq), and DCM (2.5 mL) were added. CuOAc (a pinch) was then added and the reaction mixture was stirred under N₂ atmosphere. The reaction was complete in ˜1 h as determined by LC-MS. The crude mixture was ran through an aluminum oxide plug. The collected solution was concentrated under vacuum, redissolved in CHCl₃, filtered through a 0.45 μm filter (Nalgene), and subjected to recycling preparative HPLC. The fractions containing the product were concentrated under vacuum and dried overnight, affording the product as a solid.

### Example 3.3. Synthesis of CF₃BETi-oBz-BBP

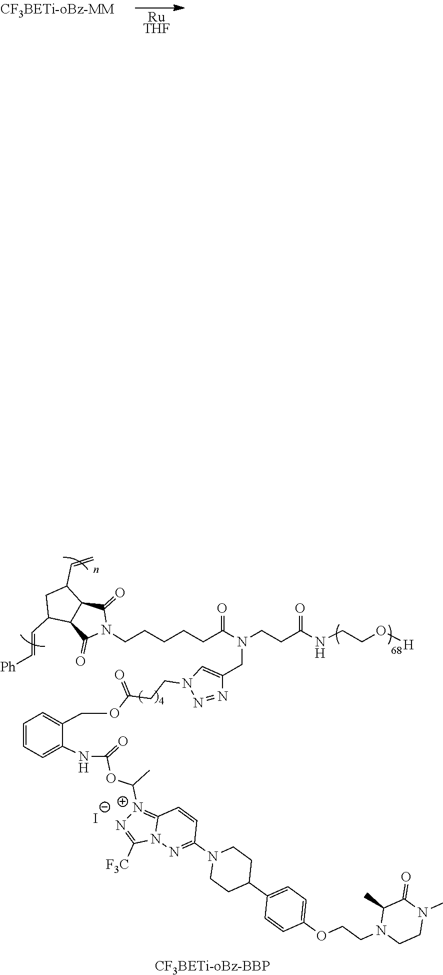

To a vial containing a stir bar, CF₃BETi-oBz-MM (4.4 mg, 1.0 μmol, 10, eq) was added. To another vial, a solution of 3^{rd} generation Grubbs catalyst (Ru, 0.02 M in THF) was freshly prepared. THF (15.3 μL) was then added to the vial containing CF₃BETi-oBz-MM, followed by the addition of Ru solution (5.06 μL, 0.103 μmol, 1.0 eq) to give the desired DP of 10, while achieving a total CF₃BETi-oBz-MM concentration of 0.05 M. The yellow reaction mixture was allowed to stir for 3 hours at room temperature. To quench the polymerization, a drop of ethyl vinyl ether was then added. The reaction mixture was transferred to 8 kDa molecular weight cutoff dialysis tubing in 3 mL nanopure water, and the solution was dialyzed against H₂O (500 mL×3, solvent exchange every 6 h). The dialyzed solution of CF₃BETi-oBz-BBP was then concentrated to the desired concentration via centrifugation with a filter tube. Alternatively, CF₃BETi-oBz-BBP can also be acquired by lyophilization.

### REFERENCES

(1) Rzayev, J.: Molecular Bottlebrushes: New Opportunities in Nanomaterials Fabrication. ACS Macro Letters 2012, 1, 1146-1149.
(2) Sheiko, S. S.; Sumerlin, B. S.; Matyjaszewski, K.: Cylindrical molecular brushes: Synthesis, characterization, and properties. Progress in Polymer Science 2008, 33, 759-785.
(3) Lee, H.-i.; Pietrasik, J.; Sheiko, S. S.; Matyjaszewski, K.: Stimuli-responsive molecular brushes. Progress in Polymer Science 2010, 35, 2444.
(4) Xia, Y.; Olsen, B. D.; Kornfield, J. A.; Grubbs, R. H.: Efficient Synthesis of Narrowly Dispersed Brush Copolymers and Study of Their Assemblies: The Importance of Side Chain Arrangement. Journal of the American Chemical Society 2009, 131, 18525-18532.
(5) Xia, Y.; Kornfield, J. A.; Grubbs, R. H.: Efficient Synthesis of Narrowly Dispersed Brush Polymers via Living Ring-Opening Metathesis Polymerization of Macromonomers. Macromolecules 2009, 42, 3761-3766.
(6) Verduzco, R.; Li, X.; Pesek, S. L.; Stein, G. E.: Structure, function, self-assembly, and applications of bottlebrush copolymers. Chemical Society Reviews 2015, 44, 2405-2420.
(7) Miyake, G. M.; Piunova, V. A.; Weitekamp, R. A.; Grubbs, R. H.: Precisely Tunable Photonic Crystals From Rapidly Self-Assembling Brush Block Copolymer Blends. Angewandte Chemie International Edition 2012, 51, 11246-11248.
(8) Barnes. J. C.; Bruno, P. M.: Nguyen, H. V. T.; Liao, L.; Liu, J.: Hemann, M. T.; Johnson, J. A.: Using an RNAi Signature Assay To Guide the Design of Three-Drug-Conjugated Nanoparticles with Validated Mechanisms, In Vivo Efficacy, and Low Toxicity. Journal of the American Chemical Society 2016, 138, 12494-12501.
(9) Kawamoto, K.; Zhong, M.; Gadelrab, K. R.; Cheng, L.-C.; Ross, C. A.; Alexander-Katz, A.; Johnson, J. A.: Graft-through Synthesis and Assembly of Janus Bottlebrush Polymers from A-Branch-B Diblock Macromonomers. Journal of the American Chemical Society 2016, 138, 11501-11504.
(10) Ren, J. M.; McKenzie, T. G.; Fu, Q.; Wong. E. H. H.; Xu, J.; An. Z.; Shanmugam, S.; Davis, T. P.; Boyer, C.; Qiao, G. G.: Star Polymers. Chemical Reviews 2016, 116, 6743-6836.
(11) Liao, L.; Liu, J.; Dreaden. E. C.; Morton, S. W.; Shopsowitz, K. E.; Hammond, P. T.; Johnson, J. A.: A Convergent Synthetic Platform for Single-Nanoparticle Combination Cancer Therapy: Ratiometric Loading and Controlled Release of Cisplatin, Doxorubicin, and Camptothecin. Journal of the American Chemical Society 2014, 136, 5896-5899.
(12) Sveinbjönsson, B. R.; Weitekamp, R. A.; Miyake, G. M.; Xia, Y.; Atwater. H. A.; Grubbs, R. H.: Rapid self-assembly of brush block copolymers to photonic crystals. Proceedings of the National Academy of Sciences 2012, 109, 14332-14336.
(13) Fox. M. E.; Szoka, F. C.; Fréchet, J. M. J.: Soluble Polymer Carriers for the Treatment of Cancer: The Importance of Molecular Architecture. Accounts of Chemical Research 2009, 42, 1141-1151.
(14) Peer, D.; Karp, J. M.; Hong, S.; Farokhzad. O. C.; Margalit. R.; Langer, R.: Nanocarriers as an emerging platform for cancer therapy. Nat Nano 2007, 2, 751-760.
(15) Maeda, H.; Bharate, G. Y.; Daruwalla, J.: Polymeric drugs for efficient tumor-targeted drug delivery based on EPR-effect. European Journal of Pharmaceutics and Biopharmaceutics 2009, 71, 409419.
(16) Staben, L. R.; Koenig, S. G.; Lehar, S. M.; Vandlen, R.; Zhang. D.; Chuh, J.; Yu, S.-F.; Ng, C.; Guo. J.; Liu. Y.; Fourie-O'Donohue, A.; Go, M.; Linghu, X.; Segraves, N. L.; Wang, T.; Chen, J.; Wei, B.; Phillips, G. D. L.; Xu, K.; Kozak, K. R.; Mariathasan, S.; Flygare, J. A.; Pillow, T. H.: Targeted drug delivery through the traceless release of tertiary and heteroaryl amines from antibody-drug conjugates. Nature Chemistry 2016, 8, 1112-1119.
(17) Burke, P. J.; Hamilton, J. Z.; Pires. T. A.; Setter, J. R.; Hunter. J. H.; Cochran, J. H.; Waight, A. B.; Gordon, K. A.; Toki, B. E.; Emmerton, K. K.; Zeng, W.; Stone, I. J.; Senter, P. D.; Lyon, R. P.; Jeffrey, S. C.: Development of Novel Quaternary Ammonium Linkers for Antibody-Drug Conjugates. Molecular Cancer Therapeutics, 2016, 15, 938-945.
(18) Tian, L.; Yang, Y.; Wysocki, L. M.; Arnold, A. C.; Hu, A.; Ravichandran. B.; Stemson, S. M.; Looger, L. L.; Lavis, L. D.: Selective esterase-ester pair for targeting small molecules with cellular specificity. Proceedings of the National Academy of Sciences, 2012, 109, 4756-4761.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising,” “including.” and “containing,” and all other tenses thereof, are intended to be open and permits the inclusion of additional possibilities (e.g., elements or steps). Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A macromonomer of Formula (I): 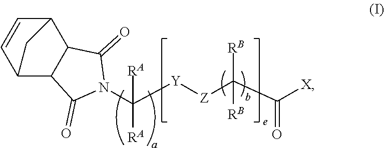
or a salt thereof, wherein:
each instance of R^{A} is independently hydrogen, halogen, or substituted or unsubstituted, C₁₋₆ alkyl;
a is an integer from 1 to 20, inclusive;
each instance of —Y—Z— is independently 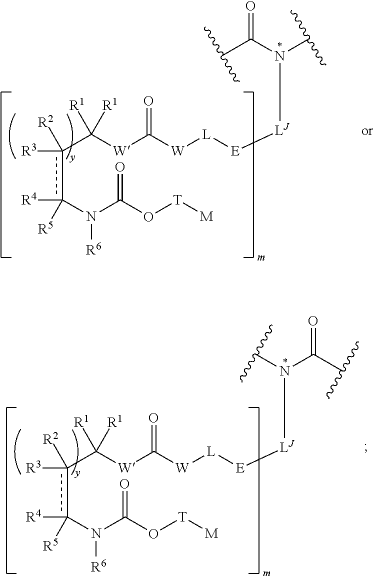
each instance of L^{J} is independently substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, or substituted or unsubstituted, C₂₋₂₀₀ heteroalkynylene, wherein:
optionally one or more carbons in the substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, and substituted or unsubstituted, C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene; and
optionally one or more heteroatoms in the substituted or unsubstituted, C₂₋₂₀₀ heteroalkylene, substituted or unsubstituted, C₂₋₂₀₀ heteroalkenylene, and substituted or unsubstituted, C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene;
each instance of m is independently an integer from 1 to 10, inclusive;
each instance of E is a moiety formed by reacting E^{A} with E^{B};
each instance of E^{A} is a first reaction handle;
each instance of E^{B} is a second reaction handle, wherein the second reaction handle is able to react with the first reaction handle;
each instance of L is independently substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, or substituted or unsubstituted, C₂₋₂₀₀ heteroalkynylene, wherein:
optionally one or more carbons in each instance of the substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkylene, substituted or unsubstituted, C₂₋₂₀₀ heteroalkenylene, and substituted or unsubstituted, C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene; and
optionally one or more heteroatoms in each instance of the substituted or unsubstituted, C₂₋₂₀₀ heteroalkylene, substituted or unsubstituted, C₂₋₂₀₀ heteroalkenylene, and substituted or unsubstituted, C₂₋₂₀₀ heteroalkynylene are independently replaced with substituted or unsubstituted carbocyclylene, substituted or unsubstituted heterocyclylene, substituted or unsubstituted arylene, or substituted or unsubstituted heteroarylene;
each instance of W is independently a single bond, —O—, —S—, or —NR^{E}—;
each instance of R^{E} is independently hydrogen, substituted or unsubstituted, C₁₋₆ alkyl, or a nitrogen protecting group;
each instance of W′ is independently —O— or —S—;
each instance of R¹ is independently hydrogen, halogen, or substituted or unsubstituted, C₁₋₆ alkyl;
each instance of y is independently 0 or 1;
when y is 0, [symbol] is a single bond;
when y is 1, [symbol] is a single or double bond;
when [symbol] is a single bond, R² is hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
each instance of R^{a} is independently hydrogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, an oxygen protecting group when attached to an oxygen atom, a sulfur protecting group when attached to a sulfur atom, or a nitrogen protecting group when attached to a nitrogen atom; or two instances of R^{a} attached to the same nitrogen atom are joined to form substituted or unsubstituted, 3- to 7-membered, monocyclic heterocyclyl, or substituted or unsubstituted, 5- or 6-membered, monocyclic heteroaryl;
when [symbol] is a double bond, R² is absent;
each instance of R³ is independently hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
each instance of R⁴ is independently hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
or, when y is 1 and [symbol] is a single bond, R³ and R⁴ are joined with their intervening atoms to form substituted or unsubstituted carbocyclyl or substituted or unsubstituted heterocyclyl;
or, when y is 1 and [symbol] is a double bond, R³ and R⁴ are cis to each other and are joined with their intervening atoms to form substituted or unsubstituted carbocyclyl, substituted or unsubstituted heterocyclyl, substituted or unsubstituted aryl, or substituted or unsubstituted heteroaryl;
or R⁴ and R⁶ are joined with their intervening atoms to form substituted or unsubstituted heterocyclyl;
when [symbol] is a single bond, R⁵ is hydrogen, halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
when [symbol] is a double bond, R⁵ is absent;
each instance of R⁶ is independently hydrogen, substituted or unsubstituted, C₁₋₆ alkyl, or a nitrogen protecting group;
each instance of T is independently substituted or unsubstituted methylene;
each instance of M is independently an ammonium salt or iminium salt of a pharmaceutical agent, wherein the attachment point is the N′ of the ammonium salt or iminium salt;
each instance of R^{B} is independently hydrogen, halogen, or substituted or unsubstituted, C₁₋₆ alkyl;
each instance of b is independently an integer from 1 to 20, inclusive;
e is an integer from 1 to 10, inclusive;
X is OR^{C} or N(R^{D})₂;
R^{C} is hydrogen, substituted or unsubstituted, C₁₋₁₀₀₀ alkyl, substituted or unsubstituted, C₂₋₁₀₀₀ alkenyl, substituted or unsubstituted, C₂₋₁₀₀₀ alkynyl, substituted or unsubstituted, C₁₋₁₀₀₀ heteroalkyl, substituted or unsubstituted, C₂₋₁₀₀₀ heteroalkenyl, substituted or unsubstituted, C₂₋₁₀₀₀ heteroalkynyl, an oxygen protecting group, or a leaving group; and
each instance of R^{D} is independently hydrogen, substituted or unsubstituted, C₁₋₁₀₀₀ alkyl, substituted or unsubstituted, C₂₋₁₀₀₀ alkenyl, substituted or unsubstituted, C₂₋₁₀₀₀ alkynyl, substituted or unsubstituted, C₁₋₁₀₀₀ heteroalkyl, substituted or unsubstituted, C₂₋₁₀₀₀ heteroalkenyl, substituted or unsubstituted, C₂₋₁₀₀₀ heteroalkynyl, or a nitrogen protecting group.

2-9. (canceled)

10. The macromonomer of claim 1, or a salt thereof, wherein the macromonomer is of the formula: 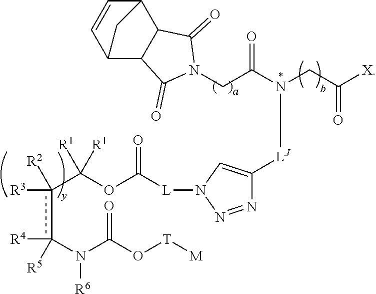
or a salt thereof.

11. The macromonomer of claim 1, or a salt thereof, wherein the macromonomer is of the formula: 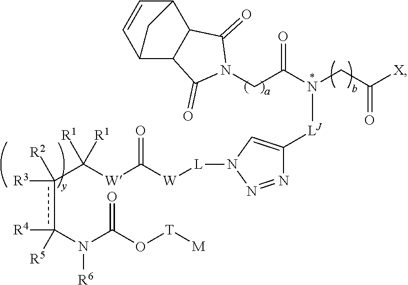
or a salt thereof.

12. The macromonomer of claim 1, or a salt thereof, wherein the macromonomer is of the formula: 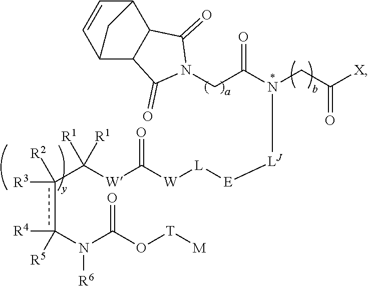
or a salt thereof.

13. The macromonomer of claim 1, or a salt thereof, wherein the macromonomer is of the formula: 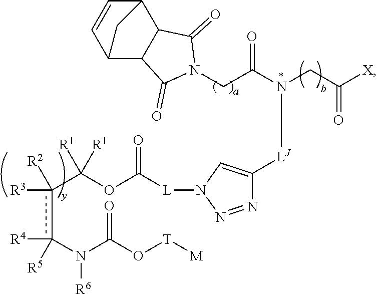
or a salt thereof.

14. The macromonomer of claim 1, or a salt thereof, wherein the macromonomer is of the formula: 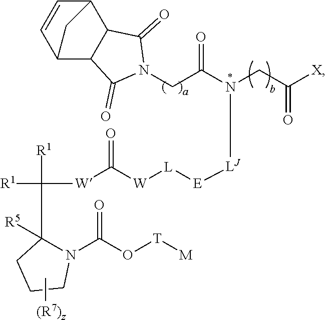
or a salt thereof, wherein:
each instance of R⁷ is independently halogen, substituted or unsubstituted, C₁₋₆ alkyl, substituted or unsubstituted, C₂₋₆ alkenyl, substituted or unsubstituted, C₂₋₆alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂; and
each instance of z is independently 0, 1, 2, 3, 4, 5, or 6.

15. The macromonomer of claim 1, or a salt thereof, wherein the macromonomer is of the formula: 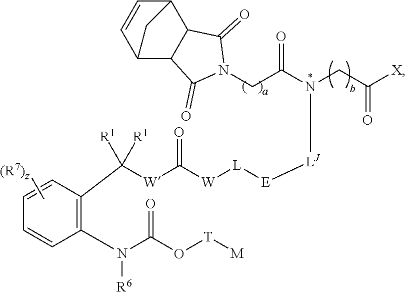
or a salt thereof, wherein:
each instance of R⁷ is independently halogen, substituted or unsubstituted, C₁₋₆ alkyl, substituted or unsubstituted, C₂₋₆ alkenyl, substituted or unsubstituted, C₂₋₆ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂; and
each instance of z is independently 0, 1, 2, 3, or 4.

16. The macromonomer or salt of claim 1, wherein each instance of R^{A} an R^{B} is hydrogen.

17-21. (canceled)

22. The macromonomer or salt of claim 1, wherein at least one instance of L^{J} is unsubstituted C₁₋₄ alkylene.

23. (canceled)

24. The macromonomer or salt of claim 1, wherein at least one instance of E is a moiety formed by reacting two click-chemistry handles.

25. (canceled)

26. The macromonomer or salt of claim 1, wherein at least one instance of E is

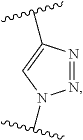 [SMILES: *c1cn(*)nn1]

27-39. (canceled)

40. The macromonomer or salt of claim 1, wherein each instance of L is independently unsubstituted C₂₋₂₀ alkylene or —CH₂CH₂—(O—CH₂CH₂)₁₋₆—.

41. The macromonomer or salt of claim 1, wherein at least one instance of W is a single bond.

42. The macromonomer or salt of claim 1, wherein at least one instance of W′ is —O—.

43-44. (canceled)

45. The macromonomer or salt of claim 1, wherein:
y is 0; and
R⁴ and R⁶ are joined with their intervening atoms to form substituted or unsubstituted heterocyclyl.

46. (canceled)

47. The macromonomer or salt of claim 1, wherein:
y is 1; [symbol]
is a double bond; and
R³ and R⁴ are cis to each other and are joined with their intervening atoms to form substituted or unsubstituted aryl or substituted or unsubstituted heteroaryl.

48-54. (canceled)

55. The macromonomer or salt of claim 1, wherein at least one instance of T is —CH₂—.

56-72. (canceled)

73. The macromonomer or salt of claim 1, wherein X is 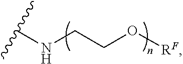
wherein n is an integer from 40 to 100, inclusive; and R^{F} is hydrogen or unsubstituted, C₁₋₆ alkyl.

74-78. (canceled)

79. A method of preparing a macromonomer of claim 1, or a salt thereof, comprising coupling a compound of the formula: 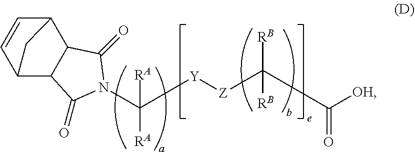
or a salt thereof, with a compound of the formula: HOR^{C} or HN(R^{D})₂, or a salt thereof.

80-94. (canceled)

95. A kit comprising:
a macromonomer of claim 1, or a salt thereof; and
instructions for using the macromonomer, or a salt thereof.

96-112. (canceled)
